(19) United States
(12) Patent Application Publication
(10) Pub. No.: US 2018/0059115 A1
(43) Pub. Date: Mar. 1, 2018

(54) METHODS FOR MONITORING POLYMORPHONUCLEAR MYELOID DERIVED SUPPRESSOR CELLS, AND COMPOSITIONS AND METHODS OF TREATMENT OF CANCER

(71) Applicant: The Wistar Institute of Anatomy and Biology, Philadelphia, PA (US)

(72) Inventors: Dmitry I. Gabrilovich, Villanova, PA (US); Thomas C. Condamine, Philadelphia, PA (US)

(21) Appl. No.: 15/668,867

(22) Filed: Aug. 4, 2017

Related U.S. Application Data
(60) Provisional application No. 62/371,493, filed on Aug. 5, 2016.

(51) Int. Cl. G01N 33/574; C07K 16/28; C12Q 1/68

(52) U.S. Cl. CPC G01N 33/57492; C07K 16/28; C12Q 1/6886; C12Q 2600/158; G01N 2333/705; G01N 2333/70596; C07K 2317/76

(57) ABSTRACT

A method of obtaining a population of cells enriched in human polymorphonuclear myeloid derived suppressor cells (PMN-MDSCs) comprises isolating from a cell suspension those cells which express LOX-1 to provide a population of cells enriched with PMN-MDSCs. A method of monitoring the population of LOX-1+ cells in a cell-containing biological sample is useful for determining the efficacy of treatment or the metastasis or increasing progression of cancer. Other cell isolation and diagnostic methods are also described. A composition for use in diagnosing and treating cancer related to PMN-MDSC is provided that contains antagonists and/or inhibitors of genes related to the ER stress response.

### CROSS-REFERENCE TO RELATED APPLICATIONS

This application claims the benefit of the priority of U.S. Provisional Patent Application No. 62/371,493, filed Aug. 5, 2016, which application is incorporated herein by reference.

### STATEMENT REGARDING FEDERALLY SPONSORED RESEARCH OR DEVELOPMENT

This invention was made with government support under Grant Nos. CA084488, CA100062 and CA010815, awarded by the National Institutes of Health. The government has certain rights in this invention.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “WST165US ST25.txt”, created Aug. 3, 2017, and having 4 KB.

### BACKGROUND OF THE INVENTION

Myeloid-derived suppressor cells (MDSC) represent a heterogeneous population of immature myeloid cells. These cells accumulate to a great extent in cancer patients and play a major role in regulating immune responses in cance^{r42}. MDSC suppress T cells activation and proliferation as well as function of natural killer (NK) cells^{14,15}. Ample evidence links these cells with tumor progression and outcome of the disease in cancer patients^{34,15}. The accumulation of relatively immature and pathologically activated myeloid-derived suppressor cells (MDSC) with potent immunosuppressive activity is common in tumors. MDSC have the ability to support tumor progression by promoting tumor cell survival, angiogenesis, invasion of healthy tissue by tumor cells, and metastases⁴. There is now ample evidence of the association of accumulation of immune suppressive MDSC with negative clinical outcomes in various cancers³². MDSC have been implicated in resistance to anticancer therapies with kinase inhibitor¹¹, chemotherapy^{9,47,23,8}, and immune therapy^{33,44,50,12,20}.

MDSC have been divided in two large sub-populations⁵², monocytic myeloid-derived suppressor cells (M-MDSC) and polymorphonuclear myeloid-derived suppressor cells (PMN-MDSC). About 20-30% of MDSC consists of monocytic cells, i.e., M-MDSC, and are generally associated with high activity of Arginase-1 and iNOS¹⁰. Two different phenotypes (CD11b⁺CD14⁻CD15⁻ and CD33⁺ or CD11b⁺CD14⁺CD33⁺ and HLA-DR^{lo}) are used to characterize these M-MDSC cells depending on the type of cancer.

The second population, i.e., PMN-MDSC, are comprised of granulocytic cells and are usually associated with high level of ROS production³⁶. PMN-MDSC represent the major population of MDSC (about 60-80%) and represent the most abundant population of MDSC in most types of cancer. PMN-MDSC are phenotypically and morphologically similar to neutrophils (PMN)³⁶ and share the CD11b+CD14-CD15+/CD66b+ phenotype. The may also be characterized as CD33⁺. PMN-MDSC are important regulators of immune responses in cancer and have been directly implicated in promotion of tumor progression. However, the heterogeneity of these cells and lack of distinct markers hampers the progress in understanding of the biology and clinical significance of these cells. One of the major obstacles in the identification of PMN-MDSC is that they share the same phenotype with normal polymorphonuclear cells (PMN).

Distinction between PMN-MDSC and PMN in tumor tissues is not possible. Currently, these cells can be separated only in peripheral blood (PB) and only by density gradient. Since gradient centrifugation may enrich not only for true PMN-MDSC, but also for activated PMN without suppressive activity, the heterogeneity of PMN-MDSC population raised the questions of whether PMN-MDSC and PMN are truly cells with distinct features. It is not clear what defines the specific functional state of human PMN-MDSC PMN in the same patient. More importantly, the mechanisms responsible for acquisition of pathological activity by human neutrophils in cancer remained unclear.

Current methods for separating populations of PMN-MDSC from populations of PMN in biological samples are complicated, time-consuming and inaccurate, requiring multiple gradient separation as well as multi-color flow cytometry analysis. Normal PMN have high density and pass through the gradient, whereas PMN-MDSC have lower density become trapped on the gradient together with mononuclear cells. This process of distinguishing between the two sets of PMN has two major shortcomings. The density of the cells depends on many parameters, such as conditions for collection, time of storage, etc., which affect the proportion of the cells obtained on the gradient regardless of their PMN-MDSC true state. These conditions thus introduce errors into the analysis. Additionally, these processes are inconvenient and difficult to standardize. Thus, there are no useful methods currently exist that allow for discrimination of these two populations in blood and tissues.

### SUMMARY OF THE INVENTION

In one aspect, a method for monitoring the population of polymorphonuclear myeloid derived suppressor cells (PMN-MDSCs) in a mammalian subject involves contacting a biological sample from the subject containing polymorphonuclear neutrophils (PMNs) and PMN-MDSC with a ligand that specifically binds or forms a complex with LOX-1 on the cell surface. Detecting and distinguishing the complexes of ligand-bound LOX-1-cells from other cells not bound to the ligand in the sample enables the tracking of the number or changes in the number of PMN-MDSCs substantially free of PMN.

In another aspect, a method of differentiating polymorphonuclear myeloid derived, suppressor cells (PMN-MDSCs) from polymorphonuclear neutrophils (PMNs) in a biological sample containing both types of cells involves contacting the sample with a ligand that specifically binds or forms a complex with LOX-1on the cell surface. The LOX-1-bound cells can be detected, identified, or measured apart from other cells not bound to the ligand in the sample. The LOX-1-bound cells are PMN-MDSCs substantially free of PMN.

In another aspect, a method of obtaining a population of PMN-MDSC from a biological sample containing other cell types comprises isolating from a cell suspension those cells which express LOX-1 to provide a population of cells enriched with PMN-MDSCs.

In another aspect, a method for differential diagnosis of cancer comprises contacting a biological sample of a subject with reagents capable of complexing or binding with LOX-1 on the surface of a cell; and detecting or measuring any cells that complex with the reagent. Cells that form a complex with the LOX-1 reagent indicate the presence of cancer cells in the sample.

In another aspect, a substantially pure population of PMN-DMSCs is produced by isolating LOX-1⁺ cells from a biological sample by contacting the sample with a reagent that forms a complex or binds to LOX-1.

In a further aspect, a pharmaceutical composition is provided that reduces or inhibits ER stress in mammalian neutrophils or reduces or inhibits LOX-1 expression on LOX-1+ neutrophil populations, LOX-1+ PMN and/or PMN-MDSC in a pharmaceutically acceptable carrier or excipient. In certain embodiments, the composition comprises an antagonist or inhibitor of the expression, activity or activation of one or more of sXBP1, DDIT3 (CHOP), ATF4, ATF3, SEC61A ARGI or NOS-2. In other embodiments, the composition comprises an antagonist or inhibitor of LOX-1 or an antagonist or inhibitor of the expression, activity, or activation of one or more of MYCN, CSF3, IL3, TGFβ1, TNF, LDL, RAF1, APP, IL6 PDGFBB, EPO, CD40LG, NFkB, IL13, AGT, IL1β, ERBB2, MAP2K1, VEGFα, CSF1, FLI1, or IFNγ.

In another aspect, a method for reducing or inhibiting LOX-1+ PMN-MDSC accumulation in a cancer patient comprises administering a composition as described herein.

A method of diagnosing a mammalian subject with a cancer comprises detecting and distinguishing the complexes of antibody-bound LOX-1-cells from other cells not bound to the antibody in the sample, and determining the size of a tumor in the subject by correlation with the number of LOX-1+ PMN or PMN-MDSC detected.

In another embodiment, a method of diagnosing and treating a cancer comprises diagnosing the subject with cancer when the presence of LOX-1+ is detected at a level that indicates PMN-MDSC are present; and administering an effective amount of a composition that reduces or inhibits ER stress response in mammalian neutrophils or reduces or inhibits LOX-1 expression on neutrophil populations.

Other aspects and advantages of these compositions and methods are described further in the following detailed description of the preferred embodiments thereof.

### BRIEF DESCRIPTION OF THE DRAWINGS

FIG. 1 is a graph showing the proportion of LOX-1 positive cells among CD11b⁺CD14⁻CD33⁺CD15⁺ polymorphonuclear cells (PMN) and PMN-MDSC in 23 cancer patients (PMN, ▪; and PMN-MDSC, ▴) and PMN in 9 healthy donors (HD, ). Peripheral blood was subjected to gradient centrifugation using Ficcol and Percoll gradients. PMN-MDSC are evaluated in mononuclear fraction and PMN in granulocytic fraction as described in the text. The proportion of LOX-1 positive cells was evaluated by flow cytometry. **** - p<0.0001 between patients PMN-MDSC and PMN.

FIG. 2A is a graph showing the percentage of LOX-1 positive cells among CD11b⁺CD14⁻CD33⁺CD15⁺ PMN (▪) and PMN-MDSC (▴) in 5 head and neck cancer patients and PMN () in 9 healthy donors (HD). The data is shown as in FIG. 1 but separated based on the cancer types. ** - p<0.01 between patients PMN-MDSC and PMN.

FIG. 2B is a graph showing the percentage of LOX-1 positive cells among CD11b⁺CD14⁻CD33⁺CD15⁺ PNM (▪) and PMN-MDSC (▴) in 9 lung cancer patients and PMN () in 9 healthy donors (HD). The data is shown as in FIG. 1 but separated based on the cancer types. ** - p<0.01 between patients PMN-MDSC and PMN.

FIG. 2C is a graph showing the percentage of LOX-1 positive cells among CD11b⁺CD14⁻CD33⁺CD15⁺ PNM (▪) and PMN-MDSC (▴) in 4 colon cancer patients, and PMN () in 9 healthy donors. The data is shown as in FIG. 1 but separated based on the cancer types. * - p<0.05 between patients PMN-MDSC and PMN.

FIG. 2D is a graph showing the percentage of LOX-1 positive cells among CD11b⁺CD14⁻CD33⁺CD15⁺ PNM (▪) and PMN-MDSC (▴) in 5 breast cancer patients and PMN () in 9 healthy donors. The data is shown as in FIG. 1 but separated based on the cancer types. * - p<0.05 between patients PMN-MDSC and PMN.

FIG. 3 is a graph showing the link between the proportion of LOX-1⁺ cells among PMN-MDSC in 6 early stage (I or II, ▴) cancer patients and 7 late stage (III or IV, ♦) cancer patients. The data is reported as in FIG. 1 but separated based on the stage of cancers. * - p<0.05 between patients with early and late stages of the diseases.

FIG. 4A is a graph showing the proportion of LOX-1⁺CD11b⁺, CD33⁺, CD14⁻, CD15⁺ cells (neutrophils) among all leukocytes in unseparated whole blood. Samples of whole blood were collected from 11 healthy donors () and 12 cancer patients (▪, lung cancer and head and neck cancer. Red cells were lysed and the rest evaluated directly by flow cytometry.

FIG. 4B is a graph showing the proportion of LOX-1⁺CD11b⁺, CD33⁺, CD14⁻, CD15⁺ cells (neutrophils) among all leukocytes in unseparated whole blood. Samples of whole blood were collected from 11 healthy donors (), 5 lung cancer patients (▪), and 5 head and neck cancer patients (H&N, ▴). Red cells were lysed and the rest evaluated directly by flow cytometry. * - p<0.05; *** - p<0.001.

FIG. 4C is a graph showing the proportion of LOX-1⁺CD11b⁺, CD33⁺, CD14⁻, CD15⁺ cells among all neutrophils in unseparated whole blood. Samples of whole blood were collected from 11 healthy donors (▪) and 12 cancer patients (). Red cells were lysed and the rest evaluated directly by flow cytometry. ** - p<0.01.

FIG. 4D is a graph showing the proportion of LOX-1⁺CD11b⁺, CD33⁺, CD14⁻, CD15⁺ cells among all neutrophils in unseparated whole blood. Samples of whole blood were collected from 11 healthy donors (▪), lung cancer (▪) and head and neck cancer (▴) patients. Red cells were lysed and the rest evaluated directly by flow cytometry. * - p<0.05; *** - p<0.001.

FIG. 5A is a bar graph showing that LOX-1+ PMN from cancer patient No. 1 suppresses T cell function. Samples of whole blood were collected from patient with HNC. Red cells were lysed, and PMN were highly enriched by negative selection using Miltenyi bead kit (MACSxpress Neutrophil isolation kit). Cells were then labeled with PE-conjugated LOX-1 antibody followed by anti-PE beads. LOX-1⁺ and LOX-1⁻ PMN were added to mixed allogeneic reaction at indicated ratios and T-cell proliferation was (for LOX-1⁻ PMN, black bar; for LOX-1+ PMN, white bar) measured 5 days later by ³H-thymidin uptake. Each experiment was performed in triplicate. Dashed line—the level of T cell proliferation in the absence of PMN. * - p<0.05 difference from control and from between the group in FIG. 5B and FIG. 5C.

FIG. 5B shows a bar graph for Patient #2 in the experiment described in FIG. 5A using the same symbols.

FIG. 5C shows a bar graph for Patient #3 in the experiment described in FIG. 5A using the same symbols.

FIG. 6A is a graph showing ROS in PMN from head and neck cancer patient No. 15-09. Samples of whole blood were collected. Red cells were lysed and PMN were labeled with CD15, LOX-1 antibodies and DCFDA (to measure ROS). FIG. 6A shows the gating strategy of CD15⁺LOX-1⁺ or LOX-1⁻ cells. Mean fluorescence intensity is shown under the graph.

FIG. 6B is a histogram from patient No. 15-09 showing the intensity of DCFDA fluorescence reflecting the amount of ROS. Mean fluorescence intensity is shown under the histogram.

FIG. 6C is a histogram showing ROS in PMN from head and neck cancer patient No. 15-14. PMN were obtained and labeled as in FIG. 6A. FIG. 6C shows the gating strategy of CD15⁺LOX-1⁺ or LOX-1⁻ cells. Mean fluorescence intensity is shown under the histogram.

FIG. 6D is a histogram from patient No. 15-14 showing the intensity of DCFDA fluorescence reflecting the amount of ROS. Mean fluorescence intensity is shown under the histogram.

FIG. 7A shows the correlation between the presence of PMN-MDSC and soluble LOX-1 in sera of 16 lung cancer patients. Concentration of sLOX-1 was measured in sera using ELISA. Proportion of PMN-MDSC was measured as described in FIGS. 2A-2D. R=correlation coefficient Pearson. N=number of pairs analyzed.

FIG. 7B shows the correlation between the presence of PMN-MDSC and soluble LOX-1 in sera of 6 colon cancer patients. Concentration of soluble LOX-1 (sLOX-1) was measured in sera using ELISA. Proportion of PMN-MDSC was measured as described in FIGS. 2A-2D. R=correlation coefficient Pearson. N=number of pairs analyzed.

FIG. 8A is a bar graph showing the results of a suppression assay of PMN and PMN-MDSC isolated from the same patient with HNC. Allogeneic mixed leukocyte reaction was performed as described in Example 7. Cell proliferation was evaluated in triplicates using 3H-thymidine uptake. Mean and SD are shown. * - p<0.05 from control—T cell proliferation without the presence of PMN or PMN-MDSC. Three patients with the same results were evaluated.

FIG. 8B is a bar graph showing suppression assay of PMN and PMN-MDSC isolated from the same patient with NSCLC. T cell proliferation in response to CD3/CD28 was performed as described in Example 7. Cell proliferation was evaluated in triplicates using 3Hthymidine uptake. Mean and SD are shown. * - p<0.05, **-p<0.01; ***- p<0.001 from control—T cell proliferation without the presence of PMN or PMN-MDSC.

FIG. 8C is a hierarchical clustering of PMN-MDSCs from HNC cancer patients indicating a gene expression signature specific to PMN-MDSCs and similarities of PMN from cancer patients and PMN from healthy donors.

FIG. 8D is a hierarchical clustering of PMN-MDSCs from NSCLC cancer patients indicating a gene expression signature specific to PMN-MDSCs and similarities of PMN from cancer patients and PMN from healthy donors.

FIG. 9A shows LOX-1 as a marker of PMN-MDSC in a heatmap of relative expression of candidate surface markers specific to the PMN-MDSCs and forming part of the PMN-MDSC gene signature.

FIG. 9B is a graph showing the proportion of LOX-1 positive PMN and PMN-MDSC in peripheral blood of 15 cancer patients. Cells were isolated using density gradient as described in Example 7 and the proportion of LOX-1+ cells was calculated among CD15+ cells.

FIG. 9C is a graph showing the cumulative results of staining with CD41a and CD42b antibody of 7 patients with NSCLC. Mean and SD are shown. These data show the LOX-1 expression in PMN-MDSC is not associated with platelets adhesion on PMN-MDSC.

FIGS. 9D, 9E and 9F are three graphs showing the proportion of LOX-1+ cells among PMN in unseparated peripheral blood (PB) from 16 healthy donors (HD) and 20 patients with non-small cell lung cancer (NSCLC) (FIG. 9D), 21 patients with head and neck cancer (HNC) (FIG. 9E), and 19 patients with colon cancer (CC) (FIG. 9F). ** - p<0.01; **** - p<0.0001.

FIG. 9G is a graph showing the proportion of LOX-1+ cells among PMN in unseparated PB from 16 healthy donors (HD), 6 patients with eosinophilic colitis (EE), 3 patients with UC, and 3 patients with CD.

FIG. 10A is a transcriptome graph showing that LOX-1 expression defines bona-fide PMN-MDSC. Hierarchical clustering of samples based on expression levels of genes differentially expressed between LOX-1+ and LOX-1− PMN. These genes form part of the PMN-MDSC gene signature.

FIG. 10B is a list and relative expression values of the most changed known genes overlapped between LOX-1+ and PMN-MDSC cells.

FIG. 10C is a bar graph showing the suppressive activity of LOX-1+ and LOX-1− PMN isolated from peripheral blood of patient with HNC in allogeneic MLR. Cell proliferation was evaluated in triplicates using 3H-thymidine uptake. Mean and SE are shown. * - p<0.05 from control; ** - p<0.01; from values of T cell proliferation without the presence of PMN. Experiments with similar results were performed with samples from 7 patients with HNC and NSCLC.

FIG. 10D is a graph showing that ROS production in LOX-1+ and LOX-1− PMN from 7 patients with HNC and NSCLC. ROS production was measured by staining with DCFDA.

FIGS. 10E and 10F are graphs showing expression of ARG1 (FIG. 10F) and NOS2 (FIG. 10G) in LOX-1+ and LOX-1− PMN from 6 patients with HNC and MM measured by qPCR. *- p<0.05.

FIGS. 10G, 10H and 10I show the effect of 1 μM of N-acetyl L-cysteine (NAC) (FIG. 10H), 1000 U/ml of catalase (FIG. 10I) and 20 μM Nor-NOHA (FIG. 10J) on immune suppressive activity of LOX-1+ PMN-MDSC. Allogeneic MLR was used in all experiments. Cell proliferation was measured in triplicates by 3H-thymidine incorporation. 1:2 PMN:T cell ratio was used in all experiments. Three experiments with similar results were performed. ** - p<0.01***-p<0.001 from values without addition of PMN. ##-p<0.01; ###-p<0.001 from values in LOX-1− PMN. ROS and arginase control suppressive activity of LOX-1+ PMN.

FIGS. 11A and 11B show the mechanism regulating LOX-1 expression on PMN-MDSC, specifically the percentage of LOX-1+ PMN (FIG. 11A) and expression of LOX-1 (FIG. 11B) in PMN isolated from 4 healthy donors and treated with indicated cytokines. Range of concentrations based on reported data were tested and only one for each cytokine is shown. Conditioned medium from PCI30 tumor cells (TCM) was used at 20% v/v concentration. Mean and SD are shown.

FIGS. 11C to 11G show the expression of genes involved in ER stress response in PMN (sXBP-1, FIG. 11C; SEC61A, FIG. 11D; ATF4, FIG. 11E; ATF3, FIG. 11F; and CHOP, FIG. 11G) from 8 patients with HNC and NSCLC. * - p<0.05; ** - p<0.01; ***- p<0.001; **** - p<0.0001 between LOX-1- and LOX-1+ PMN.

FIG. 11H shows the percentage of LOX-1+ PMN in PMN isolated from 4 healthy donors and treated with 1 μM THG and 1 mM DTT. Mean and SD are shown. * - p<0.05 ** - p<0.01 from cells cultured in medium alone.

FIG. 11I shows expression of LOX-1 in PMN isolated from 4 healthy donors and treated with 1 μM THG and 1 mM DTT. Mean and SD are shown. * - p<0.05 ** - p<0.01 from cells cultured in medium alone.

FIG. 11J is a graph showing that ER stress inducer THG converted PMN to PMN-MDSC. PMN isolated from healthy donors were treated overnight with 1 μM THG, extensively washed and then used in CD3/CD28 induced T-cell proliferation. T cell proliferation was measured in triplicates by 3H-thymidine uptake. Three experiments with similar results were performed. *** - p<0.001 between treated and untreated PMN.

FIGS. 11K and 11L are bar graphs showing sXBP1 inhibitor B-I09 abrogated THG inducible up-regulation of LOX-1 and T cells suppression in PMN from healthy donors. PMN were incubated together with 20 μM B-I09 and THG overnight followed by evaluation of LOX-1 expression (FIG. 11K) or suppression activity (FIG. 11L). PMN from three healthy donors were used in these experiments. * - p<0.05, ** - p<0.01, *** - p<0.001 between treated and untreated PMN. ER stress induces LOX-1 expression in PMN and converts there cells to suppressive cells.

FIGS. 12A and 12B are two graphs showing LOX-1+ PMN-MDSC in tumor tissues, specifically the correlation of soluble LOX1 in plasma of NSCLC (FIG. 12A) and colon cancer (FIG. 12B) with the presence of PMN-MDSC in PBMC fraction of PB.

FIG. 12C is a graph showing the presence of LOX-1+ PMN in PB and tumor tissues of 10 patients with CC and NSCLC.

FIG. 12D is a graph showing the presence of LOX-1+ PMN in PB and BM of 7 patients with multiple myeloma (MM).

FIG. 12E is a graph showing the suppressive activity of LOX-1+ PMN in BM of patient with MM tested in allogeneic MLR. * - p<0.05, ** - p<0.01 from values without the presence of PMN. Two patients were tested with the same results.

FIG. 12F is a graph showing LOX-1+ PMN in tissues from 4 normal skin samples, 4 samples of tumor-free lymph nodes, 4 samples of normal colon, as well as tumor tissues from 8 patients with melanoma, 8 patients with HNC patients, 8 patients with NSCLC, and 9 patients with CC. Mean and SD are shown. * - p<0.05, ** - p<0.01, *** - p<0.001 between tumor samples and samples from control tissues.

FIG. 13A is a graph showing the Kaplan-Meier survival curves for HNC squamous cell carcinoma patients survival stratified by median OLR1 expression indicates decreased survival for subjects with high OLR1 expression. p=cox-regression p value, HR=hazard ratio

FIG. 13B is a graph showing the proportion of LOX-1+ PMN in PB of 12 patients with stage I-II; 7 patients with stage III-IV of NSCLC, and 16 healthy donors.

FIG. 13C is a graph showing the proportion of LOX-1+ PMN-MDSC in PB of NSCLC patients segregated based on tumor size. * p<0.05, **** - p<0.001.

FIG. 13D is a graph showing the amount of LOX-1+CD15+ PMN-MDSC in tumor tissues from NSCLC patients segregated based on tumor size.

### DETAILED DESCRIPTION OF THE INVENTION

As disclosed herein, methods and compositions are described which are useful in the isolation of certain cells indicative of cancer in a mammalian subject. Cell preparations that are substantially purified PMN-DMSCs are prepared by methods involving the use of reagents that complex with or bind the LOX-1 biomarker on the surface of cells, thereby discriminating between PMN cells and PMN-DMSCs. The methods described herein are also useful for the diagnosis and/or monitoring of cancer and tumor cells, i.e., both malignant and benign tumors, so long as the cells to be treated carry the LOX-1 cell surface antigen. Further, the inventors, using partial enrichment of PMN-MDSC with gradient centrifugation, determined that low density PMNMDSC and high-density neutrophils from the same cancer patients had a distinct gene profile.

Most prominent changes were observed in the expression of genes associated with endoplasmic reticulum (ER) stress. Surprisingly, low-density lipoprotein (LDL) was one of the most increased regulators and its receptor oxidized LDL receptor 1 (OLR1) was one of the most overexpressed genes in PMN-MDSC. Lectin-type oxidized LDL receptor 1 (LOX-1) encoded by OLR1 was practically undetectable in neutrophils in peripheral blood of healthy donors, whereas 5-15% of total neutrophils in cancer patients and 15-50% of neutrophils in tumor tissues were LOX-1+. In contrast to their LOX-1− counterparts, LOX-1+ neutrophils had gene signature, potent immune suppressive activity, up-regulation of ER stress, and other biochemical characteristics of PMN-MDSC. Moreover, induction of ER stress in neutrophils from healthy donors up-regulated LOX-1 expression and converted these cells to suppressive PMN-MDSC. As described in the specification and examples herein by evaluating populations of PMN-MDSC and PMN from the same patients, genomic signature of PMN-MDSC and certain significant surface markers specific for these cells were identified. Induction of ER stress response was sufficient to convert neutrophils to PMN-MDSC.

These discoveries by the inventors in identifying specific markers of human PMN-MDSC associated with ER stress and lipid metabolism, permit the development of novel diagnostic and therapeutic methods and compositions for cancer.

### Definitions and Components of the Methods

Unless defined otherwise in this specification, technical and scientific terms used herein have the same meaning as commonly understood by one of ordinary skill in the fields of biology, biotechnology and molecular biology and by reference to published texts, which provide one skilled in the art with a general guide to many of the terms used in the present application. The definitions herein are provided for clarity only and are not intended to limit the claimed invention.

“Patient” or “subject” as used herein means a mammalian animal, including a human, a veterinary or farm animal, a domestic animal or pet, and animals normally used for clinical research. In one embodiment, the subject of these methods and compositions is a human.

The term “LOX-1” as used herein is a cell surface receptor, oxidized low density lipoprotein (lectin-like) receptor 1, first identified in endothelial cells as one of the main receptors for oxidized-LDL (ox-LDL)⁴⁰. Besides ox-LDL, this receptor has been shown to bind many different ligands including other modified lipoproteins, advanced glycosylation end products, aged red blood cells, apoptotic cells and activated platelets⁴⁵. LOX-1 has been involved in many different pathological conditions including atherogenesis, myocardial ischemia, hypertension, vascular diseases and thrombosis⁴⁵. Expression of LOX-1 can be induced by a wide array of stimuli including pro-inflammatory factor (TNF-α, IL-1β or IFN-γ), angiotensin II, endothelin-1, modified lipoproteins and free radicals³⁵. Engagement of LOX-1 can lead to induction of oxidative stress, apoptosis, endothelial dysfunction, fibrosis and inflammation through the activation of the NF-κB pathway. LOX-1 has also been described to play a role in tumorigenesis²⁴. Indeed, LOX-1 up-regulation has been observed during cellular transformation into cancer cell and can have a pro-oncogenic effect by activating the NF-κB pathway, by increasing DNA damage through increase ROS production and by promoting angiogenesis and cell dissemination^{24,16}.

The nucleic acid sequence for the gene encoding LOX-1 (gene name OLR1) can be found in databases such as NCBI, i.e., NCBI gene ID: 4973 or Gene sequence: Ensembl:ENSG00000173391. The LOX-1 protein sequence is found at Hugo Gene Nomenclature Committee 8133, Protein Sequence HPRD:04003. It should be understood that the term LOX-1 can also represent the receptor protein in various species, and with conservative changes in the amino acid or encoding sequences, or with other naturally occurring modifications that may vary among species and between members of the same species, as well as naturally occurring mutations thereof.

The term “cancer” or “tumor” as used herein refers to, without limitation, refers to or describes the physiological condition in mammals that is typically characterized by unregulated cell growth. By cancer as used herein is meant any form of cancer, including hematological cancers, e.g., leukemia, lymphoma, myeloma, bone marrow cancer, and epithelial cancers, including, without limitation, breast cancer, lung cancer, prostate cancer, colorectal cancer, brain cancer, endometrial cancer, esophageal cancer, stomach cancer, bladder cancer, kidney cancer, pancreatic cancer, cervical cancer, head and neck cancer, ovarian cancer, melanoma, leukemia, myeloma, lymphoma, glioma, Non-Hodgkin's lymphoma, leukemia, multiple myeloma and multidrug resistant cancer.

A “tumor” is an abnormal mass of tissue that results from excessive cell division that is uncontrolled and progressive, and is also referred to as a neoplasm. The term “tumor,” as used herein, refers to all neoplastic cell growth and proliferation, whether malignant or benign, and all pre-cancerous and cancerous cells and tissues. Whenever the term “lung cancer” is used herein, it is used as a representative cancer for demonstration of the use of the methods and compositions described herein.

“Sample” as used herein means any biological fluid or suspension or tissue from a subject, including samples that contains cells carrying the LOX-1⁺ biomarker or PMN-MDSC signature biomarkers identified herein. The sample in one embodiment contains cells that are both PMN and PMN-MDSC. The sample in one embodiment contains cells carrying one or more other biomarkers or cell surface antigens indicative of polymorphonuclear cells or neutrophils. In one embodiment, cells (neutrophils) in the sample express CD66b⁺. In another embodiment, cells (neutrophils) in the sample express CD15⁺. In still another embodiment, cells in the sample express CD11b⁺ or CD33⁺. The most suitable samples for use in the methods and with the diagnostic compositions or reagents described herein are samples or suspensions which require minimal invasion for testing, e.g., blood samples, including whole blood, and any samples containing shed or circulating tumor cells. It is anticipated that other biological samples that contain cells at a sufficiently detectable concentration, such as peripheral blood, serum, saliva or urine, vaginal or cervical secretions, and ascites fluids or peritoneal fluid may be similarly evaluated by the methods described herein. In one embodiment, the sample is a tumor secretome, i.e., any fluid or medium containing the proteins secreted from the tumor. These shed proteins may be unassociated, associated with other biological molecules, or enclosed in a lipid membrane such as an exosome. Also, circulating tumor cells or fluids or tissues containing them are also suitable samples for evaluation in certain embodiments of this invention. In another embodiment, the biological sample is a tissue or tissue extract, e.g., biopsied material, containing the PMN-MDSC. In one embodiment, such samples may further be diluted with or suspended in, saline, buffer or a physiologically acceptable diluent. Alternatively, such samples are tested neat. In another embodiment, the samples are concentrated by conventional means.

In one embodiment, the biological sample is whole blood, and the method employs the PaxGene Blood RNA Workflow system (Qiagen). That system involves blood collection (e.g., single blood draws) and RNA stabilization, followed by transport and storage, followed by purification of Total RNA and Molecular RNA testing. This system provides immediate RNA stabilization and consistent blood draw volumes. The blood can be drawn at a physician's office or clinic, and the specimen transported and stored in the same tube. Short term RNA stability is 3 days at between 18-25° C. or 5 days at between 2-8° C. Long term RNA stability is 4 years at −20 to −70° C. This sample collection system enables the user to reliably obtain data on gene expression and miRNA expression in whole blood. In one embodiment, the biological sample is whole blood. While the PAXgene system has more noise than the use of PBMC as a biological sample source, the benefits of PAXgene sample collection outweighs the problems. Noise can be subtracted bioinformatically.

By “ER stress response” is meant a response mediated by the endoplasmic reticulum to protect cells from various stress conditions including hypoxia, nutrient deprivation, low pH, etc. and includes three major signaling cascades initiated by three protein sensors: PERK (protein kinase RNA (PKR)-like ER kinase), IRE-1 (inositol-requiring enzyme 1) and ATF6 (activating transcription factor 6)¹⁷. Antagonists or inhibitors of ER stress include ligands, e.g., antibodies, fragments thereof, small molecules that can block the activity, function or activation of the regulators of ER stress, identified herein.

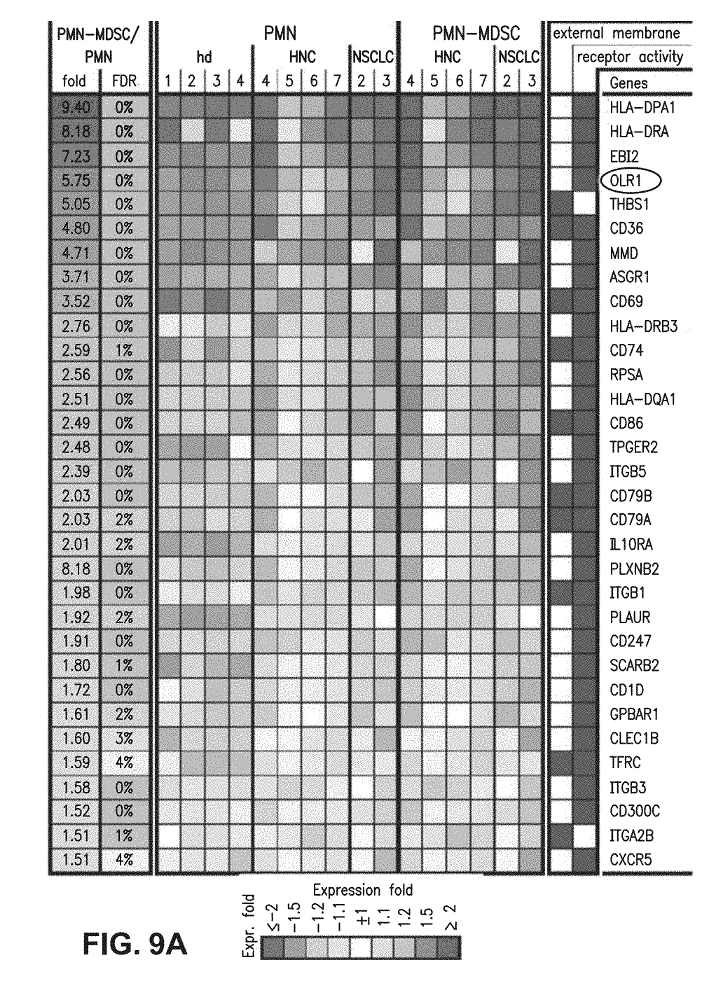

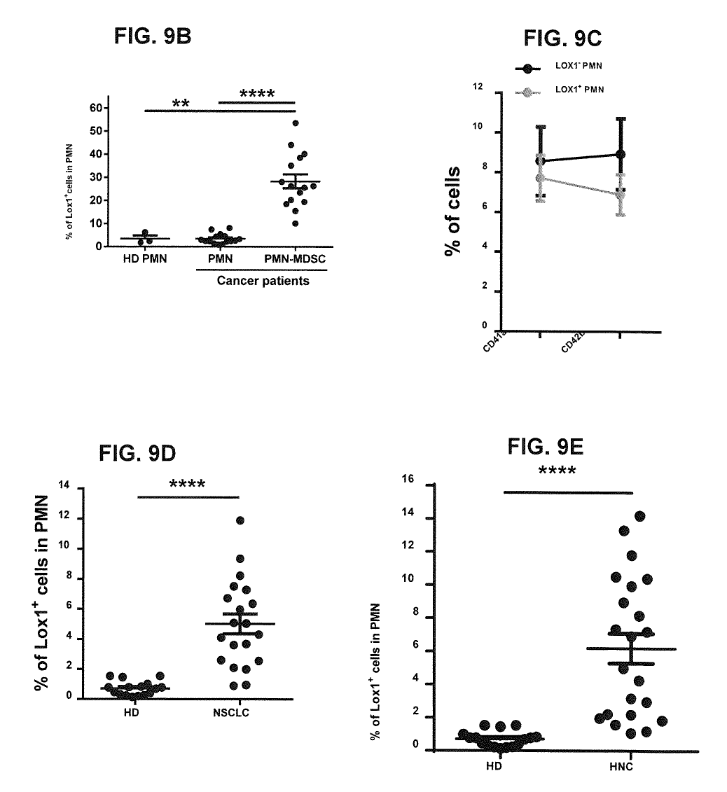

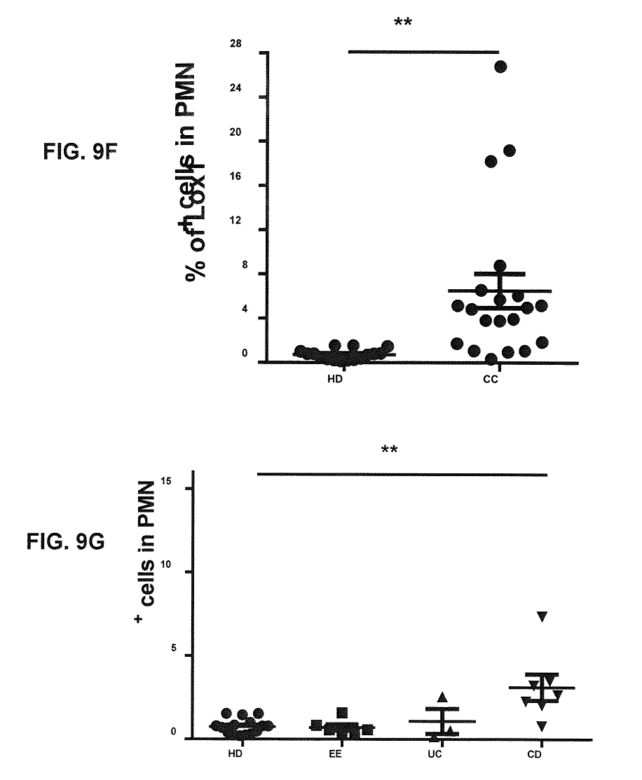

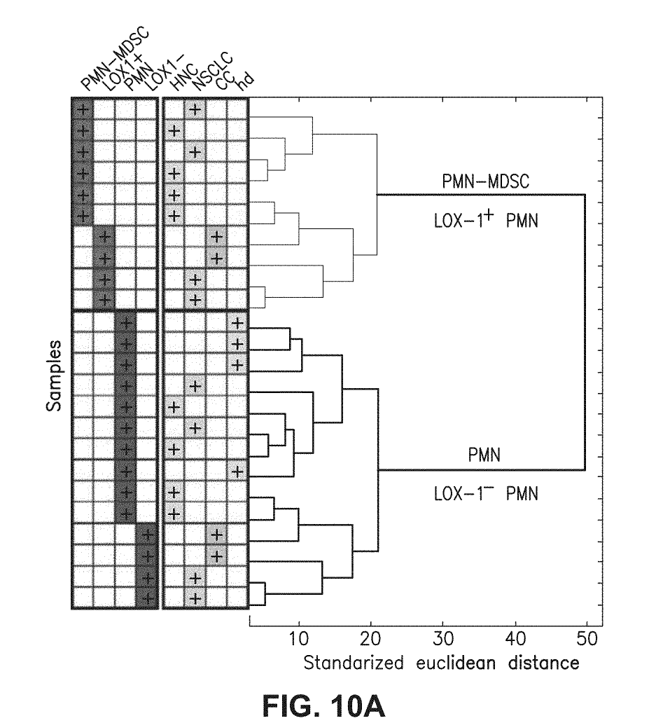

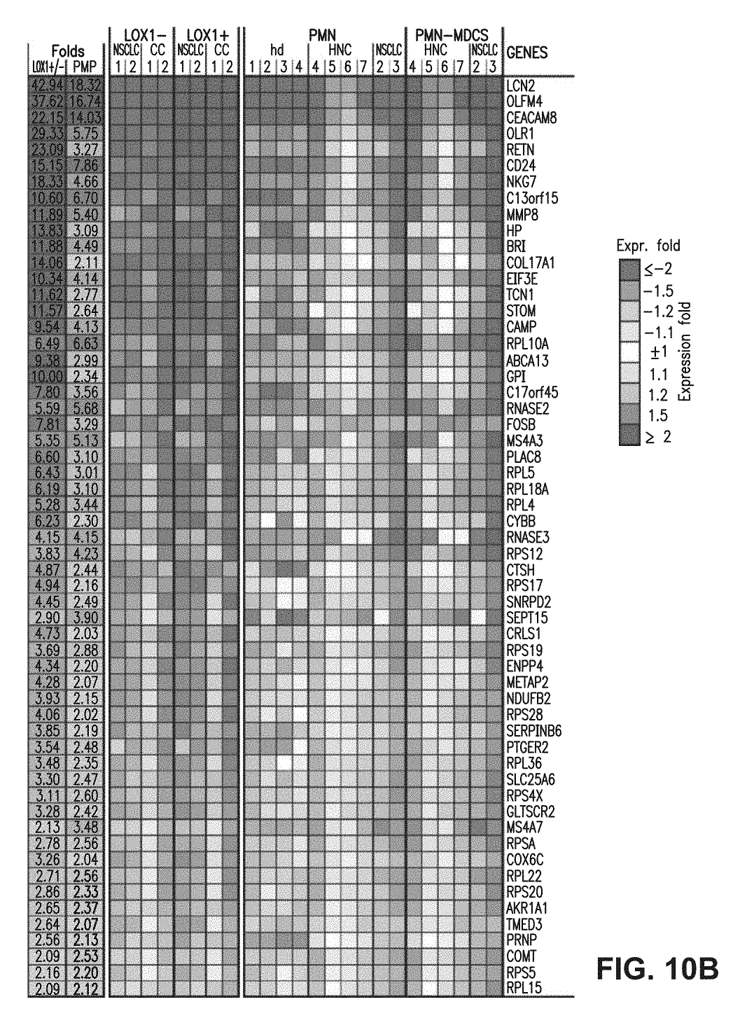

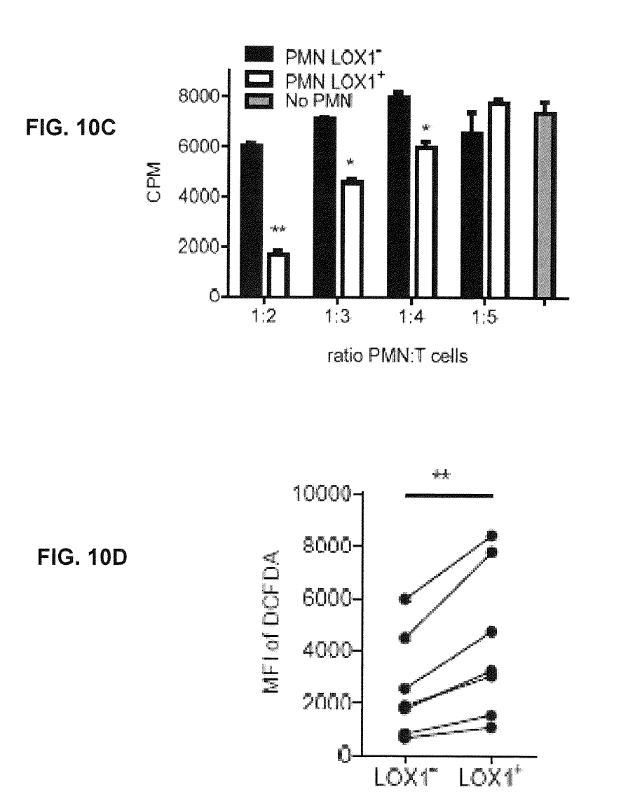

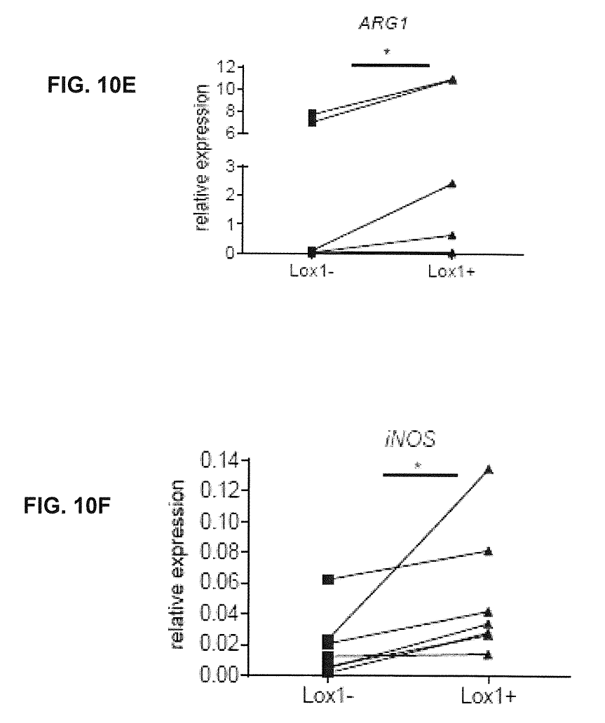

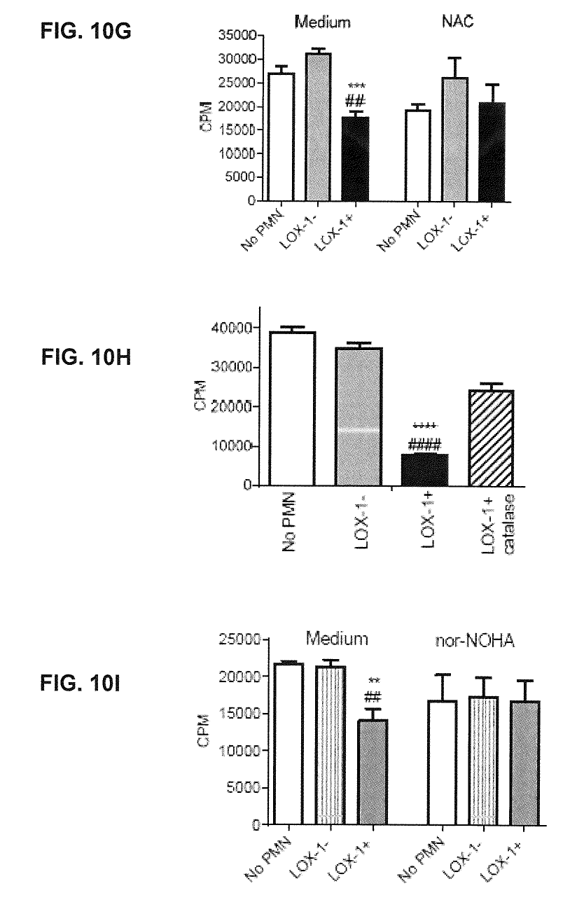

The term “biomarker” as described in this specification includes any physiological molecular form, or modified physiological molecular form, isoform, pro-form, naturally occurring forms or naturally occurring mutated forms of LOX-1 and peptide fragments of LOX-1, expressed on the cell surface, unless otherwise specified. Other biomarkers that may be useful to detect neutrophils to assist in distinguishing the two subsets PMN and PMN-MDSCs according to the teachings herein include CD66b, CD11b, CD33, CD15 and/or CD14 as well as the biomarkers that have been shown to be part of the PMN-MDSC signature, e.g., those of FIG. 9A, FIG. 10B and Table 1. It is understood that all molecular forms useful in this context are physiological, e.g., naturally occurring in the species. Preferably the peptide fragments obtained from the biomarkers are unique sequences. However, it is understood that other unique fragments may be obtained readily by one of skill in the art in view of the teachings provided herein.

By “PMN-MDSC gene signature”, as discovered by the inventors and as used throughout this specification, is meant a compilation of genes whose expression differs significantly when comparing the expression in normal neutrophils to their respective expression in PMN-MCSC, i.e., PMNs to PMN-MDSC. For example, FIG. 9A shows surface markers specific to the PMN-MDSCs and forming part of the PMN-MDSC gene signature: namely HLA-DPA1, HLA-DRA, EBI2, OLR1 THBS1, CD36, MMD, ASGR1, CD69, HLA-DRB3, CD74, RPSA, HLA-DQA1, CD86, PTGER2, ITGB5, CD79B, CD79A, IL10RA, PLXNB2, ITGB1, PLAUR, CD247, SCARB2, CD1D, GPBAR1, CLEC1B, TFRC, ITGB3, CD300C, ITGA22B and CXCR5. FIG. 10B shows genes differentially expressed between LOX-1+ and LOX-1− PMN. The following Table 1 shows the top 985 genes in rank order that can be used in various combinations to create a diagnostic signature that distinguishes PMN-MCSCs from PMN and form a PMN-MDSC gene signature. Multiple combinations of from at least 1 to all 985 of these genes, taken together, can form a PMN-MDSC signature or partial signature. Specifically, at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980 or more of the genes in Table 1 can form a PMN-MDSC signature.

TABLE 1

| Rank | Gene | Gene ID |
|---|---|---|
| 1 | defensin, alpha 1B (DEFA1B), mRNA. (S) | DEFA1B |
| 2 | defensin, alpha 3, neutrophil-specific (DEFA3), mRNA. (S) | DEFA3 |
| 3 | carcinoembryonic antigen-related cell adhesion molecule 8 | CEACAM8 |
| | (CEACAM8), mRNA. (S) | |
| 4 | PREDICTED: similar to Neutrophil defensin 1 precursor | LOC653600 |
| | (HNP-1) (HP-1) (HP1) (Defensin, alpha 1) (LOC653600), | |
| | mRNA. (S) | |
| 5 | versican (VCAN), mRNA. (S) | VCAN |
| 6 | carboxypeptidase, vitellogenic-like (CPVL), transcript | CPVL |
| | variant 1, mRNA. (A) | |
| 7 | pro-platelet basic protein (chemokine (C—X—C motif) ligand | PPBP |
| | 7) (PPBP), mRNA. (S) | |
| 8 | major histocompatibility complex, class II, DP alpha 1 | HLA-DPA1 |
| | (HLA-DPA1), mRNA. (S) | |
| 9 | defensin, alpha 4, corticostatin (DEFA4), mRNA. (S) | DEFA4 |
| 10 | major histocompatibility complex, class II, DR alpha (HLA- | HLA-DRA |
| | DRA), mRNA. (S) | |
| 11 | CD24 molecule (CD24), mRNA. (S) | CD24 |
| 12 | tubulin, beta 1 (TUBB1), mRNA. (S) | TUBB1 |
| 13 | Epstein-Barr virus induced gene 2 (lymphocyte-specific G | EBI2 |
| | protein-coupled receptor) (EBI2), mRNA. (S) | |
| 14 | annexin A2 (ANXA2), transcript variant 1, mRNA. (A) | ANXA2 |
| 15 | carboxypeptidase, vitellogenic-like (CPVL), transcript | CPVL |
| | variant 2, mRNA. (A) | |
| 16 | chromosome 13 open reading frame 15 (C13orf15), mRNA. | C13orf15 |
| | (S) | |
| 17 | S100 calcium binding protein A10 (S100A10), mRNA. (S) | S100A10 |
| 18 | ribosomal protein L10a (RPL10A), mRNA. (S) | RPL10A |
| 19 | AHNAK nucleoprotein (AHNAK), transcript variant 1, | AHNAK |
| | mRNA. (I) | |
| 20 | oxidized low density lipoprotein (lectin-like) receptor 1 | OLR1 |
| | (OLR1), mRNA. (S) | |
| 21 | lactotransferrin (LTF), mRNA. (S) | LTF |
| 22 | dihydropyrimidinase-like 2 (DPYSL2), mRNA. (S) | DPYSL2 |
| 23 | ribonuclease, RNase A family, 2 (liver, eosinophil-derived | RNASE2 |
| | neurotoxin) (RNASE2), mRNA. (S) | |
| 24 | defensin, alpha 1B (DEFA1B), mRNA. (S) | DEFA1B |
| 25 | major histocompatibility complex, class II, DR beta 6 | HLA-DRB6 |
| | (pseudogene) (HLA-DRB6), non-coding RNA. (S) | |
| 26 | matrix metallopeptidase 8 (neutrophil collagenase) (MMP8), | MMP8 |
| | mRNA. (S) | |
| 27 | early growth response 1 (EGR1), mRNA. (S) | EGR1 |
| 28 | PREDICTED: misc_RNA (LOC647276), miscRNA. (A) | LOC647276 |
| 29 | S100 calcium binding protein A10 (annexin II ligand, | S100A10 |
| | calpactin I, light polypeptide (p11)) (S100A10), mRNA. (S) | |
| 30 | membrane-spanning 4-domains, subfamily A, member 3 | MS4A3 |
| | (hematopoietic cell-specific) (MS4A3), transcript variant 1, | |
| | mRNA. (A) | |
| 31 | transforming growth factor, beta-induced, 68 kDa (TGFBI), | TGFBI |
| | mRNA. (S) | |
| 32 | thrombospondin 1 (THBS1), mRNA. (S) | THBS1 |
| 33 | PREDICTED: misc_RNA (LOC647276), miscRNA. (M) | LOC647276 |
| 34 | neurogranin (protein kinase C substrate, RC3) (NRGN), | NRGN |
| | mRNA. (S) | |
| 35 | CD36 molecule (thrombospondin receptor) (CD36), | CD36 |
| | transcript variant 1, mRNA. (I) | |
| 36 | carcinoembryonic antigen-related cell adhesion molecule 6 | CEACAM6 |
| | (non-specific cross reacting antigen) (CEACAM6), mRNA. | |
| | (S) | |
| 37 | cathepsin G (CTSG), mRNA. (S) | CTSG |
| 38 | monocyte to macrophage differentiation-associated (MMD), | MMD |
| | mRNA. (S) | |
| 39 | natural killer cell group 7 sequence (NKG7), mRNA. (S) | NKG7 |
| 40 | bactericidal/permeability-increasing protein (BPI), mRNA. | BPI |
| | (S) | |
| 41 | DNA (cytosine-5-)-methyltransferase 1 (DNMT1), mRNA. | DNMT1 |
| | (S) | |
| 42 | chromosome 21 open reading frame 7 (C21orf7), mRNA. | C21orf7 |
| | (S) | |
| 43 | PREDICTED: similar to ribosomal protein S12 | LOC651894 |
| | (LOC651894), mRNA. (S) | |
| 44 | PREDICTED: similar to 60S ribosomal protein L22 | LOC646200 |
| | (Heparin binding protein HBp15), transcript variant 1 | |
| | (LOC646200), mRNA. (A) | |
| 45 | elastase, neutrophil expressed (ELANE), mRNA. (S) | ELANE |
| 46 | guanine nucleotide binding protein (G protein), gamma 11 | GNG11 |
| | (GNG11), mRNA. (S) | |
| 47 | cat eye syndrome chromosome region, candidate 1 | CECR1 |
| | (CECR1), transcript variant 2, mRNA. (A) | |
| 48 | PREDICTED: misc_RNA (LOC644464), miscRNA. (M) | LOC644464 |
| 49 | ribosomal protein S12 (RPS12), mRNA. (S) | RPS12 |
| 50 | cyclin-dependent kinase inhibitor 1A (p21, Cip1) | CDKN1A |
| | (CDKN1A), transcript variant 1, mRNA. (A) | |
| 51 | ribonuclease, RNase A family, 3 (eosinophil cationic | RNASE3 |
| | protein) (RNASE3), mRNA. (S) | |
| 52 | eukaryotic translation initiation factor 3, subunit E (EIF3E), | EIF3E |
| | mRNA. (S) | |
| 53 | cathelicidin antimicrobial peptide (CAMP), mRNA. (S) | CAMP |
| 54 | CD36 molecule (thrombospondin receptor) (CD36), | CD36 |
| | transcript variant 3, mRNA. (A) | |
| 55 | similar to Laminin receptor 1 (LOC388524), mRNA. (S) | LOC388524 |
| 56 | PREDICTED: misc_RNA (LOC646688), miscRNA. (A) | LOC646688 |
| 57 | PREDICTED: misc_RNA (LOC391777), miscRNA. (M) | LOC391777 |
| 58 | septin 5 (SEPT5), mRNA. (S) | Sep. 5, 2014 |
| 59 | CD81 molecule (CD81), mRNA. (S) | CD81 |
| 60 | PREDICTED: hypothetical LOC730415, transcript variant 2 | LOC730415 |
| | (LOC730415), mRNA. (A) | |
| 61 | glycoprotein IX (platelet) (GP9), mRNA. (S) | GP9 |
| 62 | major histocompatibility complex, class II, DM beta (HLA- | HLA-DMB |
| | DMB), mRNA. (S) | |
| 63 | asialoglycoprotein receptor 1 (ASGR1), mRNA. (S) | ASGR1 |
| 64 | PREDICTED: similar to ribosomal protein L10a | LOC10012- |
| | (LOC100128936), mRNA. (M) | 8936 |
| 65 | PREDICTED: misc_RNA (LOC730004), miscRNA. (M) | LOC730004 |
| 66 | PREDICTED: similar to hCG1818387 (LOC391370), | LOC391370 |
| | mRNA. (M) | |
| 67 | PREDICTED: misc_RNA (LOC653162), miscRNA. (A) | LOC653162 |
| 68 | Kruppel-like factor 4 (gut) (KLF4), mRNA. (S) | KLF4 |
| 69 | PREDICTED: misc_RNA (LOC728244), miscRNA. (S) | LOC728244 |
| 70 | chromosome 17 open reading frame 45 (C17orf45), mRNA. | C17orf45 |
| | (S) | |
| 71 | ribosomal protein L18a (RPL18A), mRNA. (S) | RPL18A |
| 72 | CD69 molecule (CD69), mRNA. (S) | CD69 |
| 73 | PREDICTED: misc_RNA (LOC728553), miscRNA. (A) | LOC728553 |
| 74 | membrane-spanning 4-domains, subfamily A, member 7 | MS4A7 |
| | (MS4A7), transcript variant 2, mRNA. (A) | |
| 75 | similar to ribosomal protein L23A (LOC647099), mRNA. | LOC647099 |
| | (S) | |
| 76 | ribosomal protein L4 (RPL4), mRNA. (S) | RPL4 |
| 77 | ribosomal protein L18a (RPL18A), mRNA. (S) | RPL18A |
| 78 | PREDICTED: similar to mCG7611 (LOC284230), mRNA. | LOC284230 |
| | (M) | |
| 79 | coagulation factor XIII, A1 polypeptide (F13A1), mRNA. | F13A1 |
| | (S) | |
| 80 | FBJ murine osteosarcoma viral oncogene homolog B | FOSB |
| | (FOSB), mRNA. (S) | |
| 81 | PREDICTED: misc_RNA (LOC440027), miscRNA. (M) | LOC440027 |
| 82 | PREDICTED: misc_RNA (LOC649076), miscRNA. (S) | LOC649076 |
| 83 | resistin (RETN), mRNA. (S) | RETN |
| 84 | microRNA 1978 (MIR1978), microRNA. (S) | MIR1978 |
| 85 | Epstein-Barr virus induced gene 2 (lymphocyte-specific G | EBI2 |
| | protein-coupled receptor) (EBI2), mRNA. (S) | |
| 86 | PREDICTED: misc_RNA (LOC338870), miscRNA. (M) | LOC338870 |
| 87 | PREDICTED: similar to ribosomal protein L23a | LOC389101 |
| | (LOC389101), mRNA. (A) | |
| 88 | chemokine (C-C motif) ligand 5 (CCL5), mRNA. (S) | CCL5 |
| 89 | myeloperoxidase (MPO), nuclear gene encoding | MPO |
| | mitochondrial protein, mRNA. (S) | |
| 90 | lipase A, lysosomal acid, cholesterol esterase (LIPA), | LIPA |
| | transcript variant 2, mRNA. (S) | |
| 91 | annexin A2 pseudogene 1 (ANXA2P1) on chromosome 4. | ANXA2P1 |
| | (S) | |
| 92 | placenta-specific 8 (PLAC8), mRNA. (S) | PLAC8 |
| 93 | ribosomal protein L18a (RPL18A), mRNA. (S) | RPL18A |
| 94 | ribosomal protein L23a-like (LOC729617), mRNA. (S) | LOC729617 |
| 95 | haptoglobin (HP), mRNA. (S) | HP |
| 96 | perilipin 2 (PLIN2), mRNA. (S) | PLIN2 |
| 97 | PREDICTED: misc_RNA (LOC644464), miscRNA. (A) | LOC644464 |
| 98 | serum deprivation response (phosphatidylserine binding | SDPR |
| | protein) (SDPR), mRNA. (S) | |
| 99 | similar to ribosomal protein S18 (LOC730754), mRNA. (S) | LOC730754 |
| 100 | ribosomal protein L5 (RPL5), mRNA. (S) | RPL5 |
| 101 | ATP-binding cassette, sub-family A (ABC1), member 13 | ABCA13 |
| | (ABCA13), mRNA. (S) | |
| 102 | PREDICTED: similar to 60S ribosomal protein L23a | LOC652071 |
| | (LOC652071), mRNA. (S) | |
| 103 | PREDICTED: misc_RNA (LOC728126), miscRNA. (A) | LOC728126 |
| 104 | phosphoprotein enriched in astrocytes 15 (PEA15), mRNA. | PEA15 |
| | (S) | |
| 105 | coiled-coil domain containing 109B (CCDC109B), mRNA. | CCDC109B |
| | (S) | |
| 106 | ribosomal protein S8 (RPS8), mRNA. (S) | RPS8 |
| 107 | interferon regulatory factor 8 (IRF8), mRNA. (S) | IRF8 |
| 108 | major histocompatibility complex, class II, DM alpha | HLA-DMA |
| | (HLA-DMA), mRNA. (S) | |
| 109 | FYVE, RhoGEF and PH domain containing 2 (FGD2), | FGD2 |
| | mRNA. (S) | |
| 110 | PREDICTED: similar to 40S ribosomal protein SA (p40) | LOC647856 |
| | (34/67 kDa laminin receptor) (Colon carcinoma laminin- | |
| | binding protein) (NEM/1CHD4) (Multidrug resistance- | |
| | associated protein MGr1-Ag) (LOC647856), mRNA. (S) | |
| 111 | ribosomal protein S19 (RPS19), mRNA. (S) | RPS19 |
| 112 | major histocompatibility complex, class II, DR beta 6 | HLA-DRB6 |
| | (pseudogene) (HLA-DRB6), non-coding RNA. (S) | |
| 113 | PREDICTED: similar to 60S ribosomal protein L10 (QM | LOC644039 |
| | protein) (Tumor suppressor QM) (Laminin receptor | |
| | homolog) (LOC644039), mRNA. (S) | |
| 114 | lymphocyte antigen 86 (LY86), mRNA. (S) | LY86 |
| 115 | nuclear receptor subfamily 4, group A, member 3 (NR4A3), | NR4A3 |
| | transcript variant 4, mRNA. (I) | |
| 116 | DNA-damage-inducible transcript 4 (DDIT4), mRNA. (S) | DDIT4 |
| 117 | PREDICTED: similar to ribosomal protein S23 | LOC653658 |
| | (LOC653658), mRNA. (S) | |
| 118 | Enah/Vasp-like (EVL), mRNA. (S) | EVL |
| 119 | regulator of chromosome condensation 2 (RCC2), mRNA. | RCC2 |
| | (S) | |
| 120 | transcobalamin I (vitamin B12 binding protein, R binder | TCN1 |
| | family) (TCN1), mRNA. (S) | |
| 121 | major histocompatibility complex, class II, DR beta 3 | HLA-DRB3 |
| | (HLA-DRB3), mRNA. (S) | |
| 122 | ribosomal protein L35 (RPL35), mRNA. (S) | RPL35 |
| 123 | chemokine (C-C motif) ligand 5 (CCL5), mRNA. (S) | CCL5 |
| 124 | ribosomal protein S4, X-linked (RPS4X), mRNA. (S) | RPS4X |
| 125 | PREDICTED: misc_RNA (LOC646688), miscRNA. (M) | LOC646688 |
| 126 | salt-inducible kinase 1 (SIK1), mRNA. (S) | SIK1 |
| 127 | PREDICTED: misc_RNA (LOC645715), miscRNA. (A) | LOC645715 |
| 128 | jun oncogene (JUN), mRNA. (S) | JUN |
| 129 | metastasis suppressor 1 (MTSS1), mRNA. (S) | MTSS1 |
| 130 | membrane-spanning 4-domains, subfamily A, member 3 | MS4A3 |
| | (hematopoietic cell-specific) (MS4A3), transcript variant 1, | |
| | mRNA. (I) | |
| 131 | PREDICTED: misc_RNA (LOC389141), miscRNA. (M) | LOC389141 |
| 132 | stomatin (STOM), transcript variant 1, mRNA. (A) | STOM |
| 133 | PREDICTED: misc_RNA (LOC389223), miscRNA. (M) | LOC389223 |
| 134 | PREDICTED: hypothetical protein LOC100130919 | LOC10013- |
| | (LOC100130919), mRNA. (A) | 0919 |
| 135 | ribosomal protein S4, X-linked (RPS4X), mRNA. (S) | RPS4X |
| 136 | chloride channel, nucleotide-sensitive, 1A (CLNS1A), | CLNS1A |
| | mRNA. (S) | |
| 137 | nuclear receptor subfamily 4, group A, member 2 (NR4A2), | NR4A2 |
| | transcript variant 1, mRNA. (A) | |
| 138 | PREDICTED: misc_RNA (LOC648294), miscRNA. (A) | LOC648294 |
| 139 | FYN oncogene related to SRC, FGR, YES (FYN), transcript | FYN |
| | variant 2, mRNA. (A) | |
| 140 | CD74 molecule, major histocompatibility complex, class II | CD74 |
| | invariant chain (CD74), transcript variant 1, mRNA. (A) | |
| 141 | PREDICTED: similar to QM protein, transcript variant 2 | LOC389342 |
| | (LOC389342), mRNA. (A) | |
| 142 | PREDICTED: misc_RNA (LOC642741), miscRNA. (M) | LOC642741 |
| 143 | cysteine-rich protein 1 (intestinal) (CRIP1), mRNA. (S) | CRIP1 |
| 144 | Kruppel-like factor 4 (gut) (KLF4), mRNA. (S) | KLF4 |
| 145 | hepatitis A virus cellular receptor 2 (HAVCR2), mRNA. (S) | HAVCR2 |
| 146 | v-maf musculoaponeurotic fibrosarcoma oncogene homolog | MAFB |
| | B (avian) (MAFB), mRNA. (S) | |
| 147 | PREDICTED: similar to QM protein, transcript variant 2 | LOC284393 |
| | (LOC284393), mRNA. (A) | |
| 148 | ribosomal protein SA (RPSA), transcript variant 1, mRNA. | RPSA |
| | (A) | |
| 149 | myotubularin related protein 11 (MTMR11), mRNA. (A) | MTMR11 |
| 150 | ribosomal protein L22 (RPL22), mRNA. (S) | RPL22 |
| 151 | CD74 molecule, major histocompatibility complex, class II | CD74 |
| | invariant chain (CD74), transcript variant 2, mRNA. (A) | |
| 152 | heparin-binding EGF-like growth factor (HBEGF), mRNA. | HBEGF |
| | (S) | |
| 153 | catechol-O-methyltransferase (COMT), transcript variant S- | COMT |
| | COMT, mRNA. (A) | |
| 154 | FLJ43093 protein (FLJ43093), mRNA. (S) | FLJ43093 |
| 155 | PREDICTED: misc_RNA (LOC440595), miscRNA. (A) | LOC440595 |
| 156 | PREDICTED: similar to eukaryotic translation elongation | LOC649150 |
| | factor 1 alpha 2 (LOC649150), mRNA. (I) | |
| 157 | PREDICTED: major histocompatibility complex, class II, | HLA-DQA1 |
| | DQ alpha 1, transcript variant 10 (HLA-DQA1), mRNA. | |
| | (A) | |
| 158 | regulator of G-protein signaling 10 (RGS10), transcript | RGS10 |
| | variant 1, mRNA. (A) | |
| 159 | PREDICTED: similar to ribosomal protein L10 | LOC285176 |
| | (LOC285176), mRNA. (A) | |
| 160 | CD86 molecule (CD86), transcript variant 2, mRNA. (A) | CD86 |
| 161 | small nuclear ribonucleoprotein D2 polypeptide 16.5 kDa | SNRPD2 |
| | (SNRPD2), transcript variant 1, mRNA. (A) | |
| 162 | prostaglandin E receptor 2 (subtype EP2), 53 kDa | PTGER2 |
| | (PTGER2), mRNA. (S) | |
| 163 | PREDICTED: similar to 60S ribosomal protein L35, | LOC441246 |
| | transcript variant 1 (LOC441246), mRNA. (A) | |
| 164 | solute carrier family 25 (mitochondrial carrier; adenine | SLC25A6 |
| | nucleotide translocator), member 6 (SLC25A6), nuclear | |
| | gene encoding mitochondrial protein, mRNA. (S) | |
| 165 | PREDICTED: similar to heterogeneous nuclear | LOC645385 |
| | ribonucleoprotein A1 (LOC645385), mRNA. (S) | |
| 166 | cathepsin H (CTSH), transcript variant 1, mRNA. (A) | CTSH |
| 167 | PREDICTED: similar to bactericidal/permeability- | LOC10013- |
| | increasing protein (LOC100134379), mRNA. (S) | 4379 |
| 168 | F-box and leucine-rich repeat protein 10 (FBXL10), | FBXL10 |
| | transcript variant 1, mRNA. (A) | |
| 169 | eukaryotic translation elongation factor 1 alpha-like 7 | EEF1AL7 |
| | (EEF1AL7), non-coding RNA. (S) | |
| 170 | PREDICTED: ATPase, Na+/K+ transporting, beta 3 | ATP1B3 |
| | polypeptide, transcript variant 2 (ATP1B3), mRNA. (A) | |
| 171 | ribosomal protein S18 (RPS18), mRNA. (S) | RPS18 |
| 172 | early growth response 2 (Krox-20 homolog, Drosophila) | EGR2 |
| | (EGR2), mRNA. (S) | |
| 173 | PREDICTED: misc_RNA (LOC391126), miscRNA. (M) | LOC391126 |
| 174 | glioma tumor suppressor candidate region gene 2 | GLTSCR2 |
| | (GLTSCR2), mRNA. (S) | |
| 175 | regulator of G-protein signaling 1 (RGS1), mRNA. (S) | RGS1 |
| 176 | hypothetical LOC550643 (LOC550643), non-coding RNA. | LOC550643 |
| | (S) | |
| 177 | PREDICTED: hypothetical protein LOC100130553 | LOC10013- |
| | (LOC100130553), mRNA. (S) | 0553 |
| 178 | cathepsin L1 (CTSL1), transcript variant 2, mRNA. (A) | CTSL1 |
| 179 | PREDICTED: similar to ribosomal protein L18a, transcript | LOC285053 |
| | variant 1 (LOC285053), mRNA. (A) | |
| 180 | integrin, beta 5 (ITGB5), mRNA. XM_944688 XM_944693 | ITGB5 |
| | (A) | |
| 181 | KIAA1598 (KIAA1598), mRNA. (S) | KIAA1598 |
| 182 | eukaryotic translation elongation factor 1 alpha 1 (EEF1A1), | EEF1A1 |
| | mRNA. (S) | |
| 183 | PREDICTED: misc_RNA (LOC441073), miscRNA. (M) | LOC441073 |
| 184 | PREDICTED: misc_RNA (LOC644604), miscRNA. (A) | LOC644604 |
| 185 | aldo-keto reductase family 1, member A1 (aldehyde | AKR1A1 |
| | reductase) (AKR1A1), transcript variant 1, mRNA. (A) | |
| 186 | PREDICTED: similar to Heterogeneous nuclear | LOC648210 |
| | ribonucleoprotein A1 (Helix-destabilizing protein) (Single- | |
| | strand RNA-binding protein) (hnRNP core protein A1) | |
| | (HDP) (LOC648210), mRNA. (S) | |
| 187 | raftlin, lipid raft linker 1 (RFTN1), mRNA. (S) | RFTN1 |
| 188 | erythrocyte membrane protein band 4.1-like 3 (EPB41L3), | EPB41L3 |
| | mRNA. (S) | |
| 189 | ribosomal protein L36 (RPL36), transcript variant 1, mRNA. | RPL36 |
| | (A) | |
| 190 | PREDICTED: misc_RNA (LOC728517), partial miscRNA. | LOC728517 |
| | (S) | |
| 191 | glucose phosphate isomerase (GPI), mRNA. (S) | GPI |
| 192 | ribosomal protein S20 (RPS20), mRNA. (S) | RPS20 |
| 193 | solute carrier family 2 (facilitated glucose/fructose | SLC2A5 |
| | transporter), member 5 (SLC2A5), mRNA. (S) | |
| 194 | cathepsin L1(CTSL1), transcript variant 1, mRNA. (A) | CTSL1 |
| 195 | ribosomal protein L37a (RPL37A), mRNA. (S) | RPL37A |
| 196 | progesterone receptor membrane component 1 (PGRMC1), | PGRMC1 |
| | mRNA. (S) | |
| 197 | PREDICTED: similar to hCG2042724 (LOC100133678), | LOC10013- |
| | partial mRNA. (S) | 3678 |
| 198 | PREDICTED: misc_RNA (LOC401537), miscRNA. (A) | LOC401537 |
| 199 | chromosome 14 open reading frame 173 (C14orf173), | C14orf173 |
| | transcript variant 2, mRNA. (A) | |
| 200 | PREDICTED: misc_RNA (LOC728672), miscRNA. (A) | LOC728672 |
| 201 | small nuclear ribonucleoprotein polypeptide F (SNRPF), | SNRPF |
| | mRNA. (S) | |
| 202 | APEX nuclease (multifunctional DNA repair enzyme) 1 | APEX1 |
| | (APEX1), transcript variant 3, mRNA. (A) | |
| 203 | cytochrome b-245, beta polypeptide (chronic granulomatous | CYBB |
| | disease) (CYBB), mRNA. (S) | |
| 204 | nucleoside phosphorylase (NP), mRNA. (S) | NP |
| 205 | eukaryotic translation elongation factor 1 alpha 1 (EEF1A1), | EEF1A1 |
| | mRNA. (S) | |
| 206 | placenta-specific 8 (PLAC8), mRNA. (S) | PLAC8 |
| 207 | aldo-keto reductase family 1, member A1 (aldehyde | AKR1A1 |
| | reductase) (AKR1A1), transcript variant 2, mRNA. (A) | |
| 208 | similar to 40S ribosomal protein S17 (LOC402057), mRNA. | LOC402057 |
| | (S) | |
| 209 | PREDICTED: similar to Heterogeneous nuclear | LOC645436 |
| | ribonucleoprotein A1 (Helix-destabilizing protein) (Single- | |
| | strand binding protein) (hnRNP core protein A1) (HDP-1) | |
| | (Topoisomerase-inhibitor suppressed) (LOC645436), | |
| | mRNA. (S) | |
| 210 | family with sequence similarity 38, member A (FAM38A), | FAM38A |
| | mRNA. (S) | |
| 211 | napsin B aspartic peptidase pseudogene (NAPSB), non- | NAPSB |
| | coding RNA. XR_001413 (S) | |
| 212 | glutathione peroxidase 1 (GPX1), transcript variant 2, | GPX1 |
| | mRNA. (A) | |
| 213 | PREDICTED: misc_RNA (LOC100133372), miscRNA. (S) | LOC10013- |
| | | 3372 |
| 214 | pyrophosphatase (inorganic) 1 (PPA1), mRNA. (S) | PPA1 |
| 215 | ribosomal protein S13 (RPS13), mRNA. (S) | RPS13 |
| 216 | PREDICTED: similar to ribosomal protein L3 | LOC653881 |
| | (LOC653881), partial mRNA. (S) | |
| 217 | peroxiredoxin 1 (PRDX1), transcript variant 2, mRNA. (A) | PRDX1 |
| 218 | eukaryotic translation elongation factor 1 alpha 1 (EEF1A1), | EEF1A1 |
| | mRNA. (S) | |
| 219 | major histocompatibility complex, class II, DR alpha (HLA- | HLA-DRA |
| | DRA), mRNA. (S) | |
| 220 | CD52 molecule (CD52), mRNA. (S) | CD52 |
| 221 | family with sequence similarity 129, member B | FAM129B |
| | (FAM129B), transcript variant 1, mRNA. (S) | |
| 222 | lectin, galactoside-binding, soluble, 1 (LGALS1), mRNA. | LGALS1 |
| | (S) | |
| 223 | ribosomal protein S5 (RPS5), mRNA. (S) | RPS5 |
| 224 | major histocompatibility complex, class II, DR beta 4 | HLA-DRB4 |
| | (HLA-DRB4), mRNA. (S) | |
| 225 | ectonucleotide pyrophosphatase/phosphodiesterase 4 | ENPP4 |
| | (putative function) (ENPP4), mRNA. (S) | |
| 226 | PREDICTED: similar to ribosomal protein S12 | LOC651894 |
| | (LOC651894), mRNA. (S) | |
| 227 | SH3 domain binding glutamic acid-rich protein like 2 | SH3BGRL2 |
| | (SH3BGRL2), mRNA. (S) | |
| 228 | ATP-binding cassette, sub-family C (CFTR/MRP), member | ABCC3 |
| | 3 (ABCC3), mRNA. (A) | |
| 229 | serpin peptidase inhibitor, clade B (ovalbumin), member 6 | SERPINB6 |
| | (SERPINB6), mRNA. (S) | |
| 230 | PREDICTED: similar to ribosomal protein L18a; 60S | LOC390354 |
| | ribosomal protein L18a, transcript variant 36 (LOC390354), | |
| | misc RNA. (A) | |
| 231 | PREDICTED: similar to Heterogeneous nuclear | LOC648210 |
| | ribonucleoprotein A1 (Helix-destabilizing protein) (Single- | |
| | strand RNA-binding protein) (hnRNP core protein A1) | |
| | (HDP) (LOC648210), mRNA. (A) | |
| 232 | tumor necrosis factor (TNF superfamily, member 2) (TNF), | TNF |
| | mRNA. (S) | |
| 233 | PREDICTED: ATPase, Na+/K+ transporting, beta 3 | ATP1B3 |
| | polypeptide, transcript variant 2 (ATP1B3), mRNA. (A) | |
| 234 | ribosomal protein S17 (RPS17), mRNA. (S) | RPS17 |
| 235 | NADH dehydrogenase (ubiquinone) 1 beta subcomplex, 2, | NDUFB2 |
| | 8 kDa (NDUFB2), nuclear gene encoding mitochondrial | |
| | protein, mRNA. (S) | |
| 236 | PREDICTED: misc_RNA (LOC728553), miscRNA. (M) | LOC728553 |
| 237 | prion protein (PRNP), transcript variant 3, mRNA. (A) | PRNP |
| 238 | eukaryotic translation elongation factor 1 beta 2 (EEF1B2), | EEF1B2 |
| | transcript variant 3, mRNA. (A) | |
| 239 | ribosomal protein, large, P1 (RPLP1), transcript variant 1, | RPLP1 |
| | mRNA. (I) | |
| 240 | PREDICTED: transaldolase 1 (TALDO1), mRNA. (I) | TALDO1 |
| 241 | cytoplasmic FMR1 interacting protein 1 (CYFIP1), | CYFIP1 |
| | transcript variant 2, mRNA. (A) | |
| 242 | chromosome 19 open reading frame 2 (C19orf2), transcript | C19orf2 |
| | variant 2, mRNA. (A) | |
| 243 | ribosomal protein L15 (RPL15), mRNA. (S) | RPL15 |
| 244 | collagen, type XVII, alpha 1 (COL17A1), mRNA. (A) | COL17A1 |
| 245 | PREDICTED: similar to 60S ribosomal protein L7 | LOC650276 |
| | (LOC650276), mRNA. (S) | |
| 246 | poly(A) binding protein, cytoplasmic 4 (inducible form) | PABPC4 |
| | (PABPC4), mRNA. (S) | |
| 247 | PREDICTED: similar to acidic ribosomal phosphoprotein | LOC649049 |
| | P0, transcript variant 3 (LOC649049), mRNA. (A) | |
| 248 | nuclear receptor subfamily 4, group A, member 3 (NR4A3), | NR4A3 |
| | transcript variant 2, mRNA. (A) | |
| 249 | PREDICTED: similar to 40S ribosomal protein S28 | LOC728453 |
| | (LOC728453), mRNA. (M) | |
| 250 | PREDICTED: similar to 60S ribosomal protein L35 | LOC646766 |
| | (LOC646766), mRNA. (S) | |
| 251 | transmembrane emp24 protein transport domain containing | TMED3 |
| | 3 (TMED3), mRNA. (S) | |
| 252 | methionyl aminopeptidase 2 (METAP2), mRNA. (S) | METAP2 |
| 253 | CXXC finger 5 (CXXC5), mRNA. (S) | CXXC5 |
| 254 | X-prolyl aminopeptidase (aminopeptidase P) 1, soluble | XPNPEP1 |
| | (XPNPEP1), mRNA. (S) | |
| 255 | F-box and leucine-rich repeat protein 18 (FBXL18), mRNA. | FBXL18 |
| | (S) | |
| 256 | PREDICTED: similar to HLA class II histocompatibility | LOC649143 |
| | antigen, DRB1-9 beta chain precursor (MHC class I antigen | |
| | DRB1*9) (DR-9) (DR9), transcript variant 2 (LOC649143), | |
| | mRNA. (A) | |
| 257 | PREDICTED: similar to ribosomal protein, transcript | LOC388339 |
| | variant 4 (LOC388339), mRNA. (M) | |
| 258 | dedicator of cytokinesis 10 (DOCK10), mRNA. (S) | DOCK10 |
| 259 | PREDICTED: similar to 40S ribosomal protein SA (p40) | LOC648249 |
| | (34/67 kDa laminin receptor) (Colon carcinoma laminin- | |
| | binding protein) (NEM/1CHD4) (Multidrug resistance- | |
| | associated protein MGr1-Ag), transcript variant 3 | |
| | (LOC648249), mRNA. (A) | |
| 260 | cytochrome c oxidase subunit VIc (COX6C), mRNA. (S) | COX6C |
| 261 | cardiolipin synthase 1 (CRLS1), mRNA. (S) | CRLS1 |
| 262 | PREDICTED: misc_RNA (LOC728576), miscRNA. (M) | LOC728576 |
| 263 | eukaryotic translation elongation factor 1 beta 2 (EEF1B2), | EEF1B2 |
| | transcript variant 1, mRNA. (A) | |
| 264 | CD79b molecule, immunoglobulin-associated beta | CD79B |
| | (CD79B), transcript variant 3, mRNA. (A) | |
| 265 | CD79a molecule, immunoglobulin-associated alpha | CD79A |
| | (CD79A), transcript variant 2, mRNA. (A) | |
| 266 | ribosomal protein S28 (RPS28), mRNA. (S) | RPS28 |
| 267 | interleukin 10 receptor, alpha (IL10RA), mRNA. (S) | IL10RA |
| 268 | chromosome 4 open reading frame 18 (C4orf18), transcript | C4orf18 |
| | variant 2, mRNA. (I) | |
| 269 | peroxiredoxin 1 (PRDX1), transcript variant 2, mRNA. (A) | PRDX1 |
| 270 | anoctamin 6 (ANO6), mRNA. (S) | ANO6 |
| 271 | PREDICTED: similar to major histocompatibility complex, | LOC10013- |
| | class II, DQ beta 1, transcript variant 2 (LOC100133583), | 3583 |
| | mRNA. (A) | |
| 272 | PREDICTED: misc_RNA (LOC441506), miscRNA. (A) | LOC441506 |
| 273 | PREDICTED: similar to laminin receptor 1 (ribosomal | LOC388654 |
| | protein SA) (LOC388654), mRNA. (S) | |
| 274 | ribosomal protein L3 (RPL3), transcript variant 2, mRNA. | RPL3 |
| | (A) | |
| 275 | ribosomal protein, large, P2 (RPLP2), mRNA. (S) | RPLP2 |
| 276 | plexin B2 (PLXNB2), mRNA. (A) | PLXNB2 |
| 277 | ribosomal protein L6 (RPL6), transcript variant 1, mRNA. | RPL6 |
| | (A) | |
| 278 | Rho GTPase activating protein 21 (ARHGAP21), mRNA. | ARHGAP21 |
| | (S) | |
| 279 | cytochrome c oxidase subunit VIIc (COX7C), nuclear gene | COX7C |
| | encoding mitochondrial protein, mRNA. (S) | |
| 280 | N-acetylglucosamine-1-phosphodiester alpha-N- | NAGPA |
| | acetylglucosaminidase (NAGPA), mRNA. (S) | |
| 281 | hypothetical protein MGC13057 (MGC13057), mRNA. (S) | MGC13057 |
| 282 | PREDICTED: misc_RNA (LOC100129158), miscRNA. | LOC10012- |
| | (M) | 9158 |
| 283 | lectin, galactoside-binding, soluble, 3 (galectin 3) | LGALS3 |
| | (LGALS3), mRNA. (S) | |
| 284 | phospholipid scramblase 3 (PLSCR3), mRNA. (S) | PLSCR3 |
| 285 | integrin, beta 1 (fibronectin receptor, beta polypeptide, | ITGB1 |
| | antigen CD29 includes MDF2, MSK12) (ITGB1), transcript | |
| | variant 1A, mRNA. (I) | |
| 286 | asialoglycoprotein receptor 2 (ASGR2), transcript variant 3, | ASGR2 |
| | mRNA. (A) | |
| 287 | PREDICTED: misc_RNA (LOC645387), miscRNA. (M) | LOC645387 |
| 288 | vesicle-associated membrane protein 8 (endobrevin) | VAMP8 |
| | (VAMP8), mRNA. (S) | |
| 289 | PREDICTED: similar to eukaryotic translation elongation | LOC402251 |
| | factor 1 alpha 2 (LOC402251), mRNA. (S) | |
| 290 | peptidase D (PEPD), mRNA. (S) | PEPD |
| 291 | PREDICTED: hypothetical protein LOC100131831 | LOC10013- |
| | (LOC100131831), mRNA. (M) | 1831 |
| 292 | peptidylprolyl isomerase A processed pseudogene | LOC134997 |
| | (LOC134997) on chromosome 6. (S) | |
| 293 | PREDICTED: similar to golgi autoantigen, golgin subfamily | LOC653061 |
| | a, 8A (LOC653061), mRNA. (S) | |
| 294 | interleukin enhancer binding factor 2, 45 kDa (ILF2), | ILF2 |
| | mRNA. (S) | |
| 295 | PREDICTED: misc_RNA (LOC729102), miscRNA. (M) | LOC729102 |
| 296 | CD24 molecule (CD24), mRNA. (S) | CD24 |
| 297 | PREDICTED: similar to Elongation factor 1-gamma (EF-1- | LOC731096 |
| | gamma) (eEF-1B gamma) (LOC731096), mRNA. (A) | |
| 298 | fibrillarin (FBL), mRNA. (S) | FBL |
| 299 | ribosomal protein L3 (RPL3), transcript variant 2, mRNA. | RPL3 |
| | (A) | |
| 300 | PREDICTED: similar to 40S ribosomal protein SA (p40) | LOC387867 |
| | (34/67 kDa laminin receptor) (Colon carcinoma laminin- | |
| | binding protein) (NEM/1CHD4) (Multidrug resistance- | |
| | associated protein MGr1-Ag) (LOC387867), mRNA. (A) | |
| 301 | phosphatidylserine synthase 1 (PTDSS1), mRNA. (S) | PTDSS1 |
| 302 | PREDICTED: similar to Ribosomal protein L6, transcript | LOC641814 |
| | variant 7 (LOC641814), mRNA. (S) | |
| 303 | small nuclear ribonucleoprotein polypeptide F (SNRPF), | SNRPF |
| | mRNA. (S) | |
| 304 | ribosomal protein L12 (RPL12), mRNA. (S) | RPL12 |
| 305 | CTD (carboxy-terminal domain, RNA polymerase II, | CTDSPL |
| | polypeptide A) small phosphatase-like (CTDSPL), transcript | |
| | variant 2, mRNA. (A) | |
| 306 | PREDICTED: misc_RNA (LOC439953), miscRNA. (A) | LOC439953 |
| 307 | PREDICTED: similar to Heterogeneous nuclear | LOC648210 |
| | ribonucleoprotein A1 (Helix-destabilizing protein) (Single- | |
| | strand RNA-binding protein) (hnRNP core protein A1) | |
| | (HDP) (LOC648210), mRNA. (A) | |
| 308 | brain expressed, X-linked 1 (BEX1), mRNA. (S) | BEX1 |
| 309 | COMM domain containing 7 (COMMD7), transcript variant | COMMD7 |
| | 2, mRNA. (S) | |
| 310 | activating transcription factor 3 (ATF3), transcript variant 4, | ATF3 |
| | mRNA. (A) | |
| 311 | PREDICTED: hypothetical LOC388076 (LOC388076), | LOC388076 |
| | mRNA. (A) | |
| 312 | ribosomal protein L19 (RPL19), mRNA. (S) | RPL19 |
| 313 | plasminogen activator, urokinase receptor (PLAUR), | PLAUR |
| | transcript variant 1, mRNA. (A) | |
| 314 | ribosomal protein L6 (RPL6), transcript variant 1, mRNA. | RPL6 |
| | (A) | |
| 315 | PREDICTED: similar to 60S ribosomal protein L7a | LOC644029 |
| | (LOC644029), mRNA. (A) | |
| 316 | aldehyde dehydrogenase 2 family (mitochondrial) | ALDH2 |
| | (ALDH2), nuclear gene encoding mitochondrial protein, | |
| | mRNA. (S) | |
| 317 | PREDICTED: similar to ribosomal protein L23a | LOC729798 |
| | (LOC729798), mRNA. (M) | |
| 318 | ribosomal protein L18 (RPL18), mRNA. (S) | RPL18 |
| 319 | PREDICTED: hypothetical protein MGC16384 | MGC16384 |
| | (MGC16384), mRNA. (S) | |
| 320 | TSC22 domain family, member 1 (TSC22D1), transcript | TSC22D1 |
| | variant 2, mRNA. (A) | |
| 321 | PREDICTED: similar to 40S ribosomal protein S28 | LOC645899 |
| | (LOC645899), mRNA. (S) | |
| 322 | PREDICTED: misc_RNA (LOC728576), miscRNA. (A) | LOC728576 |
| 323 | ribosomal protein L32 (RPL32), transcript variant 3, mRNA. | RPL32 |
| | (S) | |
| 324 | ribosomal protein S6 (RPS6), mRNA. (S) | RPS6 |
| 325 | CD247 molecule (CD247), transcript variant 1, mRNA. (A) | CD247 |
| 326 | mitogen-activated protein kinase kinase kinase 8 | MAP3K8 |
| | (MAP3K8), mRNA. (S) | |
| 327 | integrin, beta 5 (ITGB5), mRNA. XM_944688 XM_944693 | ITGB5 |
| | (A) | |
| 328 | eukaryotic translation initiation factor 3, subunit B (EIF3B), | EIF3B |
| | transcript variant 2, mRNA. (A) | |
| 329 | PREDICTED: similar to ribosomal protein L29, transcript | LOC10013- |
| | variant 2 (LOC100131713), mRNA. (A) | 1713 |
| 330 | ribosomal protein, large, P1 (RPLP1), transcript variant 2, | RPLP1 |
| | mRNA. (A) | |
| 331 | ribosomal protein L8 (RPL8), transcript variant 2, mRNA. | RPL8 |
| | (A) | |
| 332 | chromosome 20 open reading frame 27 (C20orf27), mRNA. | C20orf27 |
| | (S) | |
| 333 | PREDICTED: similar to 60S ribosomal protein L7, | LOC648000 |
| | transcript variant 1 (LOC648000), mRNA. (A) | |
| 334 | CD69 molecule (CD69), mRNA. (S) | CD69 |
| 335 | ribosomal protein L13a (RPL13A), mRNA. (S) | RPL13A |
| 336 | zinc finger, DHHC-type containing 8 (ZDHHC8), mRNA. | ZDHHC8 |
| | (S) | |
| 337 | PREDICTED: similar to ribosomal protein S2, transcript | LOC440589 |
| | variant 3 (LOC440589), mRNA. (A) | |
| 338 | PREDICTED: misc_RNA (LOC100129424), miscRNA. (A) | LOC10012- |
| | | 9424 |
| 339 | v-fos FBJ murine osteosarcoma viral oncogene homolog | FOS |
| | (FOS), mRNA. (S) | |
| 340 | ArfGAP with dual PH domains 2 (ADAP2), mRNA. (S) | ADAP2 |
| 341 | split hand/foot malformation (ectrodactyly) type 1 | SHFM1 |
| | (SHFM1), mRNA. (S) | |
| 342 | PREDICTED: protein tyrosine phosphatase, non-receptor | PTPN20 |
| | type 20 (PTPN20), mRNA. (S) | |
| 343 | PREDICTED: misc_RNA (LOC390345), miscRNA. (M) | LOC390345 |
| 344 | E2F transcription factor 2 (E2F2), mRNA. (S) | E2F2 |
| 345 | polymerase (DNA-directed), epsilon 4 (p12 subunit) | POLE4 |
| | (POLE4), mRNA. (S) | |
| 346 | ribosomal protein L23a pseudogene (LOC649946), non- | LOC649946 |
| | coding RNA. (S) | |
| 347 | PREDICTED: similar to hCG1997137, transcript variant 3 | LOC730029 |
| | (LOC730029), mRNA. (A) | |
| 348 | PREDICTED: misc_RNA (LOC387930), miscRNA. (M) | LOC387930 |
| 349 | PREDICTED: hypothetical LOC400455 (LOC400455), | LOC400455 |
| | mRNA. (S) | |
| 350 | tensin 3 (TNS3), mRNA. (S) | TNS3 |
| 351 | PREDICTED: similar to 60S ribosomal protein L6 (TAX- | LOC285900 |
| | responsive enhancer element binding protein 107) | |
| | (TAXREB107) (Neoplasm-related protein C140), transcript | |
| | variant 3 (LOC285900), mRNA. (A) | |
| 352 | guanine nucleotide binding protein (G protein), beta | GNB2L1 |
| | polypeptide 2-like 1 (GNB2L1), mRNA. (S) | |
| 353 | synuclein, alpha (non A4 component of amyloid precursor) | SNCA |
| | (SNCA), transcript variant NACP112, mRNA. (A) | |
| 354 | PREDICTED: similar to hCG2027326 (LOC100132291), | LOC10013- |
| | mRNA. (M) | 2291 |
| 355 | membrane-spanning 4-domains, subfamily A, member 7 | MS4A7 |
| | (MS4A7), transcript variant 2, mRNA. (A) | |
| 356 | PREDICTED: similar to 60S ribosomal protein L18 | LOC441775 |
| | (LOC441775), mRNA. (S) | |
| 357 | prostaglandin-endoperoxide synthase 1 (prostaglandin G/H | PTGS1 |
| | synthase and cyclooxygenase) (PTGS1), transcript variant 2, | |
| | mRNA. (A) | |
| 358 | sulfatase modifying factor 2 (SUMF2), transcript variant 4, | SUMF2 |
| | mRNA. (A) | |
| 359 | nuclear receptor interacting protein 3 (NRIP3), mRNA. (S) | NRIP3 |
| 360 | PREDICTED: misc_RNA (LOC730246), miscRNA. (M) | LOC730246 |
| 361 | PREDICTED: misc_RNA (LOC645387), miscRNA. (A) | LOC645387 |
| 362 | PREDICTED: misc_RNA (RPL14L), miscRNA. (M) | RPL14L |
| 363 | PREDICTED: hypothetical LOC653232, transcript variant 4 | LOC653232 |
| | (LOC653232), mRNA. (A) | |
| 364 | caspase recruitment domain family, member 9 (CARD9), | CARD9 |
| | mRNA. (S) | |
| 365 | PREDICTED: misc_RNA (LOC100127993), miscRNA. | LOC10012- |
| | (M) | 7993 |
| 366 | PREDICTED: similar to 40S ribosomal protein S28, | LOC646195 |
| | transcript variant 2 (LOC646195), mRNA. (S) | |
| 367 | cysteine-rich secretory protein 3 (CRISP3), mRNA. (S) | CRISP3 |
| 368 | PREDICTED: similar to 60S ribosomal protein L32 | LOC642210 |
| | (LOC642210), mRNA. (S) | |
| 369 | CD68 antigen (CD68), mRNA. (S) | CD68 |
| 370 | PREDICTED: misc_RNA (LOC148430), miscRNA. (A) | LOC148430 |
| 371 | dehydrogenase/reductase (SDR family) member 4 (DHRS4), | DHRS4 |
| | mRNA. (S) | |
| 372 | PREDICTED: hypothetical protein LOC651309 | LOC651309 |
| | (LOC651309), mRNA. (S) | |
| 373 | nuclear receptor subfamily 4, group A, member 2 (NR4A2), | NR4A2 |
| | transcript variant 1, mRNA. (A) | |
| 374 | caspase 8, apoptosis-related cysteine peptidase (CASP8), | CASP8 |
| | transcript variant B, mRNA. (A) | |
| 375 | TGF beta-inducible nuclear protein 1 (TINP1), mRNA. (S) | TINP1 |
| 376 | lactamase, beta (LACTB), nuclear gene encoding | LACTB |
| | mitochondrial protein, transcript variant 2, mRNA. (I) | |
| 377 | heterogeneous nuclear ribonucleoprotein A1 pseudogene | HNRPA1L-2 |
| | (HNRPA1L-2), non-coding RNA. (S) | |
| 378 | PDZ and LIM domain 1 (PDLIM1), mRNA. (S) | PDLIM1 |
| 379 | thymosin beta 10 (TMSB10), mRNA. (S) | TMSB10 |
| 380 | microsomal glutathione S-transferase 1 (MGST1), transcript | MGST1 |
| | variant 1a, mRNA. (A) | |
| 381 | PREDICTED: similar to large subunit ribosomal protein | LOC651202 |
| | L36a (LOC651202), mRNA. (S) | |
| 382 | similar to ribosomal protein L15 (LOC402694), mRNA. (A) | LOC402694 |
| 383 | PREDICTED: misc_RNA (LOC648771), miscRNA. (M) | LOC648771 |
| 384 | GTP binding protein 6 (putative) (GTPBP6), mRNA. (S) | GTPBP6 |
| 385 | ribosomal protein S3A (RPS3A), mRNA. (S) | RPS3A |
| 386 | mitochondrial ribosomal protein S12 (MRPS12), nuclear | MRPS12 |
| | gene encoding mitochondrial protein, transcript variant 3, | |
| | mRNA. (A) | |
| 387 | enoyl Coenzyme A hydratase domain containing 2 | ECHDC2 |
| | (ECHDC2), mRNA. (S) | |
| 388 | PREDICTED: misc_RNA (LOC728128), miscRNA. (A) | LOC728128 |
| 389 | chromosome 4 open reading frame 18 (C4orf18), transcript | C4orf18 |
| | variant 2, mRNA. (A) | |
| 390 | non-SMC element 4 homolog A (S. cerevisiae) | NSMCE4A |
| | (NSMCE4A), mRNA. (S) | |
| 391 | tripartite motif-containing 44 (TRIM44), mRNA. (S) | TRIM44 |
| 392 | endoplasmic reticulum protein 29 (ERP29), transcript | ERP29 |
| | variant 2, mRNA. (A) | |
| 393 | similar to ribosomal protein L19 (LOC653314), mRNA. (A) | LOC653314 |
| 394 | scavenger receptor class B, member 2 (SCARB2), mRNA. | SCARB2 |
| | (S) | |
| 395 | neuron derived neurotrophic factor (NENF), mRNA. (S) | NENF |
| 396 | PREDICTED: similar to 60S ribosomal protein L32 | LOC649548 |
| | (LOC649548), mRNA. (S) | |
| 397 | retinol dehydrogenase 11 (all-trans/9-cis/11-cis) (RDH11), | RDH11 |
| | mRNA. (S) | |
| 398 | ye15f04.x5 Stratagene lung (#937210) cDNA clone | NaN |
| | IMAGE: 117823 3 similar to contains element MER6 | |
| | repetitive element;, mRNA sequence (S) | |
| 399 | oligosaccharyltransferase complex subunit (OSTC), mRNA. | OSTC |
| | (S) | |
| 400 | PREDICTED: similar to NADH dehydrogenase subunit 5 | LOC643031 |
| | (LOC643031), mRNA. (S) | |
| 401 | CD247 molecule (CD247), transcript variant 2, mRNA. (A) | CD247 |
| 402 | plasminogen activator, urokinase receptor (PLAUR), | PLAUR |
| | transcript variant 2, mRNA. (A) | |
| 403 | PREDICTED: misc_RNA (LOC729236), miscRNA. (A) | LOC729236 |
| 404 | PREDICTED: misc_RNA (LOC647030), miscRNA. (A) | LOC647030 |
| 405 | leucine rich repeat containing 33 (LRRC33), mRNA. (S) | LRRC33 |
| 406 | mitochondrial ribosomal protein L3 (MRPL3), nuclear gene | MRPL3 |
| | encoding mitochondrial protein, mRNA. (S) | |
| 407 | PREDICTED: similar to 60S ribosomal protein L23a | FLJ43681 |
| | (FLJ43681), miscRNA. (S) | |
| 408 | PREDICTED: misc_RNA (LOC728368), miscRNA. (A) | LOC728368 |
| 409 | stearoyl-CoA desaturase (delta-9-desaturase) (SCD), | SCD |
| | mRNA. (S) | |
| 410 | PREDICTED: similar to 40S ribosomal protein S4, X | LOC220433 |
| | isoform (LOC220433), mRNA. (A) | |
| 411 | PREDICTED: misc_RNA (RPL14L), miscRNA. (A) | RPL14L |
| 412 | EP300 interacting inhibitor of differentiation 3 (EID3), | EID3 |
| | mRNA. (S) | |
| 413 | NADH dehydrogenase (ubiquinone) 1 alpha subcomplex, | NDUFAF3 |
| | assembly factor 3 (NDUFAF3), nuclear gene encoding | |
| | mitochondrial protein, transcript variant 1, mRNA. (A) | |
| 414 | ribosomal protein S3A (RPS3A), mRNA. (S) | RPS3A |
| 415 | PREDICTED: similar to ribosomal protein L13a | LOC645683 |
| | (LOC645683), mRNA. (S) | |
| 416 | PREDICTED: misc_RNA (LOC642357), miscRNA. (M) | LOC642357 |
| 417 | nerve growth factor receptor (TNFRSF16) associated | NGFRAP1 |
| | protein 1 (NGFRAP1), transcript variant 1, mRNA. (A) | |
| 418 | intercellular adhesion molecule 2 (ICAM2), transcript | ICAM2 |
| | variant 1, mRNA. (S) | |
| 419 | CDC42 effector protein (Rho GTPase binding) 3 | CDC42EP3 |
| | (CDC42EP3), mRNA. (S) | |
| 420 | golgi associated, gamma adaptin ear containing, ARF | GGA2 |
| | binding protein 2 (GGA2), mRNA. (S) | |
| 421 | ribosomal protein S3A (RPS3A), mRNA. (S) | RPS3A |
| 422 | PREDICTED: similar to 23 kD highly basic protein, | LOC728658 |
| | transcript variant 1 (LOC728658), mRNA. (M) | |
| 423 | vanin 3 (VNN3), transcript variant 2, mRNA. (I) | VNN3 |
| 424 | ribosomal protein L9 (RPL9), transcript variant 2, mRNA. | RPL9 |
| | (A) | |
| 425 | CD151 molecule (Raph blood group) (CD151), transcript | CD151 |
| | variant 2, mRNA. (A) | |
| 426 | PREDICTED: similar to ribosomal protein L24 | LOC731365 |
| | (LOC731365), mRNA. (S) | |
| 427 | lectin, galactoside-binding, soluble, 2 (LGALS2), mRNA. | LGALS2 |
| | (S) | |
| 428 | S-adenosylhomocysteine hydrolase (AHCY), mRNA. (S) | AHCY |
| 429 | PREDICTED: similar to 60S ribosomal protein L29 (Cell | LOC649447 |
| | surface heparin binding protein HIP) (LOC649447), mRNA. | |
| | (S) | |
| 430 | PREDICTED: misc_RNA (LOC646294), miscRNA. (A) | LOC646294 |
| 431 | AV762101 MDS cDNA clone MDSEOA03 5, mRNA | NaN |
| | sequence (S) | |
| 432 | LSM4 homolog, U6 small nuclear RNA associated (S. cerevisiae) | LSM4 |
| | (LSM4), mRNA. (S) | |
| 433 | ribosomal protein S27 (metallopanstimulin 1) (RPS27), | RPS27 |
| | mRNA. (S) | |
| 434 | transmembrane protein 147 (TMEM147), mRNA. (S) | TMEM147 |
| 435 | centromere protein B, 80 kDa (CENPB), mRNA. (S) | CENPB |
| 436 | tetraspanin 33 (TSPAN33), mRNA. (S) | TSPAN33 |
| 437 | PREDICTED: misc_RNA (LOC645174), miscRNA. (M) | LOC645174 |
| 438 | PREDICTED: misc_RNA (LOC402112), miscRNA. (A) | LOC402112 |
| 439 | small nucleolar RNA host gene 6 (non-protein coding) | SNHG6 |
| | (SNHG6), non-coding RNA. (S) | |
| 440 | PREDICTED: misc_RNA (LOC389404), miscRNA. (A) | LOC389404 |
| 441 | PREDICTED: misc_RNA (LOC643531), miscRNA. (M) | LOC643531 |
| 442 | small nucleolar RNA host gene (non-protein coding) 5 | SNHG5 |
| | (SNHG5) on chromosome 6. (S) | |
| 443 | small nuclear ribonucleoprotein polypeptide N (SNRPN), | SNRPN |
| | transcript variant 2, mRNA. (A) | |
| 444 | PREDICTED: misc_RNA (LOC100129541), miscRNA. | LOC100129541 |
| | (M) | |
| 445 | PREDICTED: misc_RNA (LOC100132795), miscRNA. | LOC100132795 |
| | (M) | |
| 446 | CD1d molecule (CD1D), mRNA. (S) | CD1D |
| 447 | growth factor independent 1 transcription repressor (GFI1), | GFI1 |
| | mRNA. (S) | |
| 448 | eukaryotic translation elongation factor 1 gamma (EEF1G), | EEF1G |
| | mRNA. XM_935976 XM_935977 XM_935978 | |
| | XM_935979 (I) | |
| 449 | dual specificity phosphatase 2 (DUSP2), mRNA. (S) | DUSP2 |
| 450 | malate dehydrogenase 1, NAD (soluble) (MDH1), mRNA. | MDH1 |
| | (S) | |
| 451 | PREDICTED: similar to 23 kD highly basic protein, | LOC728658 |
| | transcript variant 1 (LOC728658), mRNA. (A) | |
| 452 | ubiquinol-cytochrome c reductase hinge protein-like | UQCRHL |
| | (UQCRHL), mRNA. (A) | |
| 453 | lamin B receptor (LBR), transcript variant 1, mRNA. (I) | LBR |
| 454 | Ras and Rab interactor 2 (RIN2), mRNA. (S) | RIN2 |
| 455 | lactate dehydrogenase B (LDHB), mRNA. (S) | LDHB |
| 456 | granulysin (GNLY), transcript variant 519, mRNA. (A) | GNLY |
| 457 | ribosomal protein L12 (RPL12), mRNA. (S) | RPL12 |
| 458 | fer-1-like 3, myoferlin (C. elegans) (FER1L3), transcript | FER1L3 |
| | variant 2, mRNA. (A) | |
| 459 | nucleosome assembly protein 1-like 1 (NAP1L1), transcript | NAP1L1 |
| | variant 1, mRNA. (A) | |
| 460 | ribosomal protein L27 (RPL27), mRNA. (S) | RPL27 |
| 461 | RAB43, member RAS oncogene family (RAB43), mRNA. | RAB43 |
| | (S) | |
| 462 | hCG18290 (LOC644907), mRNA. (S) | LOC644907 |
| 463 | NADH dehydrogenase (ubiquinone) Fe—S protein 4, 18 kDa | NDUFS4 |
| | (NADH-coenzyme Q reductase) (NDUFS4), mRNA. (S) | |
| 464 | pleckstrin homology domain containing, family G (with | PLEKHG2 |
| | RhoGef domain) member 2 (PLEKHG2), mRNA. (S) | |
| 465 | RNA pseudouridylate synthase domain containing 4 | RPUSD4 |
| | (RPUSD4), mRNA. (S) | |
| 466 | H1 histone family, member X (H1FX), mRNA. (S) | H1FX |
| 467 | ribosomal protein L7a (RPL7A), mRNA. (S) | RPL7A |
| 468 | cytoskeleton associated protein 5 (CKAP5), transcript | CKAP5 |
| | variant 1, mRNA. (A) | |
| 469 | PREDICTED: hypothetical protein LOC731985 | LOC731985 |
| | (LOC731985), mRNA. (S) | |
| 470 | ribosomal protein L9 (RPL9), transcript variant 2, mRNA. | RPL9 |
| | (A) | |
| 471 | PREDICTED: misc_RNA (LOC100131609), miscRNA. | LOC100131609 |
| | (M) | |
| 472 | uridine-cytidine kinase 1-like 1 (UCKL1), mRNA. (S) | UCKL1 |
| 473 | MAX interactor 1 (MXI1), transcript variant 2, mRNA. (A) | MXI1 |
| 474 | fumarate hydratase (FH), nuclear gene encoding | FH |
| | mitochondrial protein, mRNA. (S) | |
| 475 | ST3 beta-galactoside alpha-2,3-sialyltransferase 5 | ST3GAL5 |
| | (ST3GAL5), transcript variant 2, mRNA. (S) | |
| 476 | ubiquitin specific peptidase 36 (USP36), mRNA. (S) | USP36 |
| 477 | HD domain containing 2 (HDDC2), mRNA. (S) | HDDC2 |
| 478 | acrosin binding protein (ACRBP), mRNA. (S) | ACRBP |
| 479 | eukaryotic translation initiation factor 3, subunit L (EIF3L), | EIF3L |
| | mRNA. (S) | |
| 480 | PREDICTED: similar to insulinoma protein (rig) | LOC729789 |
| | (LOC729789), mRNA. (A) | |
| 481 | solute carrier family 25, member 39 (SLC25A39), mRNA. | SLC25A39 |
| | (S) | |
| 482 | ras homolog gene family, member C (RHOC), transcript | RHOC |
| | variant 1, mRNA. (A) | |
| 483 | dolichyl-diphosphooligosaccharide--protein | LOC10012- |
| | | 8731 |
| | glycosyltransferase subunit 4 (LOC100128731), mRNA. (S) | |
| 484 | caspase 8, apoptosis-related cysteine peptidase (CASP8), | CASP8 |
| | transcript variant G, mRNA. (A) | |
| 485 | PREDICTED: misc_RNA (LOC100129553), miscRNA. | LOC10012- |
| | (M) | 9553 |
| 486 | PREDICTED: misc_RNA (LOC100131609), miscRNA. (A) | LOC100131609 |
| 487 | PREDICTED: similar to ribosomal protein L4 | LOC158345 |
| | (LOC158345), mRNA. (A) | |
| 488 | dynein, cytoplasmic 1, intermediate chain 2 (DYNC1I2), | DYNC1I2 |
| | mRNA. (S) | |
| 489 | Y box binding protein 1 (YBX1), mRNA. (S) | YBX1 |
| 490 | PREDICTED: misc_RNA (LOC728031), miscRNA. (M) | LOC728031 |
| 491 | prion protein (PRNP), transcript variant 2, mRNA. (A) | PRNP |
| 492 | chemokine (C—X—C motif) ligand 6 (granulocyte chemotactic | CXCL6 |
| | protein 2) (CXCL6), mRNA. (S) | |
| 493 | KTEL (Lys-Tyr-Glu-Leu) containing 1 (KTELC1), mRNA. | KTELC1 |
| | (A) | |
| 494 | heterogeneous nuclear ribonucleoprotein A1 pseudogene | LOC728643 |
| | (LOC728643), non-coding RNA. (S) | |
| 495 | reticulon 3 (RTN3), transcript variant 2, mRNA. (I) | RTN3 |
| 496 | NADH dehydrogenase (ubiquinone) Fe—S protein 5, 15 kDa | NDUFS5 |
| | (NADH-coenzyme Q reductase) (NDUFS5), mRNA. (S) | |
| 497 | PREDICTED: misc_RNA (LOC441013), miscRNA. (A) | LOC441013 |
| 498 | ribosomal protein S24 (RPS24), transcript variant 1, mRNA. | RPS24 |
| | (A) | |
| 499 | PREDICTED: similar to 40S ribosomal protein S16, | LOC441876 |
| | transcript variant 2 (LOC441876), mRNA. (A) | |
| 500 | PREDICTED: misc_RNA (LOC100132488), miscRNA. | LOC10013- |
| | (M) | 2488 |
| 501 | ribosomal protein L14 (RPL14), transcript variant 1, mRNA. | RPL14 |
| | (A) | |
| 502 | chaperonin containing TCP1, subunit 8 (theta) (CCT8), | CCT8 |
| | mRNA. (S) | |
| 503 | nuclear casein kinase and cyclin-dependent kinase substrate | NUCKS1 |
| | 1 (NUCKS1), mRNA. (S) | |
| 504 | copine VIII (CPNE8), mRNA. (S) | CPNE8 |
| 505 | PREDICTED: misc_RNA (LOC728693), miscRNA. (A) | LOC728693 |
| 506 | tetratricopeptide repeat domain 3 (TTC3), transcript variant | TTC3 |
| | 1, mRNA. (A) | |
| 507 | arginine vasopressin-induced 1 (AVPI1), mRNA. (S) | AVPI1 |
| 508 | protein tyrosine phosphatase, mitochondrial 1 (PTPMT1), | PTPMT1 |
| | nuclear gene encoding mitochondrial protein, mRNA. (S) | |
| 509 | PREDICTED: similar to peptidylprolyl isomerase A isoform | LOC341457 |
| | 1 (LOC341457), mRNA. (A) | |
| 510 | PREDICTED: similar to 60S ribosomal protein L29 (Cell | LOC643433 |
| | surface heparin binding protein HIP), transcript variant 1 | |
| | (LOC643433), mRNA. (A) | |
| 511 | oligosaccharyltransferase complex subunit (OSTC), mRNA. | OSTC |
| | (S) | |
| 512 | ribosomal protein L23 (RPL23), mRNA. (S) | RPL23 |
| 513 | ribosomal L1 domain containing 1 (RSL1D1), mRNA. (S) | RSL1D1 |
| 514 | ribosomal protein S6 (RPS6), mRNA. (S) | RPS6 |
| 515 | PREDICTED: misc_RNA (LOC387825), miscRNA. (I) | LOC387825 |
| 516 | PREDICTED: similar to ribosomal protein L9 | LOC651436 |
| | (LOC651436), mRNA. (S) | |
| 517 | PQ loop repeat containing 3 (PQLC3), mRNA. (S) | PQLC3 |
| 518 | methyltransferase like 7A (METTL7A), mRNA. (S) | METTL7A |
| 519 | PREDICTED: similar to 60S ribosomal protein L7a | LOC441034 |
| | (LOC441034), mRNA. (A) | |
| 520 | ribosomal protein L7 (RPL7), mRNA. (S) | RPL7 |
| 521 | PREDICTED: misc_RNA (RPS6P1), miscRNA. (A) | RPS6P1 |
| 522 | related RAS viral (r-ras) oncogene homolog (RRAS), | RRAS |
| | mRNA. (S) | |
| 523 | PREDICTED: misc_RNA (LOC729903), miscRNA. (A) | LOC729903 |
| 524 | PREDICTED: similar to ribosomal protein L21 | LOC728782 |
| | (LOC728782), mRNA. (A) | |
| 525 | nucleosome assembly protein 1-like 1 (NAP1L1), transcript | NAP1L1 |
| | variant 1, mRNA. (I) | |
| 526 | PREDICTED: similar to ribosomal protein L13a, transcript | LOC387841 |
| | variant 1 (LOC387841), mRNA. (A) | |
| 527 | PREDICTED: misc_RNA (LOC100132528), miscRNA. (I) | LOC100132528 |
| 528 | enoyl Coenzyme A hydratase 1, peroxisomal (ECH1), | ECH1 |
| | mRNA. (S) | |
| 529 | PREDICTED: hypothetical LOC400963 (LOC400963), | LOC400963 |
| | mRNA. (A) | |
| 530 | PREDICTED: similar to 40S ribosomal protein S15 (RIG | LOC440733 |
| | protein) (LOC440733), mRNA. (S) | |
| 531 | PREDICTED: similar to 60S ribosomal protein L7a, | LOC388474 |
| | transcript variant 3 (LOC388474), mRNA. (A) | |
| 532 | TBC1 domain family, member 9 (with GRAM domain) | TBC1D9 |
| | (TBC1D9), mRNA. (S) | |
| 533 | zu67a08.s1 Soares_testis_NHT cDNA clone | NaN |
| | IMAGE: 743030 3, mRNA sequence (S) | |
| 534 | dual specificity phosphatase 6 (DUSP6), transcript variant 1, | DUSP6 |
| | mRNA. (I) | |
| 535 | cell division cycle 25 homolog B (S. pombe) (CDC25B), | CDC25B |
| | transcript variant 2, mRNA. (A) | |
| 536 | chromosome 6 open reading frame 48 (C6orf48), transcript | C6orf48 |
| | variant 1, mRNA. (A) | |
| 537 | coronin, actin binding protein, 1A pseudogene | LOC606724 |
| | (LOC606724), non-coding RNA. (S) | |
| 538 | peptidylprolyl isomerase A (cyclophilin A)-like 4A | PPIAL4A |
| | (PPIAL4A), mRNA. (S) | |
| 539 | eukaryotic translation initiation factor 3, subunit L (EIF3L), | EIF3L |
| | mRNA. (S) | |
| 540 | protein phosphatase 1B (formerly 2C), magnesium- | PPM1B |
| | dependent, beta isoform (PPM1B), transcript variant 4, | |
| | mRNA. (I) | |
| 541 | acyl-Coenzyme A dehydrogenase, C-4 to C-12 straight | ACADM |
| | chain (ACADM), nuclear gene encoding mitochondrial | |
| | protein, mRNA. (S) | |
| 542 | PREDICTED: similar to mCG7602 (LOC100129902), | LOC10012- |
| | mRNA. (M) | 9902 |
| 543 | pseudouridylate synthase 1 (PUS1), transcript variant 2, | PUS1 |
| | mRNA. (A) | |
| 544 | NADH dehydrogenase (ubiquinone) 1 alpha subcomplex, 12 | NDUFA12 |
| | (NDUFA12), mRNA. (S) | |
| 545 | PREDICTED: similar to heterogeneous nuclear | LOC645691 |
| | ribonucleoprotein A1 (LOC645691), mRNA. (A) | |
| 546 | coiled-coil domain containing 6 (CCDC6), mRNA. (S) | CCDC6 |
| 547 | sushi domain containing 1 (SUSD1), mRNA. (S) | SUSD1 |
| 548 | host cell factor C1 (VP16-accessory protein) (HCFC1), | HCFC1 |
| | mRNA. (S) | |
| 549 | heat shock protein 90 kDa alpha (cytosolic), class B member | HSP90AB1 |
| | 1 (HSP90AB1), mRNA. (S) | |
| 550 | perilipin 2 (PLIN2), mRNA. (S) | PLIN2 |
| 551 | PREDICTED: similar to RAPGEF2 protein | LOC10013- |
| | (LOC100133567), partial mRNA. (S) | 3567 |
| 552 | eukaryotic translation elongation factor 2 (EEF2), mRNA. | EEF2 |
| | (S) | |
| 553 | PREDICTED: hypothetical protein LOC100130311 | LOC10013- |
| | (LOC100130311), mRNA. (M) | 0311 |
| 554 | ribosomal protein L11 (RPL11), mRNA. (S) | RPL11 |
| 555 | SIDI transmembrane family, member 2 (SIDT2), mRNA. | SIDT2 |
| | (S) | |
| 556 | ATP synthase, H+ transporting, mitochondrial F1 complex, | ATP5A1 |
| | alpha subunit 1, cardiac muscle (ATP5A1), nuclear gene | |
| | encoding mitochondrial protein, transcript variant 2, mRNA. | |
| | (A) | |
| 557 | PREDICTED: misc_RNA (LOC100132795), miscRNA. (A) | LOC10013- |
| | | 2795 |
| 558 | heat shock 27 kDa protein 1 (HSPB1), mRNA. (S) | HSPB1 |
| 559 | glutathione S-transferase pi (GSTP1), mRNA. (S) | GSTP1 |
| 560 | PREDICTED: similar to 40S ribosomal protein S29 | LOC647361 |
| | (LOC647361), mRNA. (S) | |
| 561 | PREDICTED: hypothetical gene supported by BC047417, | LOC400027 |
| | transcript variant 2 (LOC400027), mRNA. (A) | |
| 562 | G protein-coupled bile acid receptor 1 (GPBAR1), transcript | GPBAR1 |
| | variant 1, mRNA. (A) | |
| 563 | hydroxyacyl-Coenzyme A dehydrogenase (HADH), nuclear | HADH |
| | gene encoding mitochondrial protein, mRNA. (S) | |
| 564 | PREDICTED: misc_RNA (LOC100133273), miscRNA. | LOC10013- |
| | (M) | 3273 |
| 565 | PREDICTED: hypothetical LOC440575 (LOC440575), | LOC440575 |
| | mRNA. (M) | |
| 566 | PREDICTED: misc_RNA (LOC100129742), miscRNA. | LOC10012- |
| | (M) | 9742 |
| 567 | zinc finger, MYND domain containing 11 (ZMYND11), | ZMYND11 |
| | transcript variant 1, mRNA. (I) | |
| 568 | ribosomal protein S14 (RPS14), transcript variant 1, mRNA. | RPS14 |
| | (A) | |
| 569 | PREDICTED: similar to nuclease sensitive element binding | LOC646531 |
| | protein 1 (LOC646531), mRNA. (S) | |
| 570 | C-type lectin domain family 1, member B (CLEC1B), | CLEC1B |
| | transcript variant 1, mRNA. (S) | |
| 571 | chromosome 7 open reading frame 50 (C7orf50), mRNA. | C7orf50 |
| | (S) | |
| 572 | PREDICTED: similar to 60S acidic ribosomal protein P1 | LOC10012- |
| | (LOC100129028), mRNA. (M) | 9028 |
| 573 | PREDICTED: similar to 60S ribosomal protein L14 (CAG- | LOC649821 |
| | ISL 7), transcript variant 1 (LOC649821), mRNA. (A) | |
| 574 | chromosome 16 open reading frame 58 (C16orf58), mRNA. | C16orf58 |
| | (S) | |
| 575 | protein phosphatase 1, regulatory (inhibitor) subunit 10 | PPP1R10 |
| | (PPP1R10), mRNA. (S) | |
| 576 | PREDICTED: misc_RNA (LOC729679), miscRNA. (S) | LOC729679 |
| 577 | N-acetyltransferase 8B (GCN5-related, putative, | NAT8B |
| | gene/pseudogene) (NAT8B), mRNA. (S) | |
| 578 | PREDICTED: misc_RNA (LOC642828), miscRNA. (M) | LOC642828 |
| 579 | serine hydroxymethyltransferase 2 (mitochondrial) | SHMT2 |
| | (SHMT2), nuclear gene encoding mitochondrial protein, | |
| | mRNA. (S) | |
| 580 | DEAD (Asp-Glu-Ala-Asp) box polypeptide 17 (DDX17), | DDX17 |
| | transcript variant 1, mRNA. (I) | |
| 581 | small nucleolar RNA, H/ACA box 12 (SNORA12), small | SNORA12 |
| | nucleolar RNA. (S) | |
| 582 | non-metastatic cells 1, protein (NM23A) expressed in | NME1 |
| | (NME1), transcript variant 2, mRNA. (A) | |
| 583 | BEN domain containing 7 (BEND7), transcript variant 2, | BEND7 |
| | mRNA. (I) | |
| 584 | coiled-coil domain containing 14 (CCDC14), mRNA. (S) | CCDC14 |
| 585 | glyoxalase domain containing 4 (GLOD4), mRNA. (S) | GLOD4 |
| 586 | transferrin receptor (p90, CD71) (TFRC), mRNA. (S) | TFRC |
| 587 | PREDICTED: similar to ribosomal protein S2, transcript | LOC440589 |
| | variant 3 (LOC440589), mRNA. (S) | |
| 588 | PREDICTED: zinc finger protein 516 (ZNF516), mRNA. | ZNF516 |
| | (A) | |
| 589 | sushi domain containing 3 (SUSD3), mRNA. (S) | SUSD3 |
| 590 | ferritin, heavy polypeptide 1 (FTH1), mRNA. (S) | FTH1 |
| 591 | ribosomal protein S14 (RPS14), transcript variant 2, mRNA. | RPS14 |
| | (A) | |
| 592 | V-set and transmembrane domain containing 1 (VSTM1), | VSTM1 |
| | mRNA. (S) | |
| 593 | ribosomal protein, large, P0 (RPLP0), transcript variant 1, | RPLP0 |
| | mRNA. (A) | |
| 594 | jumonji domain containing 8 (JMJD8), mRNA. (S) | JMJD8 |
| 595 | PREDICTED: similar to similar to RPL23AP7 protein | LOC728481 |
| | (LOC728481), mRNA. (S) | |
| 596 | RAD21 homolog (S. pombe) (RAD21), mRNA. (S) | RAD21 |
| 597 | tweety homolog 3 (Drosophila) (TTYH3), mRNA. (S) | TTYH3 |
| 598 | chimerin (chimaerin) 2 (CHN2), transcript variant 2, | CHN2 |
| | mRNA. (A) | |
| 599 | aldehyde dehydrogenase 1 family, member A1 (ALDH1A1), | ALDH1A1 |
| | mRNA. (S) | |
| 600 | mitochondrial ribosomal protein L37 (MRPL37), nuclear | MRPL37 |
| | gene encoding mitochondrial protein, mRNA. (S) | |
| 601 | Josephin domain containing 1 (JOSD1), mRNA. (S) | JOSD1 |
| 602 | PREDICTED: similar to hCG1812832 (LOC729742), | LOC729742 |
| | mRNA. (A) | |
| 603 | translocase of outer mitochondrial membrane 40 homolog | TOMM40 |
| | (yeast) (TOMM40), nuclear gene encoding mitochondrial | |
| | protein, mRNA. (S) | |
| 604 | ATP synthase, H+ transporting, mitochondrial F1 complex, | ATP5A1 |
| | alpha subunit 1, cardiac muscle (ATP5A1), nuclear gene | |
| | encoding mitochondrial protein, transcript variant 2, mRNA. | |
| | (A) | |
| 605 | ornithine decarboxylase 1 (ODC1), mRNA. (S) | ODC1 |
| 606 | IMP4, U3 small nucleolar ribonucleoprotein, homolog | IMP4 |
| | (yeast) (IMP4), mRNA. (S) | |
| 607 | linker for activation of T cells (LAT), transcript variant 2, | LAT |
| | mRNA. (A) | |
| 608 | PREDICTED: hypothetical protein LOC100134504 | LOC10013- |
| | (LOC100134504), mRNA. (S) | 4504 |
| 609 | caspase 4, apoptosis-related cysteine peptidase (CASP4), | CASP4 |
| | transcript variant gamma, mRNA. (I) | |
| 610 | ribosomal protein S15a (RPS15A), transcript variant 2, | RPS15A |
| | mRNA. (A) | |
| 611 | integrin, beta 3 (platelet glycoprotein IIIa, antigen CD61) | ITGB3 |
| | (ITGB3), mRNA. (S) | |
| 612 | NLR family, pyrin domain containing 12 (NLRP12), | NLRP12 |
| | transcript variant 1, mRNA. (I) | |
| 613 | small nucleolar RNA, H/ACA box 23 (SNORA23), small | SNORA23 |
| | nucleolar RNA. (S) | |
| 614 | translocase of outer mitochondrial membrane 7 homolog | TOMM7 |
| | (yeast) (TOMM7), nuclear gene encoding mitochondrial | |
| | protein, mRNA. (S) | |
| 615 | granzyme B (granzyme 2, cytotoxic T-lymphocyte- | GZMB |
| | associated serine esterase 1) (GZMB), mRNA. (S) | |
| 616 | LAG1 homolog, ceramide synthase 6 (LASS6), mRNA. (S) | LASS6 |
| 617 | WD repeat domain 61 (WDR61), mRNA. (S) | WDR61 |
| 618 | integrin, beta 1 (fibronectin receptor, beta polypeptide, | ITGB1 |
| | antigen CD29 includes MDF2, MSK12) (ITGB1), transcript | |
| | variant 1C-2, mRNA. (A) | |
| 619 | barrier to autointegration factor 1 (BANF1), mRNA. (S) | BANF1 |
| 620 | ribosomal protein S27 (metallopanstimulin 1) (RPS27), | RPS27 |
| | mRNA. (S) | |
| 621 | SH3 domain and tetratricopeptide repeats 1 (SH3TC1), | SH3TC1 |
| | mRNA. (S) | |
| 622 | presenilin 1 (Alzheimer disease 3) (PSEN1), transcript | PSEN1 |
| | variant I-463, mRNA. (I) | |
| 623 | ankyrin repeat domain 9 (ANKRD9), mRNA. (S) | ANKRD9 |
| 624 | PREDICTED: similar to ribosomal protein L31 | LOC653773 |
| | (LOC653773), mRNA. (S) | |
| 625 | UDP-glucose ceramide glucosyltransferase-like 1 | UGCGL1 |
| | (UGCGL1), transcript variant 1, mRNA. (A) | |
| 626 | solute carrier family 30 (zinc transporter), member 1 | SLC30A1 |
| | (SLC30A1), mRNA. (S) | |
| 627 | BTB and CNC homology 1, basic leucine zipper | BACH1 |
| | transcription factor 1 (BACH1), transcript variant 3, mRNA. | |
| | (A) | |
| 628 | SDA1 domain containing 1 (SDAD1), mRNA. (S) | SDAD1 |
| 629 | thioredoxin-related transmembrane protein 4 (TMX4), | TMX4 |
| | mRNA. (S) | |
| 630 | PREDICTED: similar to interleukin 28B (LOC728942), | LOC728942 |
| | mRNA. (M) | |
| 631 | kelch-like 28 (Drosophila) (KLHL28), mRNA. (S) | KLHL28 |
| 632 | PREDICTED: hypothetical protein LOC100132742, | LOC10013- |
| | transcript variant 2 (LOC100132742), mRNA. (M) | 2742 |
| 633 | TSC22 domain family, member 3 (TSC22D3), transcript | TSC22D3 |
| | variant 2, mRNA. (A) | |
| 634 | acid phosphatase 1, soluble (ACP1), transcript variant 2, | ACP1 |
| | mRNA. (A) | |
| 635 | similar to ribosomal protein L19 (LOC653314), mRNA. (S) | LOC653314 |
| 636 | SMT3 suppressor of mif two 3 homolog 3 (S. cerevisiae) | SUMO3 |
| | (SUMO3), mRNA. (S) | |
| 637 | UBX domain protein 11 (UBXN11), transcript variant 2, | UBXN11 |
| | mRNA. (A) | |
| 638 | OAF homolog (Drosophila) (OAF), mRNA. (S) | OAF |
| 639 | copper metabolism (Murr1) domain containing 1 | COMMD1 |
| | (COMMD1), mRNA. (S) | |
| 640 | staphylococcal nuclease and tudor domain containing 1 | SND1 |
| | (SND1), mRNA. (S) | |
| 641 | inhibitor of Bruton agammaglobulinemia tyrosine kinase | IBTK |
| | (IBTK), mRNA. (S) | |
| 642 | PHD finger protein 13 (PHF13), mRNA. (S) | PHF13 |
| 643 | PREDICTED: misc_RNA (LOC728139), miscRNA. (A) | LOC728139 |
| 644 | extended synaptotagmin-like protein 1 (ESYT1), mRNA. | ESYT1 |
| | (S) | |
| 645 | PREDICTED: similar to 40S ribosomal protein S15 (RIG | LOC401019 |
| | protein), transcript variant 3 (LOC401019), mRNA. (A) | |
| 646 | eukaryotic translation initiation factor 3, subunit F (EIF3F), | EIF3F |
| | mRNA. (S) | |
| 647 | thymidylate synthetase (TYMS), mRNA. (S) | TYMS |
| 648 | junctional adhesion molecule 3 (JAM3), mRNA. (S) | JAM3 |
| 649 | ribosomal protein S16 (RPS16), mRNA. (S) | RPS16 |
| 650 | inner membrane protein, mitochondrial (mitofilin) (IMMT), | IMMT |
| | nuclear gene encoding mitochondrial protein, transcript | |
| | variant 2, mRNA. (S) | |
| 651 | peptidylprolyl isomerase (cyclophilin)-like 3 (PPIL3), | PPIL3 |
| | transcript variant PPIL3b, mRNA. (A) | |
| 652 | solute carrier family 2 (facilitated glucose transporter), | SLC2A6 |
| | member 6 (SLC2A6), mRNA. (S) | |
| 653 | spermidine/spermine N1-acetyltransferase family member 2 | SAT2 |
| | (SAT2), mRNA. (S) | |
| 654 | PREDICTED: hypothetical protein LOC100133923 | LOC10013- |
| | (LOC100133923), mRNA. (S) | 3923 |
| 655 | PREDICTED: misc_RNA (LOC388707), miscRNA. (A) | LOC388707 |
| 656 | zinc finger protein 593 (ZNF593), mRNA. (S) | ZNF593 |
| 657 | neuroblastoma breakpoint family, member 11 (NBPF11), | NBPF11 |
| | mRNA. (S) | |
| 658 | gasdermin B (GSDMB), transcript variant 2, mRNA. (I) | GSDMB |
| 659 | coiled-coil-helix-coiled-coil-helix domain containing 10 | CHCHD10 |
| | (CHCHD10), mRNA. (S) | |
| 660 | small Cajal body-specific RNA 3 (SCARNA3), guide RNA. | SCARNA3 |
| | (S) | |
| 661 | chemokine (C—X—C motif) ligand 6 (granulocyte chemotactic | CXCL6 |
| | protein 2) (CXCL6), mRNA. (S) | |
| 662 | glutamyl-prolyl-tRNA synthetase (EPRS), mRNA. (S) | EPRS |
| 663 | phytanoyl-CoA 2-hydroxylase (PHYH), transcript variant 2, | PHYH |
| | mRNA. (S) | |
| 664 | PREDICTED: misc_RNA (LOC100129379), miscRNA. (A) | LOC10012- |
| | | 9379 |
| 665 | ribosomal protein S2 (RPS2), mRNA. (S) | RPS2 |
| 666 | nucleoporin 205 kDa (NUP205), mRNA. (S) | NUP205 |
| 667 | general transcription factor II, i, pseudogene 1 (GTF2IP1) | GTF2IP1 |
| | on chromosome 7. (S) | |
| 668 | protein tyrosine phosphatase, receptor type, C (PTPRC), | PTPRC |
| | transcript variant 2, mRNA. (A) | |
| 669 | PREDICTED: misc_RNA (LOC286444), miscRNA. (A) | LOC286444 |
| 670 | ribosomal protein S27 (metallopanstimulin 1) (RPS27), | RPS27 |
| | mRNA. (S) | |
| 671 | PREDICTED: hypothetical protein LOC100131205, | LOC10013- |
| | transcript variant 3 (LOC100131205), mRNA. (A) | 1205 |
| 672 | ribosomal protein L35a (RPL35A), mRNA. (S) | RPL35A |
| 673 | transforming growth factor, beta receptor II (70/80 kDa) | TGFBR2 |
| | (TGFBR2), transcript variant 1, mRNA. (I) | |
| 674 | PREDICTED: misc_RNA (LOC643863), miscRNA. (M) | LOC643863 |
| 675 | vascular endothelial growth factor A (VEGFA), transcript | VEGFA |
| | variant 2, mRNA. (A) | |
| 676 | B melanoma antigen family, member 3 (BAGE3), mRNA. | BAGE3 |
| | (S) | |
| 677 | dehydrogenase/reductase (SDR family) X-linked (DHRSX), | DHRSX |
| | mRNA. (S) | |
| 678 | chromosome 5 open reading frame 13 (C5orf13), mRNA. | C5orf13 |
| | (S) | |
| 679 | mitochondrial ribosomal protein L11 (MRPL11), nuclear | MRPL11 |
| | gene encoding mitochondrial protein, transcript variant 1, | |
| | mRNA. (A) | |
| 680 | PREDICTED: misc_RNA (LOC100129141), miscRNA. | LOC10012- |
| | (M) | 9141 |
| 681 | cDNA FLJ30923 fis, clone FEBRA2006491 (S) | NaN |
| 682 | PREDICTED: misc_RNA (LOC728428), miscRNA. (M) | LOC728428 |
| 683 | pyridoxamine 5′-phosphate oxidase (PNPO), mRNA. (S) | PNPO |
| 684 | kinesin family member C3 (KIFC3), mRNA. (S) | KIFC3 |
| 685 | dCMP deaminase (DCTD), transcript variant 2, mRNA. (A) | DCTD |
| 686 | PREDICTED: similar to metallopanstimulin | LOC10013- |
| | (LOC100133812), mRNA. (S) | 3812 |
| 687 | potassium inwardly-rectifying channel, subfamily J, member | KCNJ15 |
| | 15 (KCNJ15), transcript variant 1, mRNA. (I) | |
| 688 | alveolar soft part sarcoma chromosome region, candidate 1 | ASPSCR1 |
| | (ASPSCR1), mRNA. (I) | |
| 689 | ribosomal protein L24 (RPL24), mRNA. (S) | RPL24 |
| 690 | PREDICTED: similar to 60S ribosomal protein L6 (TAX- | LOC646483 |
| | responsive enhancer element binding protein 107) | |
| | (TAXREB107) (Neoplasm-related protein C140), transcript | |
| | variant 1 (LOC646483), mRNA. (A) | |
| 691 | L antigen family, member 3 (LAGE3), mRNA. (S) | LAGE3 |
| 692 | 602508802F1 NIH_MGC_79 cDNA clone | NaN |
| | IMAGE: 4619448 5, mRNA sequence (S) | |
| 693 | chromosome 16 open reading frame 35 (C16orf35), | C16orf35 |
| | transcript variant 2, mRNA. (A) | |
| 694 | PREDICTED: similar to ribosomal protein L21 | LOC388532 |
| | (LOC388532), mRNA. (S) | |
| 695 | dipeptidyl-peptidase 7 (DPP7), mRNA. (I) | DPP7 |
| 696 | eukaryotic translation initiation factor 3, subunit B (EIF3B), | EIF3B |
| | transcript variant 1, mRNA. (S) | |
| 697 | lamin B receptor (LBR), transcript variant 1, mRNA. (A) | LBR |
| 698 | CD300c molecule (CD300C), mRNA. (S) | CD300C |
| 699 | PREDICTED: hypothetical LOC729402 (LOC729402), | LOC729402 |
| | mRNA. (A) | |
| 700 | TSC22 domain family, member 1 (TSC22D1), transcript | TSC22D1 |
| | variant 1, mRNA. (A) | |
| 701 | PREDICTED: similar to 60S acidic ribosomal protein P1, | LOC440927 |
| | transcript variant 4 (LOC440927), mRNA. (A) | |
| 702 | PREDICTED: similar to ribosomal protein L21 | LOC388621 |
| | (LOC388621), mRNA. (S) | |
| 703 | hCG1783417 (LOC401019), mRNA. (S) | LOC401019 |
| 704 | sorting and assembly machinery component 50 homolog (S. cerevisiae) | SAMM50 |
| | (SAMM50), mRNA. (S) | |
| 705 | D-2-hydroxyglutarate dehydrogenase (D2HGDH), nuclear | D2HGDH |
| | gene encoding mitochondrial protein, mRNA. (S) | |
| 706 | amine oxidase (flavin containing) domain 2 (AOF2), | AOF2 |
| | transcript variant 2, mRNA. (S) | |
| 707 | coiled-coil domain containing 90A (CCDC90A), mRNA. | CCDC90A |
| | (A) | |
| 708 | hypothetical protein LOC283392 (LOC283392), mRNA. (S) | LOC283392 |
| 709 | PREDICTED: misc_RNA (LOC100130980), miscRNA. (A) | LOC1001- |
| | | 30980 |
| 710 | tetraspanin 4 (TSPAN4), transcript variant 3, mRNA. (A) | TSPAN4 |
| 711 | diaphanous homolog 2 (Drosophila) (DIAPH2), transcript | DIAPH2 |
| | variant 156, mRNA. (S) | |
| 712 | zinc finger protein 22 (KOX 15) (ZNF22), mRNA. (S) | ZNF22 |
| 713 | PREDICTED: similar to 40S ribosomal protein S29 | LOC643284 |
| | (LOC643284), mRNA. (S) | |
| 714 | calsyntenin 1 (CLSTN1), transcript variant 1, mRNA. (A) | CLSTN1 |
| 715 | Sjogren syndrome antigen B (autoantigen La) (SSB), | SSB |
| | mRNA. (S) | |
| 716 | PREDICTED: similar to Ubiquinol-cytochrome c reductase | LOC729769 |
| | hinge protein (LOC729769), mRNA. (M) | |
| 717 | solute carrier family 30 (zinc transporter), member 1 | SLC30A1 |
| | (SLC30A1), mRNA. (S) | |
| 718 | PREDICTED: hypothetical protein LOC645232 | LOC645232 |
| | (LOC645232), mRNA. (S) | |
| 719 | phosphatidylinositol-specific phospholipase C, X domain | PLCXD1 |
| | containing 1 (PLCXD1), mRNA. (S) | |
| 720 | hCG1992539 (LOC91561), mRNA. (S) | LOC91561 |
| 721 | eukaryotic translation initiation factor 3, subunit M | EIF3M |
| | (EIF3M), mRNA. (S) | |
| 722 | myoferlin (MYOF), transcript variant 1, mRNA. (M) | MYOF |
| 723 | PREDICTED: chromosome 14 open reading frame 82 | C14orf82 |
| | (C14orf82), mRNA. (A) | |
| 724 | APEX nuclease (multifunctional DNA repair enzyme) 1 | APEX1 |
| | (APEX1), transcript variant 1, mRNA. (A) | |
| 725 | integrin, alpha 2b (platelet glycoprotein IIb of IIb/IIIa | ITGA2B |
| | complex, antigen CD41B) (ITGA2B), mRNA. (S) | |
| 726 | trafficking protein particle complex 6A (TRAPPC6A), | TRAPPC6A |
| | mRNA. (S) | |
| 727 | solute carrier family 25, member 43 (SLC25A43), mRNA. | SLC25A43 |
| | (S) | |
| 728 | tetraspanin 9 (TSPAN9), mRNA. (S) | TSPAN9 |
| 729 | BEN domain containing 7 (BEND7), transcript variant 1, | BEND7 |
| | mRNA. (A) | |
| 730 | PREDICTED: similar to 60S acidic ribosomal protein P2 | LOC643949 |
| | (LOC643949), mRNA. (S) | |
| 731 | PREDICTED: similar to ribosomal protein S14 | MGC87895 |
| | (MGC87895), mRNA. (A) | |
| 732 | non-protein coding RNA 219 (NCRNA00219), non-coding | NCRNA00- |
| | RNA. (S) | 219 |
| 733 | chemokine (C—X—C motif) receptor 5 (CXCR5), transcript | CXCR5 |
| | variant 2, mRNA. (A) | |
| 734 | microRNA let-7d (MIRLET7D), microRNA. (S) | MIRLET7D |
| 735 | basic transcription factor 3 (BTF3), transcript variant 1, | BTF3 |
| | mRNA. (A) | |
| 736 | epithelial membrane protein 1 (EMP1), mRNA. (S) | EMP1 |
| 737 | v-myb myeloblastosis viral oncogene homolog (avian) | MYB |
| | (MYB), transcript variant 2, mRNA. (S) | |
| 738 | ribonucleotide reductase M1 polypeptide (RRM1), mRNA. | RRM1 |
| | (S) | |
| 739 | ribose 5-phosphate isomerase A (RPIA), mRNA. (S) | RPIA |
| 740 | PREDICTED: hypothetical protein LOC100130892 | LOC10013- |
| | (LOC100130892), mRNA. (A) | 0892 |
| 741 | PREDICTED: hypothetical protein LOC648226 | LOC648226 |
| | (LOC648226), mRNA. (S) | |
| 742 | SEC11 homolog C (S. cerevisiae) (SEC11C), mRNA. (S) | SEC11C |
| 743 | G protein-coupled receptor 155 (GPR155), transcript variant | GPR155 |
| | 10, mRNA. (A) | |
| 744 | prolyl endopeptidase (PREP), mRNA. (S) | PREP |
| 745 | ribosome production factor 2 homolog (S. cerevisiae) | RPF2 |
| | (RPF2), mRNA. (S) | |
| 746 | chromosome 19 open reading frame 10 (C19orf10), mRNA. | C19orf10 |
| | (S) | |
| 747 | PREDICTED: hypothetical LOC653737 (LOC653737), | LOC653737 |
| | mRNA. (A) | |
| 748 | RAN binding protein 1 (RANBP1), mRNA. (S) | RANBP1 |
| 749 | pre T-cell antigen receptor alpha (PTCRA), mRNA. (S) | PTCRA |
| 750 | MOCO sulphurase C-terminal domain containing 1 | MOSC1 |
| | (MOSC1), mRNA. (S) | |
| 751 | minichromosome maintenance complex component 6 | MCM6 |
| | (MCM6), mRNA. (S) | |
| 752 | PREDICTED: misc_RNA (LOC100131940), miscRNA. | LOC10013- |
| | (M) | 1940 |
| 753 | chromosome 5 open reading frame 41 (C5orf41), mRNA. | C5orf41 |
| | (S) | |
| 754 | adducin 3 (gamma) (ADD3), transcript variant 1, mRNA. (I) | ADD3 |
| 755 | WD repeat domain 18 (WDR18), mRNA. (S) | WDR18 |
| 756 | cDNA FLJ38536 fis, clone HCHON2001200 (S) | NaN |
| 757 | cylindromatosis (turban tumor syndrome) (CYLD), mRNA. | CYLD |
| | (S) | |
| 758 | protein phosphatase 3 (formerly 2B), catalytic subunit, | PPP3CC |
| | gamma isoform (PPP3CC), mRNA. (S) | |
| 759 | coiled-coil domain containing 88C (CCDC88C), mRNA. (S) | CCDC88C |
| 760 | PREDICTED: misc_RNA (LOC729926), miscRNA. (M) | LOC729926 |
| 761 | unkempt homolog (Drosophila)-like (UNKL), transcript | UNKL |
| | variant 1, mRNA. (S) | |
| 762 | hyaluronoglucosaminidase 3 (HYAL3), mRNA. (S) | HYAL3 |
| 763 | chromosome 20 open reading frame 177 (C20orf177), | C20orf177 |
| | mRNA. (S) | |
| 764 | regulator of G-protein signaling 10 (RGS10), transcript | RGS10 |
| | variant 1, mRNA. (I) | |
| 765 | TAP binding protein (tapasin) (TAPBP), transcript variant 2, | TAPBP |
| | mRNA. (I) | |
| 766 | chromosome 10 open reading frame 46 (C10orf46), mRNA. | C10orf46 |
| | (S) | |
| 767 | PREDICTED: misc_RNA (LOC645157), miscRNA. (M) | LOC645157 |
| 768 | small nucleolar RNA, H/ACA box 1 (SNORA1), small | SNORA1 |
| | nucleolar RNA. (S) | |
| 769 | thrombomodulin (THBD), mRNA. (S) | THBD |
| 770 | PREDICTED: misc_RNA (LOC100132673), miscRNA. | LOC10013- |
| | (M) | 2673 |
| 771 | ST6 beta-galactosamide alpha-2,6-sialyltranferase 1 | ST6GAL1 |
| | (ST6GAL1), transcript variant 2, mRNA. (A) | |
| 772 | skeletal muscle and kidney enriched inositol phosphatase | SKIP |
| | (SKIP), transcript variant 2, mRNA. (I) | |
| 773 | CD99 molecule (CD99), transcript variant 1, mRNA. (S) | CD99 |
| 774 | ATP synthase, H+ transporting, mitochondrial F0 complex, | ATP5G2 |
| | subunit c (subunit 9), isoform 2 (ATP5G2), nuclear gene | |
| | encoding mitochondrial protein, transcript variant 2, mRNA. | |
| | (A) | |
| 775 | major histocompatibility complex, class II, DP beta 1 (HLA- | HLA-DPB1 |
| | DPB1), mRNA. (S) | |
| 776 | ribosomal protein L17-like (LOC645296), mRNA. (S) | LOC645296 |
| 777 | PREDICTED: misc_RNA (LOC646294), miscRNA. (M) | LOC646294 |
| 778 | unc-84 homolog A (C. elegans) (UNC84A), mRNA. (S) | UNC84A |
| 779 | proline rich Gla (G-carboxyglutamic acid) 4 | PRRG4 |
| | (transmembrane) (PRRG4), mRNA. (S) | |
| 780 | endoplasmic reticulum to nucleus signalling 1 (ERN1), | ERN1 |
| | transcript variant 2, mRNA. (I) | |
| 781 | uroporphyrinogen III synthase (congenital erythropoietic | UROS |
| | porphyria) (UROS), mRNA. (S) | |
| 782 | FYN oncogene related to SRC, FGR, YES (FYN), transcript | FYN |
| | variant 1, mRNA. (I) | |
| 783 | zinc finger protein 428 (ZNF428), mRNA. (S) | ZNF428 |
| 784 | PTK2 protein tyrosine kinase 2 (PTK2), transcript variant 2, | PTK2 |
| | mRNA. (A) | |
| 785 | PREDICTED: misc_RNA (LOC727865), miscRNA. (A) | LOC727865 |
| 786 | ATP-binding cassette, sub-family C (CFTR/MRP), member | ABCC5 |
| | 5 (ABCC5), transcript variant 2, mRNA. (I) | |
| 787 | PREDICTED: hypothetical protein LOC100133950 | LOC10013- |
| | (LOC100133950), mRNA. (S) | 3950 |
| 788 | general transcription factor II, i, pseudogene 1 (GTF2IP1) | GTF2IP1 |
| | on chromosome 7. (S) | |
| 789 | 4-aminobutyrate aminotransferase (ABAT), nuclear gene | ABAT |
| | encoding mitochondrial protein, transcript variant 2, mRNA. | |
| | (A) | |
| 790 | UDP-glucose ceramide glucosyltransferase-like 1 | UGCGL1 |
| | (UGCGL1), transcript variant 1, mRNA. (A) | |
| 791 | PREDICTED: hypothetical protein LOC100128126 | LOC10012- |
| | (LOC100128126), mRNA. (A) | 8126 |
| 792 | PREDICTED: misc_RNA (LOC643332), miscRNA. (A) | LOC643332 |
| 793 | clone 25194 mRNA sequence (S) | NaN |
| 794 | asialoglycoprotein receptor 2 (ASGR2), transcript variant 3, | ASGR2 |
| | mRNA. (A) | |
| 795 | PREDICTED: neuroblastoma breakpoint family, member 1, | NBPF1 |
| | transcript variant 16 (NBPF1), mRNA. (I) | |
| 796 | PREDICTED: misc_RNA (LOC728820), miscRNA. (A) | LOC728820 |
| 797 | guanylate cyclase 1, soluble, alpha 3 (GUCY1A3), mRNA. | GUCY1A3 |
| | (S) | |
| 798 | hCG39912 (LOC642250), mRNA. (A) | LOC642250 |
| 799 | protein tyrosine phosphatase, receptor type, C (PTPRC), | PTPRC |
| | transcript variant 4, mRNA. (A) | |
| 800 | splicing factor, arginine/serine-rich 2B (SFRS2B), mRNA. | SFRS2B |
| | (S) | |
| 801 | BMS1 homolog, ribosome assembly protein (yeast) | BMS1 |
| | (BMS1), mRNA. (S) | |
| 802 | coiled-coil domain containing 56 (CCDC56), mRNA. (S) | CCDC56 |
| 803 | v-myc myelocytomatosis viral oncogene homolog (avian) | MYC |
| | (MYC), mRNA. (S) | |
| 804 | CCAAT/enhancer binding protein (C/EBP), epsilon | CEBPE |
| | (CEBPE), mRNA. (S) | |
| 805 | cold shock domain containing E1, RNA-binding (CSDE1), | CSDE1 |
| | transcript variant 1, mRNA. (I) | |
| 806 | aldehyde dehydrogenase 1 family, member A1 (ALDH1A1), | ALDH1A1 |
| | mRNA. (S) | |
| 807 | PREDICTED: KIAA0194 protein (KIAA0194), mRNA. | KIAA0194 |
| | (M) | |
| 808 | myelin basic protein (MBP), transcript variant 3, mRNA. | MBP |
| | (A) | |
| 809 | small nucleolar RNA, H/ACA box 46 (SNORA46), small | SNORA46 |
| | nucleolar RNA. (S) | |
| 810 | aquaporin 10 (AQP10), mRNA. (S) | AQP10 |
| 811 | microRNA 744 (MIR744), microRNA. (S) | MIR744 |
| 812 | PREDICTED: misc_RNA (LOC727865), miscRNA. (M) | LOC727865 |
| 813 | PREDICTED: misc_RNA (LOC729208), miscRNA. (M) | LOC729208 |
| 814 | jumonji domain containing 8 (JMJD8), mRNA. (S) | JMJD8 |
| 815 | FLJ38717 protein (FLJ38717), mRNA. (S) | FLJ38717 |
| 816 | PREDICTED: misc_RNA (LOC645173), miscRNA. (A) | LOC645173 |
| 817 | PREDICTED: misc_RNA (L00730187), miscRNA. (M) | LOC730187 |
| 818 | PREDICTED: misc_RNA (LOC643358), miscRNA. (A) | LOC643358 |
| 819 | proteasome (prosome, macropain) assembly chaperone 1 | PSMG1 |
| | (PSMG1), transcript variant 2, mRNA. (A) | |
| 820 | E74-like factor 2 (ets domain transcription factor) (ELF2), | ELF2 |
| | transcript variant 1, mRNA. (I) | |
| 821 | TBC1 domain family, member 9B (with GRAM domain) | TBC1D9B |
| | (TBC1D9B), transcript variant 2, mRNA. (A) | |
| 822 | ilvB (bacterial acetolactate synthase)-like (ILVBL), mRNA. | ILVBL |
| | (A) | |
| 823 | zyg-11 homolog B (C. elegans) (ZYG11B), mRNA. (S) | ZYG11B |
| 824 | TAR DNA binding protein (TARDBP), mRNA. (S) | TARDBP |
| 825 | schlafen family member 11 (SLFN11), mRNA. (S) | SLFN11 |
| 826 | PREDICTED: hypothetical protein LOC100133931 | LOC10013- |
| | (LOC100133931), mRNA. (S) | 3931 |
| 827 | mitogen-activated protein kinase kinase kinase 4 | MAP3K4 |
| | (MAP3K4), transcript variant 1, mRNA. (A) | |
| 828 | CD86 antigen (CD28 antigen ligand 2, B7-2 antigen) | CD86 |
| | (CD86), transcript variant 1, mRNA. (I) | |
| 829 | RUN and SH3 domain containing 1 (RUSC1), mRNA. (S) | RUSC1 |
| 830 | PREDICTED: misc_RNA (LOC646819), miscRNA. (M) | LOC646819 |
| 831 | GPN-loop GTPase 1 (GPN1), mRNA. (S) | GPN1 |
| 832 | nucleobindin 2 (NUCB2), mRNA. (S) | NUCB2 |
| 833 | docking protein 2, 56 kDa (DOK2), mRNA. (A) | DOK2 |
| 834 | adducin 3 (gamma) (ADD3), transcript variant 3, mRNA. | ADD3 |
| | (A) | |
| 835 | mitochondrial ribosomal protein L45 (MRPL45), nuclear | MRPL45 |
| | gene encoding mitochondrial protein, mRNA. (A) | |
| 836 | block of proliferation 1 (BOP1), mRNA. (S) | BOP1 |
| 837 | phosducin-like 3 (PDCL3), mRNA. (S) | PDCL3 |
| 838 | PREDICTED: hypothetical LOC653232, transcript variant 4 | LOC653232 |
| | (LOC653232), mRNA. (A) | |
| 839 | transcription elongation factor A (SII)-like 4 (TCEAL4), | TCEAL4 |
| | transcript variant 4, mRNA. (A) | |
| 840 | G protein-coupled bile acid receptor 1 (GPBAR1), transcript | GPBAR1 |
| | variant 3, mRNA. (S) | |
| 841 | nuclear fragile X mental retardation protein interacting | NUFIP2 |
| | protein 2 (NUFIP2), mRNA. (S) | |
| 842 | immunoglobulin superfamily, member 6 (IGSF6), mRNA. | IGSF6 |
| | (S) | |
| 843 | collagen, type XVII, alpha 1 (COL17A1), mRNA. (I) | COL17A1 |
| 844 | AGENCOURT_14354957 NIH_MGC_191 cDNA clone | NaN |
| | IMAGE: 30413554 5, mRNA sequence (S) | |
| 845 | PREDICTED: misc_RNA (LOC648729), miscRNA. (S) | LOC648729 |
| 846 | AXIN1 up-regulated 1 (AXUD1), mRNA. (S) | AXUD1 |
| 847 | cDNA FLJ44370 fis, clone TRACH3008902 (S) | NaN |
| 848 | PREDICTED: similar to ubiquitin and ribosomal protein | LOC388720 |
| | S27a precursor (LOC388720), mRNA. (A) | |
| 849 | ligatin (LGTN), mRNA. (S) | LGTN |
| 850 | CD19 molecule (CD19), mRNA. (S) | CD19 |
| 851 | exportin 4 (XPO4), mRNA. (S) | XPO4 |
| 852 | PREDICTED: misc_RNA (LOC644563), miscRNA. (A) | LOC644563 |
| 853 | serpin peptidase inhibitor, clade B (ovalbumin), member 2 | SERPINB2 |
| | (SERPINB2), mRNA. (S) | |
| 854 | myelin protein zero-like 3 (MPZL3), mRNA. (S) | MPZL3 |
| 855 | RPA interacting protein (RPAIN), mRNA. (I) | RPAIN |
| 856 | chromosome 5 open reading frame 41 (C5orf41), mRNA. | C5orf41 |
| | (S) | |
| 857 | bestrophin 1 (BEST1), mRNA. (S) | BEST1 |
| 858 | GTPase, IMAP family member 7 (GIMAP7), mRNA. (S) | GIMAP7 |
| 859 | myeloproliferative leukemia virus oncogene (MPL), mRNA. | MPL |
| | (S) | |
| 860 | PREDICTED: hypothetical LOC644790 (LOC644790), | LOC644790 |
| | mRNA. (M) | |
| 861 | NOP2 nucleolar protein homolog (yeast) (NOP2), transcript | NOP2 |
| | variant 1, mRNA. (A) | |
| 862 | guanine nucleotide binding protein (G protein), beta | GNB4 |
| | polypeptide 4 (GNB4), mRNA. (S) | |
| 863 | LSM5 homolog, U6 small nuclear RNA associated (S. cerevisiae) | LSM5 |
| | (LSM5), mRNA. (S) | |
| 864 | guanine nucleotide binding protein (G protein), beta | GNB4 |
| | polypeptide 4 (GNB4), mRNA. (S) | |
| 865 | cytidine monophosphate (UMP-CMP) kinase 1, cytosolic | CMPK1 |
| | (CMPK1), mRNA. (S) | |
| 866 | PREDICTED: misc_RNA (LOC728532), miscRNA. (A) | LOC728532 |
| 867 | cytidine monophosphate N-acetylneuraminic acid synthetase | CMAS |
| | (CMAS), mRNA. (S) | |
| 868 | mitochondrial ribosomal protein S24 (MRPS24), nuclear | MRPS24 |
| | gene encoding mitochondrial protein, mRNA. (S) | |
| 869 | adenosine deaminase (ADA), mRNA. (S) | ADA |
| 870 | olfactory receptor, family 4, subfamily K, member 15 | OR4K15 |
| | (OR4K15), mRNA. (S) | |
| 871 | PREDICTED: similar to hCG2024106, transcript variant 2 | LOC10013- |
| | (LOC100134648), mRNA. (A) | 4648 |
| 872 | AT rich interactive domain 4B (RBP1-like) (ARID4B), | ARID4B |
| | transcript variant 1, mRNA. (A) | |
| 873 | PREDICTED: similar to lethal (2) k00619 CG4775-PA | LOC729148 |
| | (LOC729148), mRNA. (A) | |
| 874 | oligodendrocyte myelin glycoprotein (OMG), mRNA. (S) | OMG |
| 875 | PREDICTED: similar to 60S ribosomal protein L21, | LOC731640 |
| | transcript variant 2 (LOC731640), mRNA. (A) | |
| 876 | FNPARC07 FNP cDNA, mRNA sequence (S) | NaN |
| 877 | transmembrane protein 173 (TMEM173), nuclear gene | TMEM173 |
| | encoding mitochondrial protein, mRNA. (S) | |
| 878 | hypothetical protein LOC285074 (LOC285074), mRNA. (I) | LOC285074 |
| 879 | aldo-keto reductase family 7, member A2 (aflatoxin | AKR7A2 |
| | aldehyde reductase) (AKR7A2), mRNA. (S) | |
| 880 | ribosomal protein L31 (RPL31), transcript variant 1, mRNA. | RPL31 |
| | (S) | |
| 881 | secernin 1 (SCRN1), mRNA. (S) | SCRN1 |
| 882 | cDNA FLJ26692 fis, clone MPG07890 (S) | NaN |
| 883 | zinc finger protein 668 (ZNF668), mRNA. (S) | ZNF668 |
| 884 | similar to CG32542-PA (LOC196752), mRNA. (S) | LOC196752 |
| 885 | PREDICTED: similar to Brix domain containing 1 | LOC729608 |
| | (LOC729608), mRNA. (A) | |
| 886 | mRNA; cDNA DKFZp779M2422 (from clone | NaN |
| | DKFZp779M2422) (S) | |
| 887 | topoisomerase (DNA) III alpha (TOP3A), mRNA. (S) | TOP3A |
| 888 | nasal embryonic LHRH factor (NELF), mRNA. (S) | NELF |
| 889 | poly (ADP-ribose) polymerase family, member 1 (PARP1), | PARP1 |
| | mRNA. (S) | |
| 890 | ADP-ribosylation factor-like 2 (ARL2), mRNA. (S) | ARL2 |
| 891 | retinol dehydrogenase 14 (all-trans/9-cis/11-cis) (RDH14), | RDH14 |
| | mRNA. (S) | |
| 892 | RNA binding motif protein 33 (RBM33), transcript variant | RBM33 |
| | 1, mRNA. (S) | |
| 893 | PREDICTED: hypothetical LOC728590 (LOC728590), | LOC728590 |
| | mRNA. (M) | |
| 894 | enolase-phosphatase 1 (ENOPH1), mRNA. (S) | ENOPH1 |
| 895 | PREDICTED: p21 (CDKN1A)-activated kinase 2 (PAK2), | PAK2 |
| | mRNA. (A) | |
| 896 | chromosome 19 open reading frame 48 (C19orf48), mRNA. | C19orf48 |
| | (A) | |
| 897 | gem (nuclear organelle) associated protein 4 (GEMIN4), | GEMIN4 |
| | mRNA. (S) | |
| 898 | PREDICTED: similar to metallopanstimulin | LOC10013- |
| | (LOC100130070), mRNA. (M) | 0070 |
| 899 | acetyl-Coenzyme A acetyltransferase 1 (ACAT1), nuclear | ACAT1 |
| | gene encoding mitochondrial protein, mRNA. (S) | |
| 900 | PREDICTED: similar to cell division cycle 2-like 2 isoform | LOC647384 |
| | 3, transcript variant 1 (LOC647384), mRNA. (A) | |
| 901 | stabilin 1 (STAB1), mRNA. (S) | STAB1 |
| 902 | PREDICTED: similar to 60S ribosomal protein L36 | LOC127295 |
| | (LOC127295), mRNA. (S) | |
| 903 | PREDICTED: similar to ribosomal protein S27 | LOC648622 |
| | (LOC648622), mRNA. (S) | |
| 904 | PR domain containing 1, with ZNF domain (PRDM1), | PRDM1 |
| | transcript variant 2, mRNA. (I) | |
| 905 | DIM1 dimethyladenosine transferase 1-like (S. cerevisiae) | DIMT1L |
| | (DIMT1L), mRNA. (S) | |
| 906 | nucleoporin 210 kDa (NUP210), mRNA. (S) | NUP210 |
| 907 | PREDICTED: heterogeneous nuclear ribonucleoprotein A1 | HNRPA1P4 |
| | pseudogene 4 (HNRPA1P4), mRNA. (A) | |
| 908 | ribosomal protein L17 (RPL17), transcript variant 2, mRNA. | RPL17 |
| | (A) | |
| 909 | ureidopropionase, beta (UPB1), mRNA. (S) | UPB1 |
| 910 | tumor necrosis factor (ligand) superfamily, member 15 | TNFSF15 |
| | (TNFSF15), mRNA. (S) | |
| 911 | PREDICTED: similar to rCG23287 (LOC728590), mRNA. | LOC728590 |
| | (A) | |
| 912 | PREDICTED: misc_RNA (LOC100132493), miscRNA. (A) | LOC10013- |
| | | 2493 |
| 913 | PREDICTED: similar to CG33774-PA (LOC400948), | LOC400948 |
| | mRNA. (A) | |
| 914 | topoisomerase (DNA) II alpha 170 kDa (TOP2A), mRNA. | TOP2A |
| | (S) | |
| 915 | SAM and SH3 domain containing 1 (SASH1), mRNA. (S) | SASH1 |
| 916 | T-cell leukemia/lymphoma 1B (TCL1B), transcript variant | TCL1B |
| | 1, mRNA. (A) | |
| 917 | interleukin enhancer binding factor 3, 90 kDa (ILF3), | ILF3 |
| | transcript variant 1, mRNA. (I) | |
| 918 | chromosome 19 open reading frame 2 (C19orf2), transcript | C19orf2 |
| | variant 2, mRNA. (A) | |
| 919 | chromosome 1 open reading frame 183 (C1orf183), | C1orf183 |
| | transcript variant 2, mRNA. (A) | |
| 920 | RNA binding motif protein 12B (RBM12B), mRNA. (S) | RBM12B |
| 921 | CTAGE family, member 5 (CTAGE5), transcript variant 4, | CTAGE5 |
| | mRNA. (A) | |
| 922 | phosphodiesterase 5A, cGMP-specific (PDE5A), transcript | PDE5A |
| | variant 1, mRNA. (A) | |
| 923 | dual specificity phosphatase 6 (DUSP6), transcript variant 2, | DUSP6 |
| | mRNA. (A) | |
| 924 | myelin basic protein (MBP), transcript variant 7, mRNA. | MBP |
| | (A) | |
| 925 | leucine-rich repeat kinase 2 (LRRK2), mRNA. (S) | LRRK2 |
| 926 | glutaminyl-tRNA synthetase (QARS), mRNA. (S) | QARS |
| 927 | fatty acid synthase (FASN), mRNA. (S) | FASN |
| 928 | TH1-like (Drosophila) (TH1L), transcript variant 1, mRNA. | TH1L |
| | (I) | |
| 929 | adenylate cyclase 3 (ADCY3), mRNA. (S) | ADCY3 |
| 930 | ATP synthase, H+ transporting, mitochondrial F0 complex, | ATP5G1 |
| | subunit C1 (subunit 9) (ATP5G1), nuclear gene encoding | |
| | mitochondrial protein, transcript variant 2, mRNA. (A) | |
| 931 | TIGA1 (TIGA1), mRNA. (S) | TIGA1 |
| 932 | zinc finger protein 33A (ZNF33A), transcript variant 2, | ZNF33A |
| | mRNA. (S) | |
| 933 | chromosome 1 open reading frame 63 (C1orf63), transcript | C1orf63 |
| | variant 1, mRNA. (I) | |
| 934 | ribosomal protein S27a (RPS27A), mRNA. (S) | RPS27A |
| 935 | ubiquinol-cytochrome c reductase hinge protein (UQCRH), | UQCRH |
| | mRNA. (S) | |
| 936 | integrin, beta 7 (ITGB7), mRNA. (S) | ITGB7 |
| 937 | amino-terminal enhancer of split (AES), transcript variant 2, | AES |
| | mRNA. (A) | |
| 938 | ATP-binding cassette, sub-family B (MDR/TAP), member | ABCB10 |
| | 10 (ABCB10), nuclear gene encoding mitochondrial protein, | |
| | mRNA. (S) | |
| 939 | PREDICTED: similar to GMP synthase [glutamine- | LOC728564 |
| | hydrolyzing] (Glutamine amidotransferase) (GMP | |
| | synthetase) (LOC728564), mRNA. (S) | |
| 940 | HRAS-like suppressor 3 (HRASLS3), mRNA. (S) | HRASLS3 |
| 941 | ribosomal protein L17 (RPL17), transcript variant 2, mRNA. | RPL17 |
| | (S) | |
| 942 | PX domain containing serine/threonine kinase (PXK), | PXK |
| | mRNA. (S) | |
| 943 | killer cell lectin-like receptor subfamily B, member 1 | KLRB1 |
| | (KLRB1), mRNA. (S) | |
| 944 | RAB22A, member RAS oncogene family (RAB22A), | RAB22A |
| | mRNA. (S) | |
| 945 | PREDICTED: similar to septin 7, transcript variant 4 | LOC644162 |
| | (LOC644162), mRNA. (A) | |
| 946 | platelet-activating factor receptor (PTAFR), mRNA. (S) | PTAFR |
| 947 | ribosomal protein L13 (RPL13), transcript variant 2, mRNA. | RPL13 |
| | (A) | |
| 948 | small Cajal body-specific RNA 21 (SCARNA21), guide | SCARNA21 |
| | RNA. (S) | |
| 949 | bromodomain containing 8 (BRD8), transcript variant 3, | BRD8 |
| | mRNA. (A) | |
| 950 | HscB iron-sulfur cluster co-chaperone homolog (E. coli) | HSCB |
| | (HSCB), mRNA. (S) | |
| 951 | endoplasmic reticulum-golgi intermediate compartment | ERGIC1 |
| | (ERGIC) 1 (ERGIC1), transcript variant 1, mRNA. (I) | |
| 952 | chromosome 7 open reading frame 38 (C7orf38), mRNA. | C7orf38 |
| | (S) | |
| 953 | tetratricopeptide repeat domain 4 (TTC4), mRNA. (S) | TTC4 |
| 954 | PREDICTED: misc_RNA (LOC729301), miscRNA. (M) | LOC729301 |
| 955 | chromosome X open reading frame 26 (CXorf26), mRNA. | CXorf26 |
| | (S) | |
| 956 | family with sequence similarity 160, member B1 | FAM160B1 |
| | (FAM160B1), transcript variant 1, mRNA. (I) | |
| 957 | PREDICTED: misc_RNA (LOC148430), miscRNA. (M) | LOC148430 |
| 958 | CDC28 protein kinase regulatory subunit 1B (CKS1B), | CKS1B |
| | mRNA. (S) | |
| 959 | RWD domain containing 1 (RWDD1), transcript variant 3, | RWDD1 |
| | mRNA. (A) | |
| 960 | phosphatidylinositol glycan anchor biosynthesis, class P | PIGP |
| | (PIGP), transcript variant 2, mRNA. (A) | |
| 961 | ribosomal protein, large, P1 (RPLP1), transcript variant 1, | RPLP1 |
| | mRNA. (A) | |
| 962 | PREDICTED: similar to ribonucleic acid binding protein | LOC643446 |
| | S1, transcript variant 2 (LOC643446), mRNA. (A) | |
| 963 | eukaryotic translation initiation factor 3, subunit M | EIF3M |
| | (EIF3M), mRNA. (S) | |
| 964 | ubiquitin-conjugating enzyme E2B (RAD6 homolog) | UBE2B |
| | (UBE2B), mRNA. (S) | |
| 965 | NADH dehydrogenase (ubiquinone) 1 alpha subcomplex, 9, | NDUFA9 |
| | 39 kDa (NDUFA9), mRNA. (S) | |
| 966 | PREDICTED: similar to hCG1640454 (LOC391656), | LOC391656 |
| | mRNA. (S) | |
| 967 | calpain 3, (p94) (CAPN3), transcript variant 2, mRNA. (A) | CAPN3 |
| 968 | chromodomain helicase DNA binding protein 1-like | CHD1L |
| | (CHD1L), mRNA. (S) | |
| 969 | potassium inwardly-rectifying channel, subfamily J, member | KCNJ2 |
| | 2 (KCNJ2), mRNA. (S) | |
| 970 | PREDICTED: hypothetical protein LOC100131096 | LOC10013- |
| | (LOC100131096), mRNA. (M) | 1096 |
| 971 | PREDICTED: similar to general transcription factor II, i | LOC652771 |
| | isoform 1 (LOC652771), mRNA. (S) | |
| 972 | chromosome 12 open reading frame 57 (C12orf57), mRNA. | C12orf57 |
| | (S) | |
| 973 | hyaluronan synthase 1 (HAS1), mRNA. (S) | HAS1 |
| 974 | eukaryotic translation initiation factor 3, subunit G (EIF3G), | EIF3G |
| | mRNA. (S) | |
| 975 | dual-specificity tyrosine-(Y)-phosphorylation regulated | DYRK4 |
| | kinase 4 (DYRK4), mRNA. (S) | |
| 976 | family with sequence similarity 46, member A (FAM46A), | FAM46A |
| | mRNA. (S) | |
| 977 | PREDICTED: hypothetical LOC728518 (LOC728518), | LOC728518 |
| | mRNA. (A) | |
| 978 | PREDICTED: similar to ubiquitin specific protease 32, | LOC650546 |
| | transcript variant 1 (LOC650546), mRNA. (A) | |
| 979 | nicotinamide nucleotide transhydrogenase (NNT), nuclear | NNT |
| | gene encoding mitochondrial protein, transcript variant 1, | |
| | mRNA. (A) | |
| 980 | methyltransferase like 5 (METTL5), mRNA. (S) | METTL5 |
| 981 | ubiquitin specific peptidase 5 (isopeptidase T) (USP5), | USP5 |
| | transcript variant 2, mRNA. (S) | |
| 982 | SIL1 homolog, endoplasmic reticulum chaperone (S. cerevisiae) | SIL1 |
| | (SIL1), transcript variant 1, mRNA. (S) | |
| 983 | PREDICTED: similar to ALR-like protein (LOC645159), | LOC645159 |
| | mRNA. (S) | |
| 984 | regulatory factor X, 7 (RFX7), mRNA. (S) | RFX7 |
| 985 | melanoma antigen family D, 1 (MAGED1), transcript | MAGED1 |
| | variant 3, mRNA. (A) | |

By “isoform” or “multiple molecular form” is meant an alternative expression product or variant of a single gene in a given species, including forms generated by alternative splicing, single nucleotide polymorphisms, alternative promoter usage, alternative translation initiation small genetic differences between alleles of the same gene, and posttranslational modifications (PTMs) of these sequences.

By “related proteins” or “proteins of the same family” are meant expression products of different genes or related genes identified as belonging to a common family. Related proteins in the same biomarker family, e.g., LOX-1, may or may not share related functions. Related proteins can be readily identified as having significant sequence identity either over the entire protein or a significant part of the protein that is typically referred to as a “domain”. Proteins with at least 20% sequence homology or sequence identity can be readily identified as belonging to the same protein family.

By “homologous protein” is meant an alternative form of a related protein produced from a related gene having a percent sequence similarity or identity of greater than 20%, greater than 30%, greater than 40%, greater than 50%, greater than 60%, greater than 70%, greater than 75%, greater than 80%, greater than 85%, greater than 90%, greater than 95%, greater than 97%, or greater than 99%.

The term “ligand” with regard to protein biomarkers refers to a molecule that binds or complexes, with the PMN-MDSC biomarker protein, e.g., LOX-1. Thus, a ligand can be an amino acid sequence or protein sequence, or a molecular form or peptide, such as an antibody, antibody mimic or equivalent, or a fragment thereof. The ligand can be a naturally occurring peptide that binds to a portion of the LOX-1 receptor or a synthetically or recombinantly produced chimeric peptide having a portion that binds to the LOX-1 receptor and a portion designed for other purposes, e.g., to assist in the detection of the binding. Similarly, the peptide may be designed, or a small molecule designed, to bind to LOX-1 by mimicking the three-dimensional physical structure of the LOX-1 receptor. The term ligand as used with respect to the neutrophil biomarkers, e.g., CD15 and CD66b, and the PMN-MDSC signature biomarkers identified herein refers to similar amino acid sequences, peptides, chimeric proteins, etc, which can bind with the respective cell proteins.

The term “ligand” with regarding to a nucleic acid sequence encoding a biomarker, refers to a molecule that binds or complexes, with the indicated biomarker nucleic acid, e.g., LOX-1 DNA or RNA. Such a ligand can itself be an antibody or antibody fragment, a nucleotide sequence, e.g., a polynucleotide or oligonucleotide, primer or probe, which can be complementary to the biomarker-encoding sequence.

As used herein for the described methods and compositions, the term “antibody” refers to an intact immunoglobulin having two light and two heavy chains or fragments thereof capable of binding to a biomarker protein or a fragment of a biomarker protein. Thus a single isolated antibody or an antigen-binding fragment thereof may be a monoclonal antibody, a synthetic antibody, a recombinant antibody, a chimeric antibody, a humanized antibody, a human antibody, or a bi-specific antibody or multi-specific construct that can bind two or more target biomarkers.

The term “antibody fragment” as used herein for the described methods and compositions refers to less than an intact antibody structure having antigen-binding ability. Such fragments, include, without limitation, an isolated single antibody chain or an scFv fragment, which is a recombinant molecule in which the variable regions of light and heavy immunoglobulin chains encoding antigen-binding domains are engineered into a single polypeptide. Other scFV constructs include diabodies, i.e., paired scFvs or non-covalent dimers of scFvs that bind to one another through complementary regions to form bivalent molecules. Still other scFV constructs include complementary scFvs produced as a single chain (tandem scFvs) or bispecific tandem scFvs.

Other antibody fragments include an Fv construct, a Fab construct, an Fc construct, a light chain or heavy chain variable or complementarity determining region (CDR) sequence, etc. Still other antibody fragments include monovalent or bivalent minibodies (miniaturized monoclonal antibodies) which are monoclonal antibodies from which the domains non-essential to function have been removed. In one embodiment, a minibody is composed of a single-chain molecule containing one V_{L}, one V_{H} antigen-binding domain, and one or two constant “effector” domains. These elements are connected by linker domains. In still another embodiment, the antibody fragments useful in the methods and compositions herein are “unibodies”, which are IgG4 molecules from with the hinge region has been removed. See, reference 56 and the documents cited thereon for other forms of antibodies useful in these methods and compositions. For example, a LOX-1 antibody is available from commercial sources, such as Biolegend Inc., San Diego, Calif. Anti-LOX-1 antibodies or the antagonists or inhibitors referred to herein for the ER stress targets or other targeted biomarkers may also be any of these forms of antibody or antibody fragments.

As used herein, “labels” or “reporter molecules” or “detectable label components” are chemical or biochemical moieties that do not naturally occur in association with a ligand, but that are useful when manipulated into association with a ligand, that alone or in concert with other components enable the detection of a target, e.g., the biomarker LOX-1. Such labels or components include, without limitation, fluorescent agents, chemiluminescent agents, chromogenic agents, quenching agents, radionucleotides, enzymes, enzymatic substrates, cofactors, inhibitors, radioactive isotopes, magnetic particles, and other moieties known in the art. In certain embodiments, the “labels” or “reporter molecules” are covalently attached or associated with the ligand. In certain other embodiments, the “labels” or “reporter molecules” are non-covalently attached or associated with the ligand. Such labels are capable of generating a measurable signal alone, e.g., radioactivity, or in association with another component, e.g., an enzymatic signal in the presence of a substrate.

By “physical substrate is meant a substrate upon which said polynucleotides or oligonucleotides or ligands are immobilized. The physical substrate can be e.g., a glass slide, a plastic support, or a microchip. The term “microarray” refers to an ordered arrangement of binding/complexing array elements or ligands, e.g. antibodies, probes, etc. on a physical substrate.

By “significant change in expression” is meant an upregulation in the expression level of a nucleic acid sequence, e.g., genes or transcript, encoding a selected biomarker, in comparison to the selected reference standard or control; a downregulation in the expression level of a nucleic acid sequence, e.g., genes or transcript, encoding a selected biomarker, in comparison to the selected reference standard or control; or a combination of a pattern or relative pattern of certain upregulated and/or down regulated biomarker genes. The degree of change in biomarker expression can vary with each individual as stated above for protein biomarkers.

The term “polynucleotide,” when used in singular or plural form, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

One skilled in the art may readily reproduce the compositions and methods described herein by use of the amino acid sequences of the biomarkers and other molecular forms, which are publicly available from conventional sources.

Throughout this specification, the words “comprise”, “comprises”, and “comprising” are to be interpreted inclusively rather than exclusively. The words “consist”, “consisting”, and its variants, are to be interpreted exclusively, rather than inclusively. It should be understood that while various embodiments in the specification are presented using “comprising” language, under various circumstances, a related embodiment is also be described using “consisting of” or “consisting essentially of” language.

The term “a” or “an”, refers to one or more, for example, “a biomarker,” is understood to represent one or more biomarkers. As such, the terms “a” (or “an”), “one or more,” and “at least one” are used interchangeably herein.

As used herein, the term “about” means a variability of 10% from the reference given, unless otherwise specified.

### Methods

A method for differentiating polymorphonuclear myeloid derived suppressor cells (PMN-MDSCs) from polymorphonuclear neutrophils (PMNs) or monocytic myeloid derived suppressor cells (M-MDSCs) in a biological sample containing these types of cells involves the following steps. The biological sample, e.g., whole blood or a cell suspension, or a tumor exudate, or tissue, e.g., biopsy material, is contacted with a ligand that specifically binds or forms a complex with LOX-1 receptor on the cell surface. As described in the example below, the ligand is an antibody that binds to LOX-1. Thus, by contacting the sample with an anti-LOX-1 antibody, one may detect antibody-conjugate complexes in the sample. However, other ligands can be used in a similar fashion. The resulting complexes of ligand-bound LOX-1-cells in the sample are detected. Such detection can be based upon separation of the ligand-bound cells from unbound cells in the sample. The LOX-1-bound cells are PMN-MDSCs substantially free of PMN. In certain embodiments, the ligand is an anti-LOX-1 antibody, or an anti-LOX-1 antibody fragment. In certain embodiments, the ligands are associated with a detectable label component. In still other embodiments, the ligand is immobilized on a substrate.

In samples containing red blood cells, such as whole blood, one embodiment of the method involves killing or lysing the red blood cells to permit their elimination from the sample and possible interference with the results of the assay. In one aspect, the methods described herein comprise combining the whole blood sample with a lytic reagent system. This step can occur before contact of the sample with the ligand. In another embodiment, this step can occur after contact of the sample with the ligand. In still another embodiment, this step can occur simultaneously or substantially simultaneously with contact with the ligand. In such embodiments, the lytic reagent system is used to lyse red blood cells and to preserve the integrity of the remaining cells in the sample. Exemplary lytic reagents, stabilizing reagents and the method of use have been described, e.g., in U.S. Pat. Nos. 6,573,102 and 6,869,798. Alternatively, the reagent system can also be an isotonic lysing reagent as described in U.S. Pat. No. 5,882,934. Other lytic reagents known in the art can also be used for the purpose of the present methods.

The detection and separation of the ligand bound LOX-1 cells in the sample may be accomplished by a physical characteristic, such as the difference in size or weight of the bound LOX-1 cells vs. the unbound cells which do not have LOX-1 on their surfaces. Such detection and/or separation techniques can thus employ appropriately sized filtration units, or the use of flow cytometry, or chromatographic or centrifugation techniques (size exclusion or weight exclusion), among others known to the art.

Alternatively, where the ligand is associated with a detectable label component, the detection and separation may employ methods of detecting independently detectable labels by radioactivity, light wavelength, etc. Where the ligand is associated with a label which is capable of generating a measurable detectable signal when contacted with another label component, these methods employ the addition of such components and suitable detection methods dependent upon the signal generated. The separated, collected ligand-bound LOX-1⁺ cells are then collected and counted.

Where the ligand is immobilized on a physical substrate, the separating step can include washing the unbound cells and other debris in the sample from the substrate and counting or collecting the bound PMN-MDSCs from the substrate. In another embodiment, the separating step comprises treating the sample with a reagent, such as an enzymatic substrate, where the label is an enzyme. The interaction of the label and enzymatic substrate or cofactor identifies LOX-1-PMN-MDSC complexes from unbound cells to permit enumeration of PMN-MDSC.

The method of identifying and separating PMN-MDSCs from a sample can also include contacting the biological sample with the other biomarkers forming the distinguishing signature of PMN-MDSC or other biomarkers that identify as a single population both PMN-MDSCs and PMNs and/or M-MDSCs and isolating a cell suspension containing PMN-MDSCs and PMNs (and/or M-MDSCs) prior to, or simultaneously with, contacting the cell suspension with the LOX-1 ligand. In still other embodiments of the methods, the sample may be contacted (with or without RBC lysis) with a LOX-1 ligand and a ligand that identifies neutrophils, i.e., other PMN that are not LOX-1⁺. In one embodiment, the sample is contacted with a LOX-1 ligand and a CD15 ligand. In still other embodiments of the methods, the sample may be contacted with a LOX-1 ligand and a CD66b ligand. Still other ligands that identify neutrophils generally may be useful in this context.

In one embodiment, therefore, the method involves contacting the biological sample with the ligand for CD15 prior to, or simultaneously with, the use of the LOX-1 ligand. In one embodiment, therefore, the method involves contacting the biological sample with a ligand for CD66b prior to, or simultaneously with, the use of the LOX-1 ligand. In one embodiment, therefore, the method involves contacting the biological sample with a ligand for CD14 prior to, or simultaneously with, the use of the LOX-1 ligand. In one embodiment, therefore, the method involves contacting the biological sample with a ligand for CD11b prior to, or simultaneously with, the use of the LOX-1 ligand. In one embodiment, therefore, the method involves contacting the biological sample with the ligand for CD33, prior to, or simultaneously with, the use of the LOX-1 ligand. In one embodiment, therefore, the method involves contacting the biological sample with a ligand for CD14 and a ligand for CD15 prior to, or simultaneously with, the use of the LOX-1 ligand. In another embodiment, therefore, the method involves contacting the biological sample with a ligand for CD14, and a ligand for CD11b prior to, or simultaneously with, the use of the LOX-1 ligand. In another embodiment, therefore, the method involves contacting the biological sample with a ligand for CD14 and a ligand for CD33 prior to, or simultaneously with, the use of the LOX-1 ligand. In another embodiment, therefore, the method involves contacting the biological sample a ligand for CD15 and a ligand for CD11b prior to, or simultaneously with, the use of the LOX-1 ligand. In another embodiment, therefore, the method involves contacting the biological sample with a ligand for CD15 and a ligand for CD33 prior to, or simultaneously with, the use of the LOX-1 ligand. In another embodiment, therefore, the method involves contacting the biological sample with a ligand for CD15, a ligand for CD11b and a ligand for CD33 prior to, or simultaneously with, the use of the LOX-1 ligand. In another embodiment, therefore, the method involves contacting the biological sample with a ligand for CD14, a ligand for CD11b and a ligand for CD33 prior to, or simultaneously with, the use of the LOX-1 ligand.

In one embodiment of the method, any of these biomarkers may be detected prior to, or simultaneously with, the detection of the LOX-1 biomarker. The use of these other ligands assists in identifying all PMNs from other cells in the sample. Subsequent exposure of this population of cells from the sample with the LOX-1 ligands enables further separation of the PMN-MDSCs from the PMN population.

In one embodiment, following contact with the LOX-1 ligand and a second neutrophil specific biomarker ligand, such as a CD15 ligand or CD66b ligand, one may calculate the number of LOX-1⁺ vs. CD15⁺ or the number of LOX-1⁺ vs. CD66b⁺ cells are present in the sample. Such calculation can involve cell counting systems known to those of skill in the art.

In another embodiment, the method involves collecting as a second population, the cells which did not form complexes with the ligands, e.g., are not providing a detectable signal or are not immobilized on the substrate. This second population contains PMNs and other cells substantially free from PMN-MDSCs.

In still another embodiment, the methods described herein permit the obtaining of a population of cells enriched in human polymorphonuclear myeloid derived suppressor cells (PMN-MDSCs) by isolating from a cell suspension those cells which express LOX-1 to provide a population of cells enriched with PMN-MDSCs.

In still another embodiment, the methods involve measuring the amount of soluble LOX-1⁺ in the serum and correlating that number with the number of LOX-1⁺ PMN-MDSC.

These methods also permit the removal of human PMN-MDSCs from a cell population, comprising isolating from the cell population those cells which express LOX-1. These methods are useful in one embodiment for monitoring of the progression or metastasis of a cancer or the monitoring of therapy in a cancer patient by permitting the evaluation of an increase in the LOX-1 cell surface receptor in a biological sample of a patient having a cancer or under treatment for cancer. The increase of LOX-1+ cell number is indicative of metastasizing cancer or a progression of cancer. In other embodiments, this method may be useful diagnostically to initially detect the presence of cancer.

These methods depend initially upon obtaining an accurate enumeration or concentration of a PMN-MDSC cell population, substantially free of any PMNs, from a suitable biological sample of a subject. In one embodiment, these methods of determining an accurate cell count/concentration of cells expressing LOX-1 in a subject having a cancer or being treated for a cancer can be used to monitor the progression of the cancer (with or without treatment).

In still another embodiment, the use of these methods to determine an accurate measurement of LOX-1⁺ cells enable the monitoring of metastasis in a cancer, e.g., an increase in the LOX-1⁺ cell number indicates metastatic cancer. In another embodiment, these methods are useful to monitor and/or influence cancer treatment. For example, where the LOX-1+ cell number is increasing prior to cancer therapy, and subsequent performance of the method on a similar sample in the subject does not show a decrease in LOX-1+ cell number, the method can indicate that a change in therapeutic method or dosage is necessary.

In another embodiment, these methods of determining an accurate cell count/concentration of cells expressing LOX-1 in a subject suspected of having cancer, can diagnose the presence of cancer. In another embodiment, these methods can diagnose the aggressiveness of a cancer. In another embodiment, these methods can diagnose the stage of a cancer. According to the inventors' early studies, in most healthy individuals the proportion of LOX-1⁺ PMN is less than between 0.5% to 1% PMN. Patients with stage II diseases usually have between about 3 about 5% of LOX-1⁺ PMN and patients at stages III-IV have over 5% to about 12% PMN.

In still another aspect, the method of measuring the LOX-1⁺ population in a sample, such as whole blood, can be employed as a research method to determine the cause of the increase in such cells during the progression of a cancer.

In still other aspects of the diagnostics methods identified above, additional diagnostics steps include contacting the sample with a reagent that identifies activators or regulators of ER stress response in said cells. In one embodiment, the activators or regulators so identified are one or more of sXBP1, DDIT3 (CHOP), ATF4, ATF3, SEC61A ARGI or NOS-2. In another embodiment, the regulators are one or more of one or more of MYCN, CSF3, IL3, TGFβ1, TNF, LDL, RAF1, APP, IL6 PDGFBB, EPO, CD40LG, NFkB, IL13, AGT, IL1β, ERBB2, MAP2K1, VEGFα, CSF1, FLI1, or IFNγ.

Yet another embodiment of a diagnostic method for a mammalian subject with a cancer comprises the additional step of determining the size of a tumor in the subject by correlation with the number of LOX-1+ PMN or PMN-MDSC detected. This method step is further described in detail in the examples, but can include obtaining a biological sample from the subject; detecting whether soluble LOX-1 is present in the sample by contacting the sample with an antibody or functional antibody fragment that specifically binds or forms a complex with LOX-1 on the cell surface; and detecting and distinguishing the complexes of antibody-bound LOX-1-cells from other cells not bound to the antibody in the sample. The size of the tumor is then determined based upon the increase of LOX-1+ PMNs or PMN-MDSCs over a baseline level. The baseline level is readily determined based upon enumeration of patient samples to create a standard.

Still another method combines diagnosing and treating a cancer and combines the steps, such as obtaining a biological sample from a subject; detecting whether PMN-MDSC are present in the sample; diagnosing the subject with cancer when the presence of LOX-1+ (or any other of the PMN-MDSC signature biomarkers identified herein) is detected at a level that indicates PMN-MDSC are present; and administering an effective amount of a composition that reduces or inhibits ER stress response in mammalian neutrophils or reduces or inhibits LOX-1 expression on neutrophil populations.

The presence of LOX-1 (or any of the PMN-MDSC signature biomarkers) in the sample (or a LOX-1-ligand complex) may be detected using any assay format known in the art or described herein. There are a variety of assay formats known to the skilled artisan for using a ligand to detect a target molecule in a sample. (For example, see Harlow and Lane, Antibodies: A Laboratory Manual, Cold Spring Harbor Laboratory, 1988). In general, the presence or absence of LOX-1 in a sample may be determined by (a) contacting the sample with a ligand that interacts with LOX-1; and (b) determining the presence or level of LOX-1 in the sample, wherein the presence of LOX-1 in the sample is indicative of cancer or where an increase in the level of LOX-1 in the sample as compared to a control, is indicative of cancer. The various assay methods employ one or more of the LOX-1-binding ligands described herein, e.g., polypeptide, polynucleotide, and/or antibody, which detect the LOX-1 protein or mRNA encoding the same (including fragments or portions thereof).

Methods of detection, diagnosis, monitoring, and prognosis of cancer, or the status of cancer, and for the identification of subjects with an increased risk of cancer metastasis by detecting the presence of, or measuring the level of LOX-1 protein or another biomarker described herein, are provided herein. Such methods may employ polypeptides and/or antibodies as described herein. The particular assay format used to measure the LOX-1 in a biological sample may be selected from among a wide range of immunoassays, such as enzyme-linked immunoassays, sandwich immunoassays, homogeneous assays, immunohistochemistry formats, or other conventional assay formats. One of skill in the art may readily select from any number of conventional immunoassay formats to perform this invention. Other reagents for the detection of protein in biological samples, such as peptide mimetics, synthetic chemical compounds capable of detecting LOX-1 may be used in other assay formats for the quantitative detection of LOX-1 protein in biological samples, such as high pressure liquid chromatography (HPLC), immunohistochemistry, etc.

Methods of detection, diagnosis, monitoring, and prognosis of cancer, or the status of cancer, and for the identification of subjects with an increased risk of cancer metastasis by detecting the presence of, or measuring the level of LOX-1 mRNA, are provided herein. Such methods include methods based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as reverse transcription polymerase chain reaction (RT-PCR) or qPCR.

Such PCR-based method may employ a primer or primer-probe set capable of identifying and/or amplifying a LOX-1 nucleic acid sequence or a portion thereof. An example of a primer set capable of identifying and/or amplifying a LOX-1 nucleic acid sequence or a portion thereof is described in Example 1E. Such primers include those described in the examples or other suitable primers can be designed by the person of skill in the art and/or obtained commercially based on the LOX-1 nucleic acid sequence.

The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, Calif.), differential display, amplified fragment length polymorphism (iAFLP), and BeadArray™ technology (Illumina, San Diego, Calif.) using the commercially available Luminex100 LabMAP system and multiple color-coded microspheres (Luminex Corp., Austin, Tex.) and high coverage expression profiling (HiCEP) analysis.

The diagnostic methods described herein can employ contacting a patient's sample with a diagnostic reagent, as described above, which forms a complex or association with LOX-1 in the patients' sample. Detection or measurement of the sample LOX-1 may be obtained by use of a variety of apparatus or machines, such as computer-programmed instruments that can transform the detectable signals generated from the diagnostic reagents complexed with the LOX-1 or other biomarker in the biological sample into numerical or graphical data useful in performing the diagnosis. Such instruments may be suitably programmed to permit the comparison of the measured LOX-1 in the sample with the appropriate reference standard and generate a diagnostic report or graph.

The selection of the polynucleotide sequences, their length and labels used in the composition are routine determinations made by one of skill in the art in view of the teachings of which genes can form the gene expression profiles suitable for the diagnosis and prognosis of cancer. For example, useful primer or probe sequences can be at least 8, at least 10, at least 15, at least 20, at least 30, at least 40 and over at least 50 nucleotides in length. For example, such probes and polynucleotides can be complementary to portions of mRNA sequences encoding LOX-1 or another of the biomarkers identified herein. The probes and primers can be at least 70%, at least 80%, at least 90%, at least 95%, up to 100% complementary to sequences encoding.

In any of the methods described herein, in one embodiment, the sample comprises blood, plasma or cells. Such sample may be derived from a tissue biopsy. In some of the methods described herein, a control level is used as a reference point. The control level can be any of those described herein. In one embodiment, the control level is the level obtained from an individual, or a population of individuals, who are healthy (i.e., who do not have cancer). In another embodiment, the control level is the level obtained from an individual, or a population of individuals, who have cancer that has not metastasized.

Compositions

In yet another embodiment, the methods described above result in a composition of cells, i.e., a substantially pure population of PMN-DMSCs produced by isolating LOX-1⁺ cells from a biological sample by contacting the sample with a reagent that forms a complex or binds to LOX-1. The methods described above can also result in a population of PMNs which contain substantially no PMN-DMSCs. These cell populations are useful in research.

In a further aspect, diagnostic composition or kit is provided by the disclosures and experiments described herein. In one embodiment, such a composition comprises a ligand that specifically binds or forms a complex with LOX-1 on the cell surface. Such a composition may include ligands and antibodies and small molecules that can detecting or isolate a population of human polymorphonuclear myeloid derived suppressor cells (PMN-MDSCs). Such useful compositions include anti-LOX-1 antibodies or small molecules that can bind thereto. Also useful are antibodies or ligands that bind other of the genes that form the genetic signature of the PMN-DMSCs, such as the genes identified in FIG. 9A and FIG. 10B, as well as the top ranked 985 genes that differentiate between PMN-MDSC and PMN listed in Table 1, disclosed above.

Included among them are markers and regulators of pathways for ER stress response, such as sXBP1, DDIT3 (CHOP), ATF4, ATF3, SEC61A ARGI, NOS-2, MYCN, CSF3, IL3, TGFβ1, TNF, LDL, RAF1, APP, IL6 PDGFBB, EPO, CD40LG, NFkB, IL13, AGT, IL1β, ERBB2, MAP2K1, VEGFα, CSF1, FLI1, or IFNγ. Still other likely biomarkers for pathways involved or activated in PMN-MDSC production are described in the Examples below. This composition/kit containing ligands/antibodies or small molecules that bind to one or a combination of any of these biomarkers may be used in diagnosing the presence, progression or metastasis of a cancer.

A variety of compositions and methods can be employed for the detection, diagnosis, monitoring, and prognosis of the relevant cancer, or the status of cancer, and for the identification of subjects with an increased risk of cancer metastasis. The cancer may be any one of the cancers described in Tables 3 and 4 below, for example. In one aspect, a diagnostic composition useful in diagnosing and/or treating cancer is provided. In one embodiment, the composition includes a ligand which is capable of specifically complexing with, or identifying, LOX-1 or the other biomarkers that together with LOX-1 can differentiate non-immunosuppressive neutrophils or LOX-1− PMNs from immunosuppressive PMN-MDSCs or subsets thereof, or the mRNA encoding the same, including a fragment or portion thereof.

There are a variety of assay formats known to the skilled artisan for using a binding agent to detect a target molecule in a sample. Any ligand which is capable of specifically complexing with, or identifying, the relevant biomarker, the mRNA encoding the same, including a fragment or portion thereof, which is useful in one or more of the various assay methods, is contemplated herein. In one embodiment, the ligand is a polynucleotide or oligonucleotide sequence, which sequence binds to, complexes with or identifies LOX-1 (or any of the biomarkers forming the PMN-MDSC signature) or the mRNA encoding the same, or a fragment thereof. In another embodiment, the ligand is a protein or peptide, which protein or peptide binds to, complexes with or identifies LOX-1 (or any of the biomarkers forming the PMN-MDSC signature) or the mRNA encoding the same or a portion or fragment thereof. In another embodiment, the ligand is an antibody or fragment thereof which binds to, complexes with or identifies LOX-1 (or any of the biomarkers forming the PMN-MDSC signature) or the mRNA encoding the same or a portion or fragment thereof.

The terms antibody and antibody fragment are defined above. A recombinant molecule bearing the binding portion of an anti-LOX-1 antibody (or another molecule designed similarly to target one or more of the PMN-MDSC biomarkers referred to herein, or the ER stress response proteins or genes), e.g., carrying one or more variable chain CDR sequences that bind LOX-1 or the other target, may also be used in a diagnostic assay. As used herein, the term “antibody” may also refer, where appropriate, to a mixture of different antibodies or antibody fragments that bind to LOX-1 or anoather selected target disclosed herein. Such different antibodies may bind to different biomarkers in the PMN-MDSC signature or different portions of LOX-1 protein than the other antibodies in the mixture.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. Preferably, an anti-biomarker antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+ PECy5 and PE+ PECy7, among others may be used depending upon assay method.

In yet another embodiment, the reagent is a primer set or primer-probe set capable of identifying and/or amplifying LOX-1 or a portion thereof or any of the other biomarkers discussed herein. An example of a primer set capable of identifying and/or amplifying such a biomarker or a portion thereof is described in the examples below. Other suitable primers can be designed by the person of skill in the art and/or obtained commercially.

In one embodiment, the reagent forms a complex with LOX-1. In one embodiment, the reagent-LOX-1 complex is capable of being detected. Various methods of detection of the reagent-LOX-1 complex are known in the art. In some embodiments, such methods include the use of labels as described herein.

In one embodiment, the ligand is associated with a detectable label or a substrate. The ligand may be covalently or non-covalently joined with the detectable label or substrate. In one embodiment, the comprises a substrate upon which said ligand is immobilized. For these reagents, the labels may be selected from among many known diagnostic labels, including those described above. Selection and/or generation of suitable ligands with optional labels for use in this invention is within the skill of the art, provided with this specification, the documents incorporated herein, and the conventional teachings of the art. Ligands may be labeled using conventional methods with a detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol, enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags).

Similarly, the substrates for immobilization may be any of the common substrates, glass, plastic, a microarray, a microfluidics card, a chip or a chamber. The reagent itself may be labeled or immobilized. For example, a ligand or sample may be immobilized on a carrier or solid support which is capable of immobilizing cells, antibodies, etc. Suitable carriers or supports may comprise nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip) Immobilization typically entails separating the binding agent from any free analytes (e.g. free markers or free complexes thereof) in the reaction mixture.

Still another diagnostic reagent includes a composition or kit comprising at least one reagent that binds to, hybridizes with or amplifies LOX-1 or any of the other PMN-MDSC signature biomarkers. Such diagnostic reagents and kits containing them are useful for the measurement and detection of the biomarkers in the methods described herein for diagnosis/prognosis of cancer or metastasis of cancer. In addition to the reagents above, alternatively, a diagnostic kit thus also contains miscellaneous reagents and apparatus for reading labels, e.g., certain substrates that interact with an enzymatic label to produce a color signal, etc., apparatus for taking blood samples, as well as appropriate vials and other diagnostic assay components.

In yet another aspect, a pharmaceutical composition is provided that reduces or inhibits ER stress in mammalian neutrophils or reduces or inhibits LOX-1 expression on neutrophil populations in a pharmaceutically acceptable carrier or excipient. In one embodiment, this composition comprises an antagonist or inhibitor of the expression, activity or activation of one or more of sXBP1, DDIT3 (CHOP), ATF4, ATF3, SEC61A ARGI or NOS-2. In one embodiment, the composition comprises an antagonist or inhibitor of LOX-1. In still further embodiments, the composition contains additional antagonist or inhibitor of the expression, activity or activation of one or more of MYCN, CSF3, IL3, TGFβ1, TNF, LDL, RAF1, APP, IL6 PDGFBB, EPO, CD40LG, NFkB, IL13, AGT, IL1β, ERBB2, MAP2K1, VEGFα, CSF1, FLI1, or IFNγ, or of the pathways leading to the production of the immunosuppressive PMN-MDSC populations in vivo.

In one embodiment, the antagonist or inhibitor of the selected mediator of ER stress is an antibody, functional antibody fragment or equivalent as defined herein, or a similarly functioning small molecule that binds to and thus prevents the normal activity of the particular gene/protein described above, leading to a reduction of the ER stress induction to which the neutrophils are exposed.

As another aspect, a novel pharmaceutical composition comprises the antagonist or inhibitors and immunotherapeutics described above in a pharmaceutically acceptable carrier or excipient in an effective amount to reduce, inhibit, retain or suppress growth of the PMN-MDSC population. In one aspect, the pharmaceutical composition contains, e.g., about 25, 30, 35, 40, 45, 50, 55, 60, 65, 70, 75, 80, 85, to about 90% of the antagonist or inhibitors in combination with a pharmaceutical carrier or excipient.

By “pharmaceutically acceptable carrier or excipient” is meant a solid and/or liquid carrier, in in dry or liquid form and pharmaceutically acceptable. The compositions are typically sterile solutions or suspensions. Examples of excipients which may be combined with the antagonist or inhibitor include, without limitation, solid carriers, liquid carriers, adjuvants, amino acids (glycine, glutamine, asparagine, arginine, lysine), antioxidants (ascorbic acid, sodium sulfite or sodium hydrogen-sulfite), binders (gum tragacanth, acacia, starch, gelatin, polyglycolic acid, polylactic acid, poly-d,l-lactide/glycolide, polyoxaethylene, polyoxapropylene, polyacrylamides, polymaleic acid, polymaleic esters, polymaleic amides, polyacrylic acid, polyacrylic esters, polyvinylalcohols, polyvinylesters, polyvinylethers, polyvinylimidazole, polyvinylpyrrolidon, or chitosan), buffers (borate, bicarbonate, Tris-HCl, citrates, phosphates or other organic acids), bulking agents (mannitol or glycine), carbohydrates (such as glucose, mannose, or dextrins), clarifiers, coatings (gelatin, wax, shellac, sugar or other biological degradable polymers), coloring agents, complexing agents (caffeine, polyvinylpyrrolidone, β-cyclodextrin or hydroxypropyl-β-cyclodextrin), compression aids, diluents, disintegrants, dyes, emulsifiers, emollients, encapsulating materials, fillers, flavoring agents (peppermint or oil of wintergreen or fruit flavor), glidants, granulating agents, lubricants, metal chelators (ethylenediamine tetraacetic acid (EDTA)), osmo-regulators, pH adjustors, preservatives (benzalkonium chloride, benzoic acid, salicylic acid, thimerosal, phenethyl alcohol, methylparaben, propylparaben, chlorhexidine, sorbic acid, hydrogen peroxide, chlorobutanol, phenol or thimerosal), solubilizers, sorbents, stabilizers, sterilizer, suspending agent, sweeteners (mannitol, sorbitol, sucrose, glucose, mannose, dextrins, lactose or aspartame), surfactants, syrup, thickening agents, tonicity enhancing agents (sodium or potassium chloride) or viscosity regulators. See, the excipients in “Handbook of Pharmaceutical Excipients”, 5^{th} Edition, Eds.: Rowe, Sheskey, and Owen, APhA Publications (Washington, D.C.), 2005 and U.S. Pat. No. 7,078,053, which are incorporated herein by reference. The selection of the particular excipient is dependent on the nature of the compound selected and the particular form of administration desired.

Solid carriers include, without limitation, starch, lactose, dicalcium phosphate, microcrystalline cellulose, sucrose and kaolin, calcium carbonate, sodium carbonate, bicarbonate, lactose, calcium phosphate, gelatin, magnesium stearate, stearic acid, or talc. Fluid carriers without limitation, water, e.g., sterile water, Ringer's solution, isotonic sodium chloride solution, neutral buffered saline, saline mixed with serum albumin, organic solvents (such as ethanol, glycerol, propylene glycol, liquid polyethylene glycol, dimethylsulfoxide (DMSO)), oils (vegetable oils such as fractionated coconut oil, arachis oil, corn oil, peanut oil, and sesame oil; oily esters such as ethyl oleate and isopropyl myristate; and any bland fixed oil including synthetic mono- or diglycerides), fats, fatty acids (include, without limitation, oleic acid find use in the preparation of injectables), cellulose derivatives such as sodium carboxymethyl cellulose, and/or surfactants.

By “effective amount” is meant the amount or concentration (by single dose or in a dosage regimen delivered per day) of the antagonist or inhibitor sufficient to retard, suppress or inhibit the PMN-MDSC, while providing the least negative side effects to the treated subject. One of skill in the art would be able to determine the amount of these antagonist or inhibitors to administer alone or in combination with an additional reagent, e.g., chemotherapeutic, antibiotic or the like. In a further embodiment, the combination of the antagonist or inhibitors with another pharmacological agent or treatment protocol permits lower than usual amounts of the agonist and additional chemotherapeutic agent to achieve the desired therapeutic effect. In another embodiment, the combination of the antagonist or inhibitors with another chemotherapy treatment protocol permits adjustment of the additional protocol regimen to achieve the desired therapeutic effect.

In one embodiment, the effective amount of the antagonist or inhibitors is within the range of 1 mg/kg body weight to 100 mg/kg body weight in humans including all integers or fractional amounts within the range. In certain embodiments, the effective amount is at least 2, 5, 10, 15, 20, 25, 30, 35, 40, 45, 50, 55, 60, 65, 70, 75, 80, 85, 90, 95 or 100 mg/kg body weight, including all integers or fractional amounts within the range. In one embodiment, the above amounts represent a single dose. In another embodiment, the above amounts define an amount delivered to the subject per day. In another embodiment, the above amounts define an amount delivered to the subject per day in multiple doses. In still other embodiments, these amounts represent the amount delivered to the subject over more than a single day.

In another embodiment, the pharmaceutical composition contains a LOX-1 or ER response antagonist or inhibitor and a chemotherapeutic. Alternatively, the active compound is formulated with a chemotherapeutic for treatment of the cancers described herein. In one embodiment, the chemotherapeutic is selected from among those described above. Alternatively, the composition is formulated with another effective compound or reagent for treatment of the cancers described herein, such as an antibiotic or bactericide, a surfactant, or other reagent commonly used in formulation of anti-cancer compositions.

The forms of the pharmaceutical compositions may be liquid, solid or a suspension or semi-solid and designed for use with a desired administrative route, such as those described herein. The doses and dosage regimens are adjusted for the particular cancer, and the stage of the cancer, physical status of the subject. Such doses may range from about 1 to about 100 mg/kg subject body weight of the antagonist or inhibitor, as discussed above and include dosage regimens designed to administer the effective amount in smaller repeated doses.

These compositions are useful in methods for treating any cancer including the cancers described herein and in the examples. In still another embodiment, a therapeutic method for reducing or inhibiting LOX-1+ PMN-MDSC accumulation in a cancer patient comprises administering a composition such as described herein at a suitable dosage. This reduction can be for the treatment of cancer alone. In still other embodiments, the treatment step may be combined with the diagnostic steps in a combined method.

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples, and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

### Example 1: Identifying Discriminatory Markers

In order to identify specific markers discriminating between these two populations, we performed genome—wide microarrays (Human HT-12 v4 expression Beadchip, Illumina) to compare the gene expression profiles between PMN-MDSC and PMN from the same cancer patients (7 patients) as well as age matching healthy donors (4 donors). All samples of peripheral blood (PB) were collected from patients at the Helen F. Graham Cancer Center and were analyzed within 3 hours of collection. PMN-MDSCs were evaluated in mononuclear fraction of PB after ficoll density gradient. PMN were evaluated from the cell fraction remaining after removal of mononuclear cells. Cells were resuspended in PBS and loaded on a step density gradient (Percoll 63% on top of Percoll 72%) to separate PMNs in a monolayer between the two Percoll phases. In an attempt to minimize the number of potential candidates and to identify true marker of PMN-MDSC, we analyzed the gene expression profiles of PMN-MDSC from head and neck cancer patients (4 samples) as well as lung cancer patients (3 samples).

The analysis was performed using SAM analysis (significant analysis of microarray) and the false discovery rate set at 5% (analysis was performed by the Wistar bioinformatics core facility). This analysis allowed us to identify more than 1500 genes showing a significant differential expression between PMN-MDSC and PMN. The vast majority of the differentially regulated genes were up-regulated in PMN-MDSC compared to PMN. After filtering for molecules expressed on the surface of the cells, we ended with a relatively small list of specific biomarkers for PMN-MDSC. One of these biomarkers is the Lectin-like oxidized low-density lipoprotein receptor-1 (LOX-1), a 50 kDa transmembrane glycoprotein encoded by the gene olr1 (oxidized LDL receptor 1). According to the microarray, LOX-1 was increased by 5.75-fold in PMN-MDSC compared to PMN.

### Example 2—Confirming Validity of Lox-1 as a Biomarker

To confirm the validity of LOX-1 as a potential biomarker of PMN-MDSC, we analyzed the expression of this receptor by flow cytometry using an anti-LOX-1 monoclonal antibody (clone 15C4; Biolegend Inc., San Diego, Calif.) in blood samples from patients with 4 different types of cancer: head and neck, breast, non-small lung, or colon cancer.

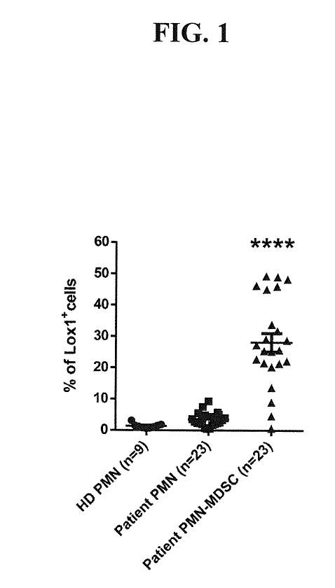

We first analyzed the expression of LOX-1 using the classical definitions of PMN-MDSC (CD11b⁺CD14⁻CD15⁺ and CD33⁺ from the low density mononuclear cells fraction) and PMN (cells with the same phenotype from high density fraction). The results of this experiment are reported graphically when healthy donors (HD) were compared with all cancer patients in FIG. 1. About 30% of the PMN-MDSC from all cancer patients (n=23) was found to express LOX-1 on their surface compared to less than 3% of the PMN from matching patients or about 1% from PMN from healthy donor (n=9) (p<0.001).

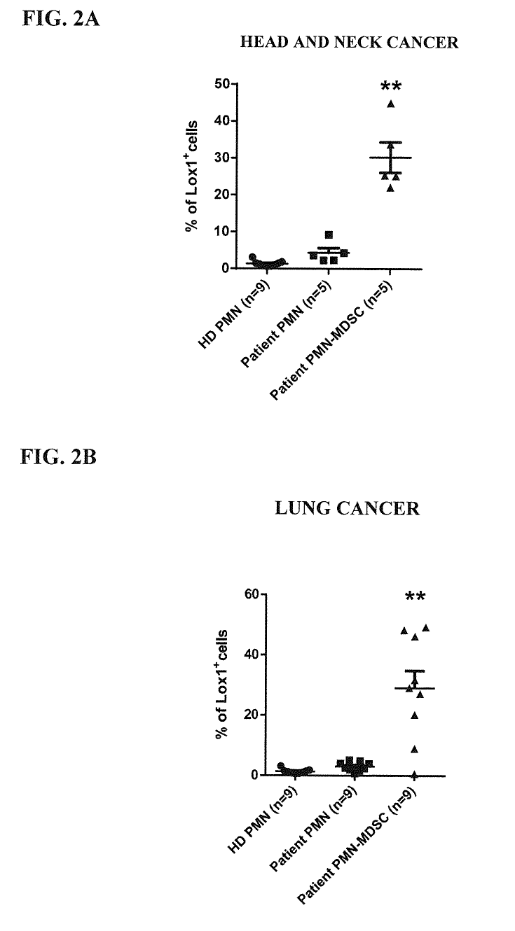

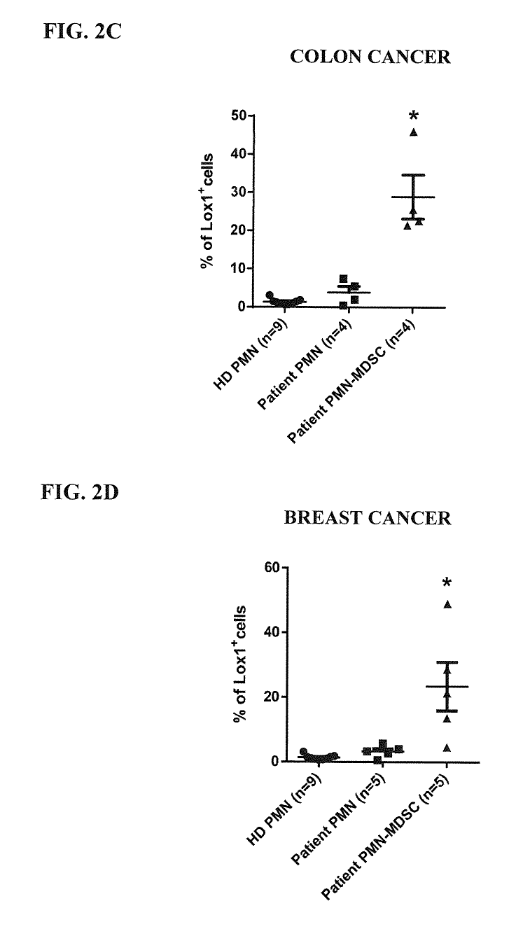

The results of this experiment are reported by separating the results for cancer types as shown in the graphs of FIGS. 2A through 2D. The results in all 4 types of cancer were similar.

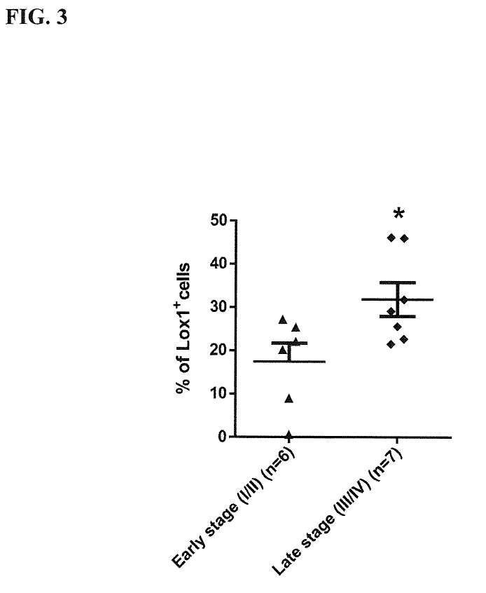

Preliminary data also suggest that the percentage of PMN-MDSC expressing LOX-1 could correlate with the stage of the disease. As shown graphically in the preliminary analysis of FIG. 3, only 20% of the PMN-MDSC from samples from early stage cancer patients expresses LOX-1 in comparison to 32% in samples from late stage cancer patients (p<0.05).

### Example 3—Analysis of Whole Blood Samples

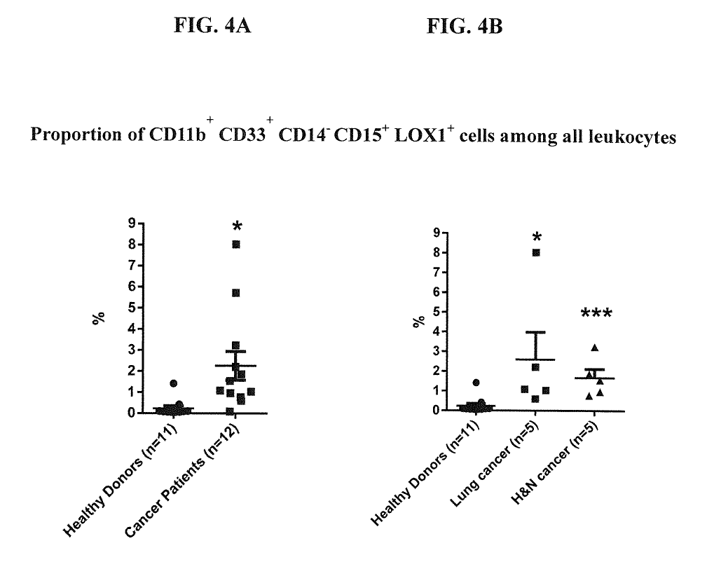

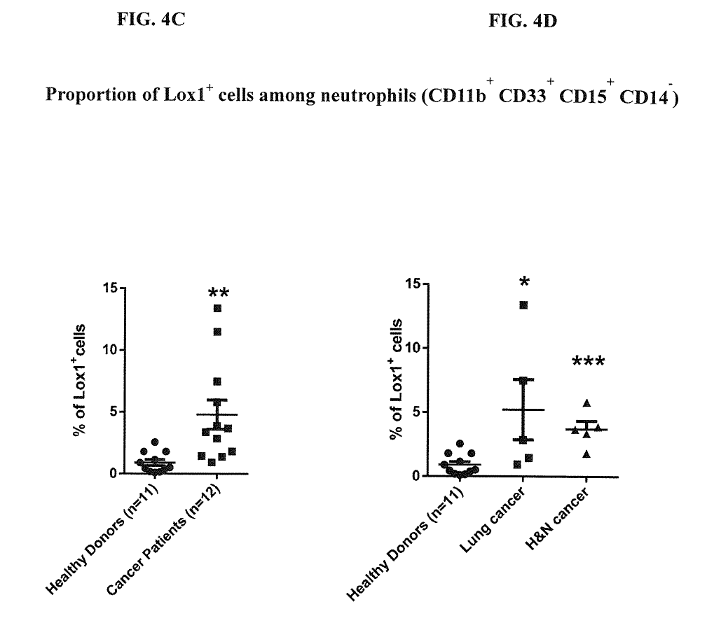

We also performed an analysis of unseparated whole blood samples. As shown in FIGS. 4A and 4C, as expected, about 1% or less of the CD11b⁺CD14⁻CD15⁺ and CD33⁺ PMN from healthy donors expressed LOX-1 on their surface. However, in samples from cancer patients (both head and neck and lung cancer patients), almost 5% of the PMN types of cells exhibit a positive staining for LOX-1 (≈2.3% of the total leukocytes) strongly supporting the designation of LOX-1 as a specific marker of PMN-MDSC. These results were confirmed by analyzing the disease separately, as reported in FIGS. 4B and 4D.

### Example 4—Stimulation of T-Cell Proliferation

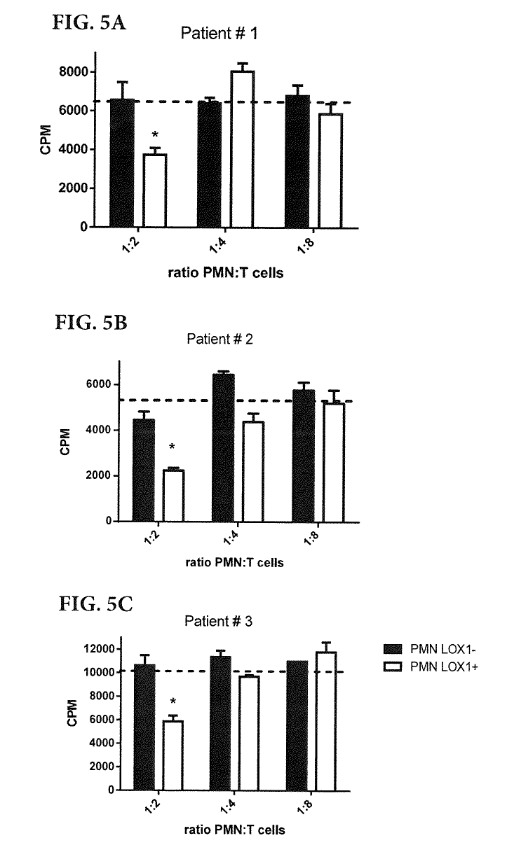

To assess possible functional relevance of these findings, LOX-1+ and LOX-1⁻ PMN were isolated from peripheral blood of three patients with head and neck cancer using magnetic beads separation as follows: Samples of whole blood were collected from patient with HNC. Red cells were lysed, and PMN were highly enriched by negative selection using Mitlenyi bead kit. Cells were then labeled with biotinylated LOX-1 antibody followed by streptavidin beads. LOX-1⁺ and LOX-1⁻ PMN were added to mixed allogeneic reaction at ratios if 1:2, 1:4 and 1:8, and T-cell proliferation was measured 5 days later by ³H-thymidin uptake. Experiments were performed in triplicate. Cells were used in allogeneic mixed leukocyte reactions where dendritic cells from healthy donors were cultured with T cells from unrelated healthy donors. Mixing cells from unrelated donors stimulated potent T-cell proliferation. As in shown in FIGS. 5A through 5C, the addition of LOX-1− PMN did not affect T-cell proliferation, whereas LOX-1+ PMN potently suppress T-cell response.

### Example 5—Evaluation of ROS

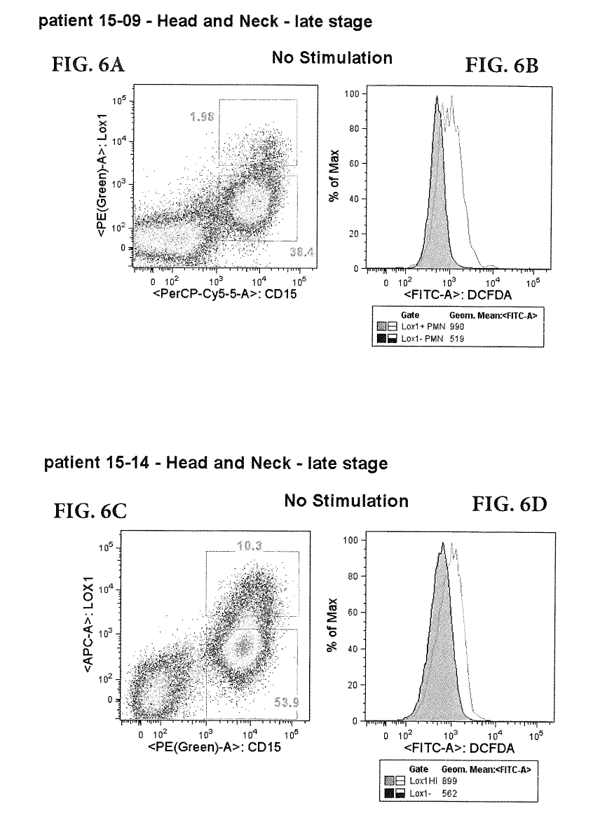

Reactive oxygen species (ROS) are considered as major mechanism responsible for immune suppressive activity of PMN-MDSC. We evaluated the level of ROS in LOX-1+ and LOX-1− PMN in patients with head and neck cancers (HNC) as follows. Samples of whole blood were collected from head and neck cancer patients. Red cells were lysed and PMN were labeled with CD15, LOX-1 antibodies, and with the cell permeant reagent 2′,7′-dichlorofluorescin diacetate (DCFDA). DCFDA is a fluorogenic dye that measures hydroxyl, peroxyl and other reactive oxygen species (ROS) activity within the cell. After diffusion in to the cell, DCFDA is deacetylated by cellular esterases to a non-fluorescent compound, which is later oxidized by ROS into 2′,7′-dichlorofluorescein (DCF). DCF is a highly fluorescent compound which can be detected by fluorescence spectroscopy with maximum excitation and emission spectra of 495 nm and 529 nm respectively. As shown in the histograms of FIGS. 6A-6D, LOX-1⁺ PMN had almost two-fold higher amount of ROS than LOX-1− PMN.

### Example 6—Correlation of Soluble LOX-1 with PMN-MDSC

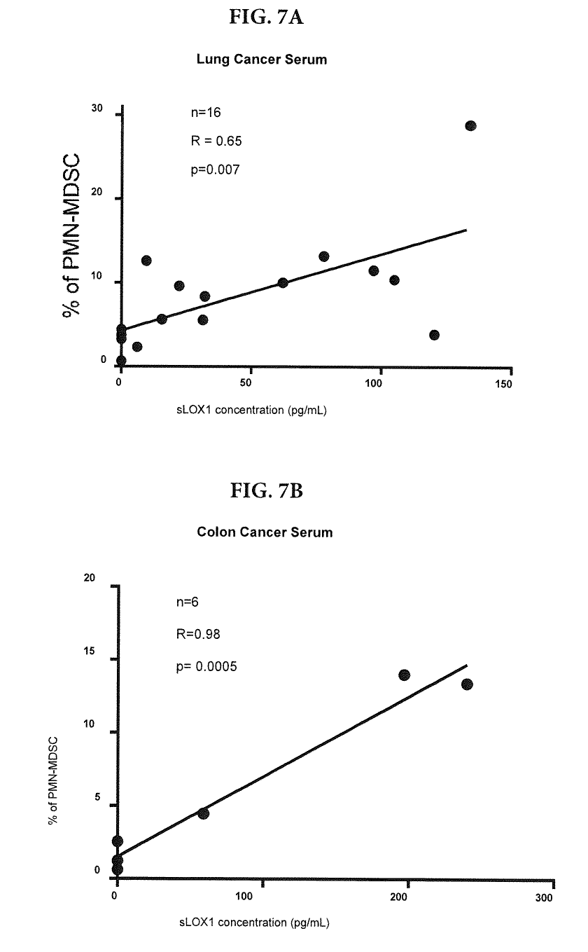

LOX-1 is known to be cleaved from the surface of the cells and can be detected in sera of patients. We hypothesized that LOX-1 may be cleaved from PMN-MDSC and therefore, the presence of soluble LOX-1 (sLOX-1) may correlate with the amount of LOX-1⁺ PMN-MDSC. Concentrations of sLOX-1 were measured in sera of 16 lung cancer patients and 6 colon cancer patients using ELISA. Samples of whole blood were collected; PBMC were purified using Ficoll gradient; and the proportion of PMN-MDSC out of total live PBMC was measured by flow cytometry using antibodies to CD11b, CD33, CD14, and CD15. The correlation between the presence of PMN-MDSC and soluble LOX-1 in sera of the lung cancer patients is shown in FIG. 7A and for colon cancer patients in FIG. 7B.

Highly significant correlation between these two parameters was found (correlation of coefficient in lung cancer patients 0.65, p=0.007; in patients with colon cancer 0.98, p=0.0005).

### Example 7—Methods and Materials

The following Examples 8-13 employ one or more of these methods and materials:

Human Samples:

Samples of peripheral blood and tumor tissues were collected from patients at Helen F. Graham Cancer Center and University of Pennsylvania. The study was approved by Institutional Review Boards of the Christiana Care Health System at the Helen F. Graham Cancer Center, University of Pennsylvania and The Wistar Institute. All patients signed approved consent forms. Peripheral blood was collected from:
1) 26 patients with different stages of non-small cell lung cancer (NSCLC)—12 females, 18 males, age 59-79 (median 69). 13 patients had squamous cell carcinoma, 13 patients—adenocarcinoma;
2) 21 patients with head and neck cancer (HNC)—8 females, 13 males, age 32-82 (median 65). 19 patients had squamous cell carcinoma and 2 patients—adenocarcinoma;
3) 38 patients with colon cancer (CC) (adenocarcinoma)—20 females, 18 males, age 28-88 (median 58).
4). 6 patients with multiple myeloma (MM)—1 female, 5 males, age 58-81 (median 75).

All patients were either previously untreated or received treatment (chemotherapy or radiation therapy) at least 6 months before collection of blood. In some patients, tumor tissues were collected during the surgery. In addition, 6 patients with eosinophilic colitis, 3 patients with ulcerative colitis, and 8 patients with Crohn's disease were evaluated.

Peripheral samples of blood from 18 healthy volunteers 12 females, 6 males age 35-56 (median 42 years) were used as control.

Lung cancer tumor microarrays were produced from formalin-fixed paraffin embedded tissue. Each block was examined by a pathologist and three cores were obtained from tumor-containing areas and three blocks from non-tumor involved lung regions. Samples were obtained from 32 patients with adenocarcinoma. Clinical data obtained included tumor histology, size, stage, and time to recurrence (all patients followed for 5 years).

De-identified samples from normal colonic biopsies colon were obtained from St. Mark's Hospital, Harrow, UK. Samples were taken from patients after obtaining informed consent and with the approval of the Outer West London Research Ethics Committee (UK). Paraffin-embedded tissue blocks of samples of normal skin, lymph nodes, and melanoma were retrieved using an approved IRB protocol for de-identified archived skin biopsies through the Department of Dermatology NIH SDRC Tissue Acquisition Core (P30-AR057217), Perelman School of Medicine, University of Pennsylvania, Philadelphia, USA.

Cell Isolation and Culture:

PMN-MDSC and PMN were isolated by centrifugation over a double density gradient Histopaque (Sigma) (1.077 to collect PBMC and 1.119 to collect PMN) followed by labeling with CD15-PE mAb (BD Biosciences) and then separated using anti-PE beads and MACS column (Miltenyi). Tissues were first digested with human tumor dissociation kit (Miltenyi) and then red blood cell lysed. Cells were then culture in RPMI (Biosource International) supplemented with 10% FBS, 5 mM glutamine, 25 mM HEPES, 50 μM β-mercaptoethanol and 1% antibiotics (Invitrogen). In some experiments, recombinant GM-CSF (Peprotech) was added to the culture media at a concentration of 10 ng/mL.

Isolation of LOX1+ PMN from Peripheral Blood and Suppression Assay:

Whole blood was enriched for PMNs using MACSxpress® Neutrophil Isolation Kit (Miltenyi) following the protocol provided by the manufacturer. Cells were then labeled with anti-Lox1-PE mAb (Biolegend) and then separated using anti-PE beads and MACS column (Miltenyi). For the three-way allogenic MLR suppression assay, T lymphocytes from one healthy donor were purified using the Human CD3+ T Cell Enrichment Column Kit (R&D Systems) and used as responder cells. Dendritic cells were generated from adherent monocytes from another healthy donor in the presence of 25 ng/mL GM-CSF and 25 ng/mL IL-4 (Peprotech) for 6 days and used as stimulator cells. Responder and stimulator cells were then mixed at a 10:1 ratio followed by the addition of Lox1+ or Lox1− PMNs. T lymphocyte proliferation was assessed after 5 days of culture by thymidine incorporation.

Concurrently, T-lymphocytes were isolated from the PBMC of the same patient as LOX-1+ PMN using human CD3+ T Cell Enrichment Column Kit. PMNs were plated at different ratios with 105 T lymphocytes in a 96-well plate coated with 10 μg/ml anti-CD3 (clone UCHT1; BD Biosciences) followed by the addition of 1 μg/ml of soluble anti-CD28 (clone CD28.2; BD Biosciences). T lymphocyte proliferation was assessed after 3 days of culture by thymidine incorporation.

In some experiments, 1 μM N-acetyl cysteine (NAC; Sigma) or 20 μM of Nω-hydroxy-norarginine (nor-NOHA; Cayman Chemical) was added to the culture media to block ROS or agrinase I activity, respectively. T lymphocyte proliferation was assessed after 5 days of culture by thymidine incorporation.

In Vitro PMN LOX-1 Induction:

PMNs from healthy donors were isolated on a Histopaque gradient. 5×10⁵ cells/ml were cultured for 12 hrs with 10 ng/ml of GM-CSF in the presence of dithiothreitol (DTT) (0.5, 1, 2 mM; Sigma), tunicamycin (0.5, 1, and 2 μg/ml; Sigma-Aldrich), or thapsigargin (0.5, 1, and 2 μM; Sigma). In some instances, 20 μM of the XBP-1 inhibitor BIO9 was added 3 hours prior to culture. Cells were then stained for flow cytometry or used for functional assays as described above.

Flow Cytometry:

Flow cytometry data were acquired using a BD LSR II flow cytometer and analyzed using FlowJo software (Tree Star) Immunofluorescent microscopy: Following deparaffinization and rehydration, heat induced antigen retrieval was performed using Tris-EDTA buffer pH 9. Followed by blocking with 5% BSA, tissues were stained with Lox1 antibody (Abcam; Cat no ab126538) and CD15 antibody (BD biosciences; Cat no 555400) at 1:200 dilution in 5% BSA each for 1 hour at room temperature. The following secondary antibodies were used Alexa Fluor anti-rabbit A647 (1:200 dilution in 5% BSA, Life technologies) for Lox1 and anti-mouse A514 (1:400 dilution in 5% BSA, Life technologies) for CD15 staining. CD15 staining was pseudo colored red and Lox1 staining was pseudo colored green. Nucleus was stained using DAPI (1:5000 dilution in PBS, Life technologies). Images were obtained using Leica TCS SP5 Confocal microscope. Cell counts from 16 frames were used to calculate counts per sq. mm.

Microarray Analysis:

For sample preparation and hybridization, total RNA from purified cells was isolated with TRIzol reagent according to the manufacturer's recommendations. RNA quality was assessed with a Bioanalyzer (Agilent). Only samples with RIN numbers>8 were used. Equal amount (400 ng) of total RNA was amplified as recommended by Illumina and was hybridized to the Illumina HumanHT-12 v4 human whole-genome bead arrays.

For data preprocessing, Illumina GenomeStudio software was used to export expression values and calculated detection p-values for each probe of each sample. Signal-intensity data were log 2 transformed and quantile-normalized. Only probes with a significant detection p-value (p<0.05) in at least one of sample were considered. The data was submitted to GEO and is accessible using accession number GSE79404. Differential expression for probes was tested using SAM (‘significance analysis of microarrays’) method⁵⁵. Multiple groups were compared using “Multiclass” option and matched patient samples groups were compared using “Two sample paired” option. False discovery rate was estimated using Storey et. al procedure⁴¹. Genes with a false-discovery rate of <5% were considered significant unless stated otherwise.

Hierarchical cluster was performed using standardized Euclidean distance with average linkage. Genes that had GO annotation GO:0005886 (plasma membrane) and either GO:0004872 (receptor activity) or GO:0009897 (external side of plasma membrane) were considered as a candidate for a surface molecule marker. For expression heatmaps samples from the same patient were additionally normalized to the average between them, and samples from healthy donors were normalized to average between all patient samples.

Enrichment analyses were done using QIAGEN's Ingenuity Pathway Analysis software (IPA®, QIAGEN Redwood City, www.qiagen.com/ingenuity). Pathway results with FDR<5% and p<10-5 were considered significant. Only regulators that passed p<10-8 threshold with significantly predicted (Z>2) activation state in PMN-MDSCs were reported.

For OLR1 gene expression association with cancer, Oncomine (https://www.oncomine.org) was used with “Cancer vs. Normal” gene report without any additional filters. Additionally, TCGA RNAseqV2 level 3 data (https://tcga-data.nci.nih.gov) was used and RPKM expression values were compared between cancer and normal tissues (where available) using t-test. Association with survival was done using univariate cox regression and Kaplan-Meier curves were plotted for patients split into two groups using median expression. Results with p<0.05 were considered significant.

qRTPCR:

Total RNA was prepared with E.Z.N.A total RNA isolation Kit I (Omega Biotek) and cDNA was synthesized with High-Capacity cDNA Reverse Transcription Kit (Applied Biosystems). Quantitative RT-PCR was performed with Power SYBR Green PCR Master Mix (Applied Biosystems). The relative amount of mRNA was estimated by the comparative threshold cycle method with GAPDH as reference gene. For the analysis of gene expression, the following primers of Table 2 were used:

TABLE 2

| | | SEQ | | SEQ |
|---|---|---|---|---|
| | | ID | | ID |
| Gene | 5′ primer | NO: | 3′ primer | NO: |
| sXBP-1 | 5′ CTGAGTCCG | 1 | 5′ AGTTGTCCAGAATG | 2 |
| | CAGCAGGTG 3′ | | CCCAACA 3′ | |
| DDIT3 | 5′ GCACCTCCCA | 3 | 5′ GTCTACTCCAAGC | 4 |
| (CHOP) | GAGCCCTCACTC | | CTTCCCCCTGCG 3′ | |
| | TCC 3′ | | | |
| ATF4 | 5′ TTCCTGAGCA | 5 | 5′ TCCAATCTGTCC | 6 |
| | GCGAGGTGTTG 3′ | | CGGAGAAGG 3′ | |
| ATF3 | 5′ TGCCTCGGAA | 7 | 5′ GCAAAATCCTCA | 8 |
| | GTGAGTGCTT 3′ | | AACACCAGTG 3′ | |
| SEC61A | 5′ GGATGTATGGG | 9 | 5′ CTCGGCCAGTG | 10 |
| | GACCCTTCT 3′ | | TTGACAGTA 3′ | |
| ARGI | 5′ CTTGTTTCGG | 11 | 5′ CACTCTATGTAT | 12 |
| | ACTTGCTCGG 3′ | | GGGGGCTTA 3′ | |
| NOS-2 | 5′ CAGCGGGAT | 13 | 5′ AGGCAAGATT | 14 |
| | GACTTTCCAA 3′ | | TGGACCTGCA 3′ | |
| GADPH | 5′ GGAGTCAAC | 15 | 5′ GGCAACAATATC | 16 |
| | GGATTTGGTCG | | CACTTTACCAGA | |
| | TA 3′ | | GT 3′ | |

Statistics: Statistical analysis was performed using a 2-tailed Student′s t-test or Mann-Whitney test and GraphPad Prism 5 software (GraphPad Software Inc.), with significance determined at p <0.05.

### Example 8: Gene Expression Profile of Human PMN-MDSC

To compare PMN-MDSC and PMN from PB of the same patients with non-small cell lung cancer (NSCLC) and head and neck cancer (HNC) we used dual-density Histopaque gradient, the standard method of isolation of PMN-MDSC²⁸. Low density PMN-MDSC are co-purified with PBMC, whereas high density PMN are collected from lower gradient⁵. As a control, PMN from healthy donors were used. Both, low-density PMN-MDSCs and high-density PMN were purified further with CD15 magnetic beads to achieve similar high purity of both cell populations (data not shown).

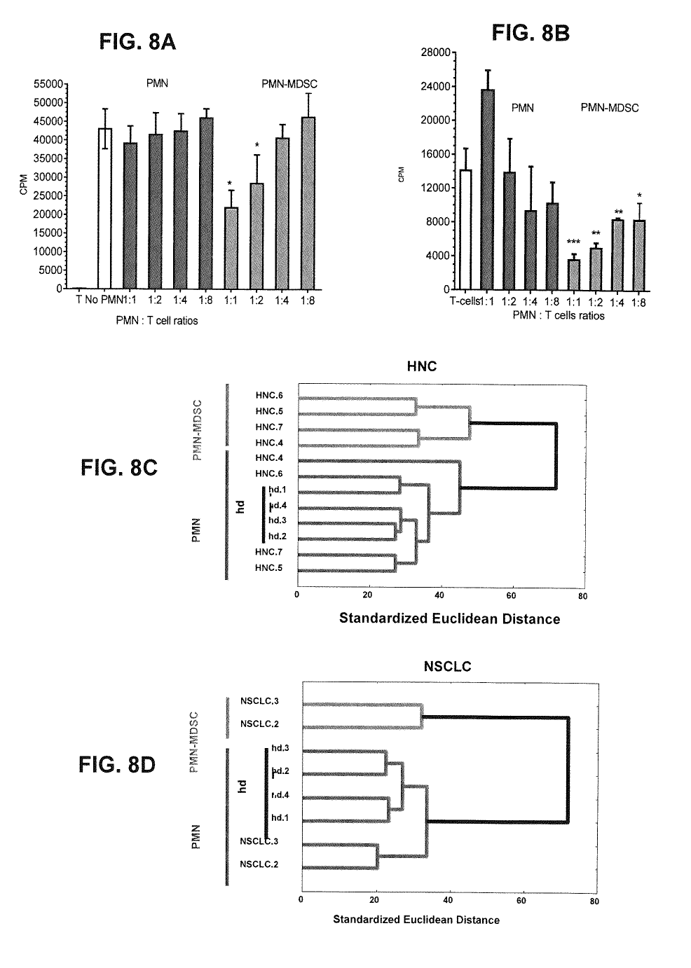

A typical phenotype of PMN-MDSC and PMN was isolated from peripheral blood of cancer patients using gradient centrifugation and CD15 beads Immune suppressive activity of PMN-MDSC, the main characteristics of these cells, was confirmed in allogeneic mixed leukocyte reaction (MLR) (FIG. 8A) and in autologous system with T cells activated by CD3/CD28 antibodies (FIG. 8B). As expected, PMN were not suppressive (FIGS. 8A and 8B)

To study overall differences and similarities between patients' PMN and PMN-MDSC as well as PMNs from healthy donors, we performed whole-genome analysis using Illumina HumanHT-12 v4 bead arrays.

A relative expression heatmap and gene/sample clustering was generated based on expression of 985 genes significantly differentially expressed (p<0.05, fold>2) between cancer patients' PMN, PMN-MDSCs and PMN of healthy donors (data not shown). Hierarchical clustering of the samples using expression of the 985 most differentially expressed genes revealed that PMN-MDSC samples have a unique expression profile and a distinct genetic signature. PMN from cancer patients are very similar to healthy donor PMN samples, as they grouped within the same cluster for HNC and NSCLC patients (FIGS. 8C and 8D).

Specifically, of the 985 genes different between any pair of groups (see Table 1), the majority (74%) showed significant differences (false discovery rate, FDR<5%) between patients' PMN-MDSC and PMN, while no genes were significantly different when corrected for multiple testing (best FDR=19%) between PMN from healthy donors and PMN from cancer patients, with only 12% of the genes significantly different at nominal p<0.05. This result indicates a high similarity of PMN samples between cancer patients and healthy donors. The direct pair-wise comparison identified 1870 array probes significantly differentially expressed (FDR<5%) between PMN-MDSC and PMN in the same patients and 36 probes showed difference of at least 5-fold. See, FIGS. 9A and 10B. See also Table 1 above for a list of 985 genes that are differentially expressed in the PMN-MCSC signature.

Using Ingenuity Pathway Analysis, we identified 14 pathways significantly enriched in PMN-MDSCs, including eukaryotic Translation Initiation Factors 2 and 4 (eIF2 and eIF4) pathways and mTOR signaling, as shown in the following Table 3.

TABLE 3

| | | | STATE IN | Z |
|---|---|---|---|---|
| CANONICAL PATHWAY | N | P | PMN-MDSC | SCORE |
| EIF2 Signaling | 54 | 4E−46 | Activated | 5.57 |
| Regulation of eIF4 and p70S6K | 26 | 7.9E−17 | | |
| Signaling | | | | |
| mTOR Signaling | 28 | 5E−16 | | 2 |
| B Cell Development | 9 | 2.1E−08 | | |
| Antigen Presentation Pathway | 9 | 6.5E−08 | | |
| Role of NFAT in Regulation | 16 | 8.9E−07 | Activated | 3 |
| of the Immune Response | | | | |
| PKCtheta Signaling in T | 13 | 1.5E−06 | Activated | 2.24 |
| Lymphocytes | | | | |
| Allograft Rejection Signaling | 11 | 1.8E−06 | | |
| T Helper Cell Differentiation | 10 | 2.6E−06 | | |
| Graft-versus-Host Disease | 8 | 7.4E−06 | | 1.63 |
| Signaling | | | | |
| CD28 Signaling in T Helper | 12 | 8.9E−06 | | |
| Cells | | | | |
| OX40 Signaling Pathway | 10 | 2.1E−05 | | |
| Autoimmune Thyroid Disease | 7 | 5.9E−05 | | |
| Signaling | | | | |
| Inhibition of Angiogenesis | 6 | 7.6E−05 | Activated | 2.24 |
| by TSP1 | | | | |

The regulators of genes enriched in PMN-MDSC included regulators of ER stress response, MAPK pathway, CSF1, IL-6, IFN-γ, NF-κB. These molecules were previously directly implicated in MDSC biology, primarily PMN-MDSC, as discussed in Reference 4, incorporated herein by reference. Surprisingly, one of the most significant changes was associated with low density lipoprotein (LDL) as shown in Table 4 below. Table 4 lists the upstream Regulators identified by Ingenuity Pathway Analysis (IPA) among genes significantly differentially expressed between PMN-MDSC and PMN cells. N=number of genes from the category, Z=z-score of predicted activation state calculated by IPA.

TABLE 4

| | | | State in | Z |
|---|---|---|---|---|
| Regulator | N | p | PMN-MDSC | score |
| MYCN | 53 | 1E−39 | Activated | 5.69 |
| MYC | 74 | 1E−22 | Activated | 4.63 |
| CSF3 | 26 | 3E−16 | Activated | 2.23 |
| IL3 | 35 | 7E−16 | Activated | 2.5 |
| TGFβ1 | 82 | 3E−15 | Activated | 2.63 |
| TNF | 81 | 1E−14 | Activated | 2.52 |
| LDL | 27 | 2E−13 | Activated | 2.91 |
| RAF1 | 25 | 8E−13 | Activated | 3.79 |
| APP | 50 | 7E−12 | Activated | 2.21 |
| IL6 | 43 | 5E−11 | Activated | 2.68 |
| PDGFBB | 27 | 3E−10 | Activated | 4.07 |
| EPO | 25 | 5E−10 | Activated | 3.08 |
| CD40LG | 29 | 7E−10 | Activated | 2.57 |
| Nek | 36 | 3E−09 | Activated | 3.61 |
| IL13 | 28 | 3E−09 | Activated | 2.01 |
| AGT | 30 | 3E−09 | Activated | 2.4 |
| IL1β | 44 | 6E−09 | Activated | 3.11 |
| ERBB2 | 37 | 6E−09 | Activated | 2.62 |
| MAP2K1 | 18 | 7E−09 | Activated | 3.05 |
| VEGFα | 21 | 2E−08 | Activated | 3.12 |
| CSF1 | 17 | 4E−08 | Activated | 2.87 |
| FLI1 | 10 | 8E−08 | Activated | 2.15 |
| Fin | 54 | 8E−08 | Activated | 3.83 |

Thus, PMN-MDSC had a distinct genomic profile from PMN isolated from the same cancer patients and PMN from healthy donors. Genes associated with ER stress response were among the most up-regulated in PMN-MDSC.

### Example 9: Lox-1 is Differentially Expressed on PMN-MDSC and PMN

To search for potential markers of PMN-MDSC we evaluated differentially expressed genes, which encoded surface molecules and compared expression of various surface molecules between PMN-MDSC and PMN from the same patients and PMN from healthy donors. More than 20 genes encoded surface molecules were found to be differentially expressed in PMNMDSC and PMN (FIG. 9A). In an attempt to validate these observations, we tested surface expression of some of the proteins using available antibodies and flow cytometry. The following proteins were found to be either expressed in large proportion of PMN or equally expressed on PMN and PMN-MDSC: CD81, CD69, CD86, integrin β5, CD42a, CD36, CD52, Annexin II (data not shown). Unexpectedly, the differences were found in the expression of lectin-like oxidized low-density lipoprotein receptor-1 (LOX-1), a 50 kDa transmembrane glycoprotein encoded by the OLR1 gene (oxidized LDL receptor 1)¹. In our analysis OLR1 was one of the mostly up-regulated genes in PMN-MDSC (FIG. S1B). LOX-1 is one of the main receptors for oxidized-LDL (oxLDL)⁴⁰. It also binds other ligands including other modified lipoproteins, advanced glycation end products, aged red blood cells, apoptotic cells and activated platelets⁴⁵ LOX-1 is expressed on endothelial cells, macrophages, smooth muscle cells, and some intestinal cell lines¹. However, it has not been associated with neutrophils or monocytes.

We evaluated LOX-1 expression in high density PMN and low-density PMN-MDSC in cancer patients. LOX-1 was practically undetectable in PMN but expressed in about ⅓ of PMN-MDSC fraction (FIG. 9B). Since LOX-1 can be expressed on platelets⁵¹ and it is known that platelets can adhere to activated PMN we asked whether increased expression of LOX-1 in PMN-MDSC fraction was the result of increased adherence of platelets. However, LOX-1- and LOX-1+ cells in low density PMN-MDSC population had the same small proportion of cells that express platelets markers CD41a and CD42b (FIG. 9C).

These results suggested that LOX-1 could be associated with PMN-MDSC. We asked whether LOX-1 can be a marker of PMN-MDSC. To test this hypothesis, it was important to avoid the use of gradient centrifugation and labeled cells in PB directly with granulocyte-specific CD15 antibody and evaluated expression of LOX-1 among all CD15+ cells. In preliminary experiments, we found no differences in the results obtained with CD15 or CD66b antibodies. We referred to CD15+ cells as PMN since Siglec-8+ eosinophils represented very small proportion of CD15+ cells and no differences in the presence of eosinophils between CD15+LOX-1+ and CD15+LOX-1− cells was seen (data not shown). The proportion of LOX-1+ cells among all PMN in healthy donors was very low (range 0.1-1.5, mean 0.7%). In patients with NSCLC it increased to 4.9% (p<0.001), in patients with HNC to 6.4% (p<0.0001), and in patients with colon cancer (CC) to 6.5% (p=0.0035) (FIGS. 9D, 9E and 9F). In all three types of cancer >75% of patients had proportion of LOX-1+ PMN higher than the range established for healthy donors.

We also assessed the changes in LOX-1+ PMN in tumor-free patients with inflammatory conditions: eosinophilic esophagitis, ulcerative colitis and Crohn's disease. Only patients with Crohn's disease had a small increase in the proportion of these cells (FIG. 9G). Thus, LOX-1 expression defined distinct population of neutrophils in cancer patients and was associated with accumulation of PMN-MDSC.

### Example 10—Lox-1 Defines the Population of PMN-MDSC Among Neutrophils

Next, we addressed the question whether LOX-1 can be considered as marker of human PMN-MDSC. LOX-1+ and LOX-1− PMN were sorted directly from PB of the same patients. LOX-1− PMN had the typical morphology of mature neutrophils, whereas LOX-1+ PMN displayed more immature morphology with band shape nuclei (data not shown). Whole genome array was performed on LOX-1+ and LOX-1− PMN and compared with that of PMN and PMN-MDSC. Analysis of gene expression revealed 639 genes significantly different between LOX-1+ and LOX-1− (FDR<5%, fold>2) and based on expression of those genes LOX-1+ PMN clustered together with PMN-MDSC, whereas LOX-1− PMN were very similar to patients' and healthy donor's PMN (FIG. 10A). Overall, 92% of those genes had the same direction of change between LOX1+/LOX1− as between PMN-MDSC/PMN with 93 probes significantly upregulated (FDR<5%) at least 2 fold or more in both PMN-MDSC and LOX-1+ PMN (FIG. 10B and FIG. S2). Thus, LOX-1+ PMN from cancer patients had genomic profile similar to that of PMN-MDSC.

The hallmark of PMN-MDSC is their ability to suppress T-cell function. We isolated LOX-1- and LOX-1+ PMN directly from PB of cancer patients and used them in T-cell suppression assay. LOX-1+ PMN suppressed T-cell proliferation, whereas LOX-1− PMN did not (FIG. 10C). We asked whether the LOX-1 antibody used for isolation of PMN-MDSC could directly affect the functional activity of PMN. PMN isolated from cancer patients were cultured with T cells in the presence of LOX-1 antibody or IgG isotype control. LOX-1 antibody did not make PMN acquire immune suppressive function (FIG. S3).

We then evaluated possible mechanisms responsible for LOX-1+ PMN-MDSC suppression. We tested several common mechanisms implicated in PMN-MDSC function. LOX-1+ PMN-MDSC had significantly higher production of reactive oxygen species (ROS) than LOX-1− PMN (FIG. 10D). Whole genome array showed that LOX-1+ PMN-MDSC had higher expression of ARG1, the gene directly associated with PMN-MDSC function, than LOX-1− PMN. These differences were not statistically significant (FDR=7%). However, direct evaluation of ARG1 expression by qPCR revealed significantly higher expression of this gene in LOX-1+ PMN-MDSC than in LOX-1− PMN (FIGS. 10E and 10F). Expression of NOS2 in PMN was much lower than that of ARG1. However, it was still significantly higher in LOX-1+ PMN-MDSC than in LOX-1− PMN (FIGS. 10E and 10F). We tested the contribution of ROS and ARG1 to immune suppression mediated by LOX-1+ PMN-MDSC. Both, ROS scavenger N-acetylcysteine (NAC) and catalase substantially decreased LOX-expression in PMN obtained from healthy donors (FIGS. 10G and 10H) as well as inhibitor of Arg1 Nor-NOHA (FIG. 10I). Thus, taken together these data demonstrate that LOX-1+ PMN indeed represent a population of PMN-MDSC.

Then, we investigated the possible role of LOX-1 as marker of mouse PMN-MDSC. Similar to human PMN, CD11b+Ly6CloLy6G+ mouse PMN had very low expression of LOX-1. However, in contrast to human PMN-MDSC, spleen, BM, or tumor PMN-MDSC from mice bearing EL-4 lymphoma, Lewis Lung Carcinoma (LLC) or transgenic Ret melanoma did not up-regulate LOX-1 (data not shown). To evaluate the possible role of LOX-1 in PMN-MDSC function we used bone marrow (BM) cells from LOX-1 knockout (olr1−/) mice³¹. Lethally irradiated wildtype recipients were reconstituted with congenic bone marrow cells isolated from wild-type or olr1−/− mice. Ten weeks after reconstitution donor's cells represented more than 95% of all myeloid cells. LLC tumor was implanted s.c. and mice evaluated 3 weeks later. No differences in the presence of PMN-MDSC in spleens or tumors were observed between mice reconstituted with WT and LOX-1 KO BM (data not shown). WT and olr1−/− PMN-MDSC suppressed T-cell proliferation equally well (data not shown). Gene expression profile demonstrated no differences between WT and olr1−/− PMN-MDSC. Most importantly WT PMN-MDSC had the same undetectable level of olr1 expression as olr1−/− PMN-MDSC (data not shown). Thus, in contrast to humans, mouse LOX-1 is not associated with PMN-MDSC.

### Example 11: Mechanism Regulating LOX-1 Expression in PMN-MDSC

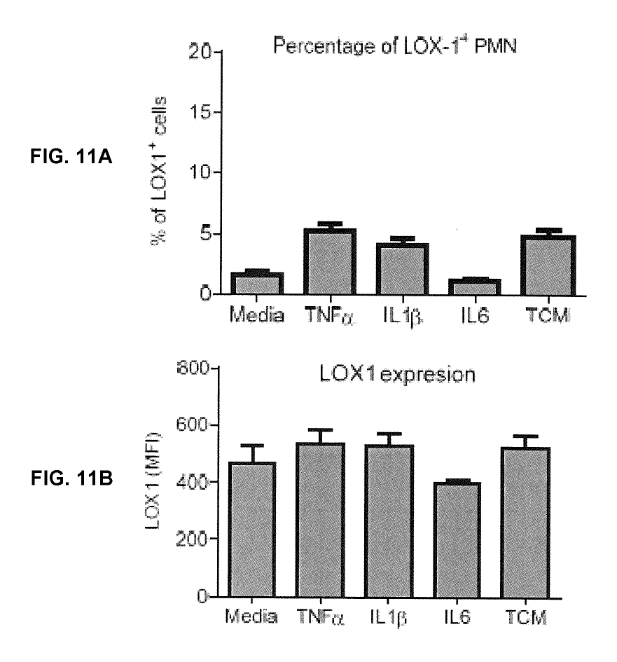

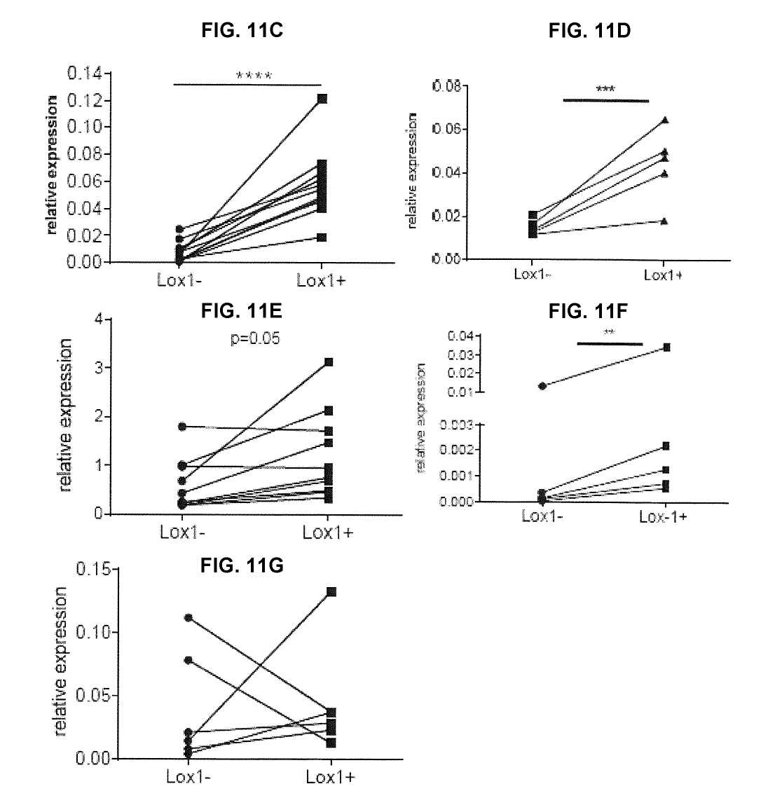

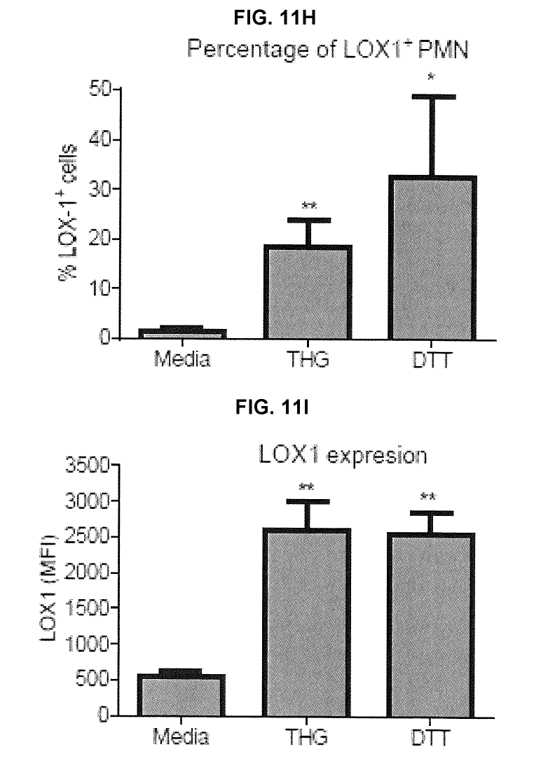

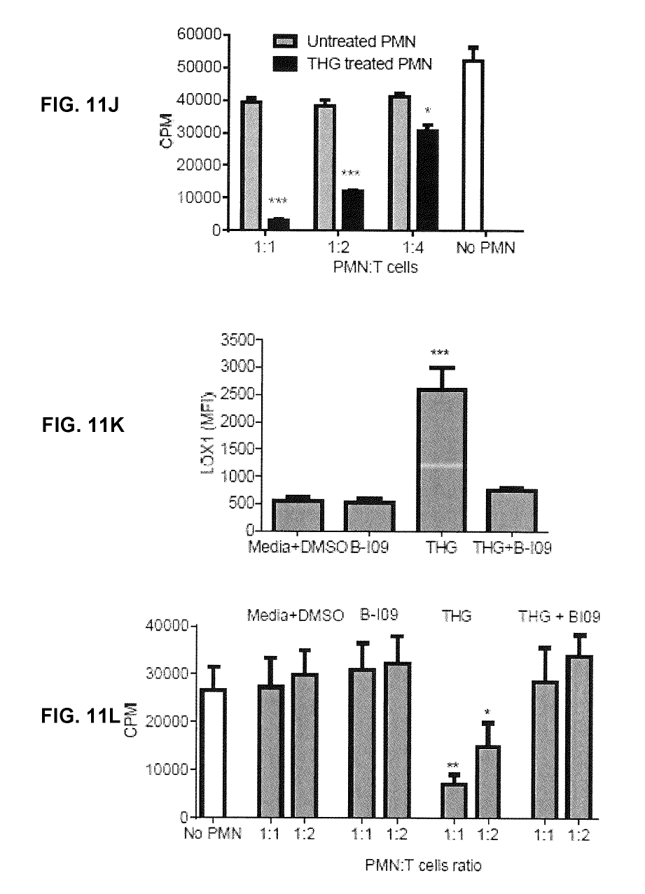

What could induce LOX-1 up-regulation in PMN-MDSC? Based on the fact that in endothelial cells LOX-1 can be induced by pro-inflammatory cytokines³⁵, we tested the effect of several cytokines as well as tumor-cell conditioned medium (TCM) on LOX-1 expression in PMN isolated from healthy donors. None of the tested pro-inflammatory cytokines (IL-1β, TNFα, IL-6) or TCM induced upregulation of LOX-1 in PMN after 24 hr culture (GM-CSF was added to protect PMN viability) (FIGS. 11A and 11B).

Our previous observations⁵ and data obtained in this study demonstrated that PMN-MDSC in cancer patients displayed signs of ER stress response. LOX-1+ and LOX-1⁻ PMN were isolated from PB of cancer patients and expression of genes associated with ER-stress were evaluated. LOX-1+ PMN-MDSC had significantly (p<0.001) higher expression of sXBP1 (FIG. 11C) and its target gene SEC61a (FIG. 11D) than LOX-1-PMN. Expression of ATF4 (FIG. 11E) and its target gene ATF3 (FIG. 11F) was also significantly (p<0.05) higher in LOX-1+ PMN-MDSC. No changes in the expression of CHOP were observed (FIG. 11G). To test the effect of ER-stress on expression of LOX-1, PMN from healthy donors were treated with ER stress inducers: thapsigargin (THG) or dithiothreitol (DTT) overnight in the presence of GM-CSF. At selected doses (THG—1 DTT—1 mM) cell viability remained above 95%. Both, THG and DTT caused dramatic up-regulation of LOX-1 expression in PMN (FIGS. 11H and 11I).

Overnight THG treatment of PMN also caused acquisition of potent immune suppressive activity by the PMN (FIG. 11J). Since LOX-1+ PMN-MDSC have increased expression preferentially of one of ER stress sensors—sXBP1, we verified the role of ER stress using a recently developed selective inhibitor of sXBP1—B409⁴³. In the presence of B-I09 THG failed to induce up-regulation of LOX-1 (FIG. 11K) and immune suppressive activity of PMN (FIG. 11L). Thus, induction of ER stress in control neutrophils converted these cells to immune suppressive PMN-MDSC, which was associated with up-regulation of LOX-1 expression.

### Example 12: LOX-1 in Tumor PMN-MDSC

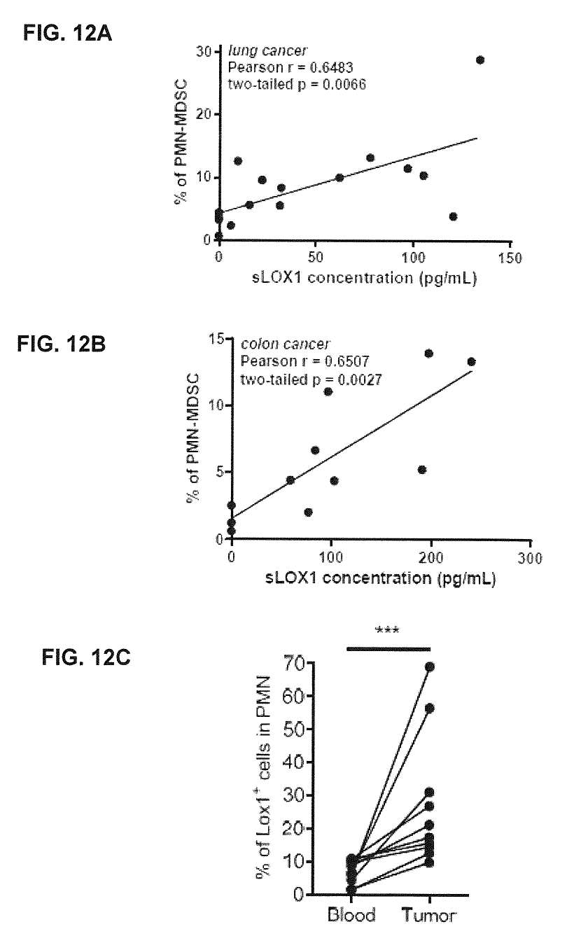

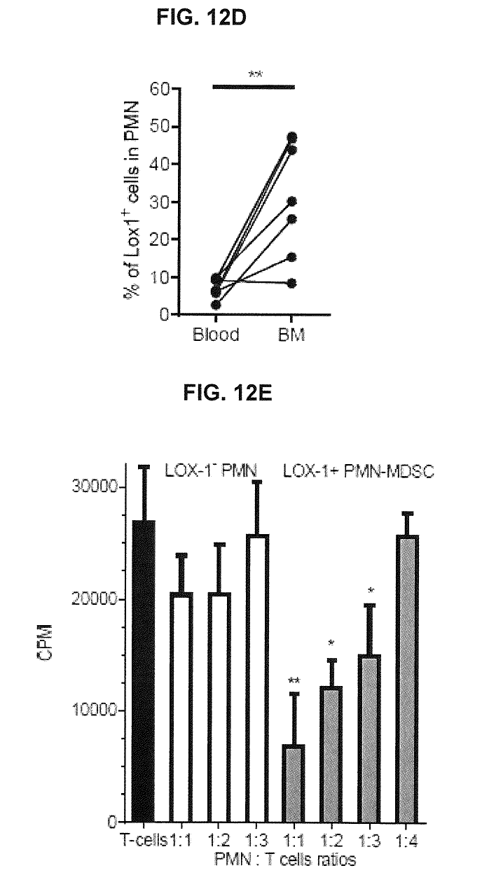

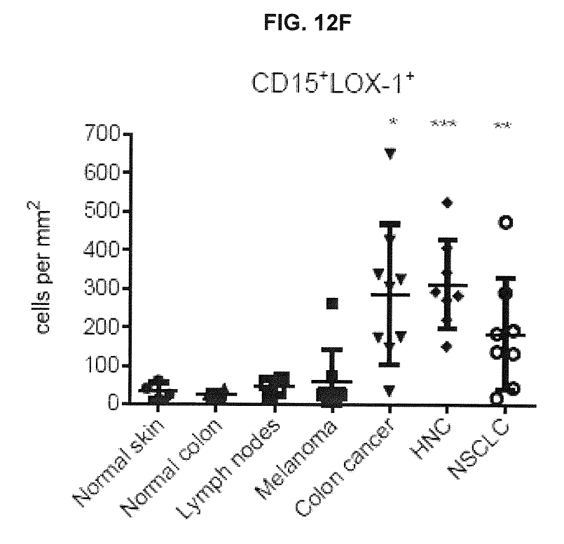

It is known that LOX-1 is shed from the surface of the cells and can be detected in plasma³⁹. We evaluated correlation between the presence of PMN-MDSC in cancer patients and soluble LOX-1 in plasma. In NSCLC and CC patients, the proportion of PMN-MDSC strongly correlated with soluble LOX-1 (FIGS. 12A and 12B) suggesting that these cells may be an important source of LOX-1 in plasma of cancer patients.

There is now sufficient evidence demonstrating that tumor MDSC are more suppressive than cells in PB²¹. We asked whether the population of PMN-MDSC is more prevalent among all PMN in tumors than in PB. The proportion of LOX-1+ cells in CD15+ PMN isolated from tumors of patients with HNC and NSCLC was >3-fold higher than in CD15+ PMN from PB of the same patients (p<0.001) (FIG. 12C). Cells in blood and tumor tissues were subjected to the same digestion protocol. However, to exclude possible effect of tissue digestions and isolation on LOX-1 expression, we also evaluated patients with multiple myeloma (MM) where the tumor is located in bone marrow (BM). We previously have shown substantial increase of PMN-MDSC in both BM and PB of MM patients³⁷. Similar to solid tumors, the proportion of LOX-1+ PMN-MDSC in BM was 3-4-fold higher than in PB of the same patients (p=0.004) (FIG. 12D). LOX-1+ PMN-MDSC isolated from BM of patients with MM had profound immune suppressive activity, whereas LOX-1− PMN did not suppress T cells (FIG. 12E) supporting the conclusion that LOX-1+ PMN represent PMN-MDSC at the tumor site.

To evaluate the presence of LOX-1+ PMN-MDSC in tumor tissues, we have developed a method of immune fluorescent staining of paraffin-embedded tissues with combination of LOX-1 and CD15 antibody (data not shown). Control tissues from normal skin, colon and lymph nodes had similar low numbers of LOX-1+CD15+ PMN-MDSC (FIG. 12F). No statistical differences were found in the presence of these cells in melanoma samples, which is consistent with findings that MMDSC but not PMN-MDSC are the predominant population of MDSC in these patients³². The number of LOX-1+CD15+ PMN-MDSC in colon carcinoma increased more than 8-fold, in HNC more than 10-fold, and in NSCLC almost 8-fold (FIG. 12F). Thus, LOX-1 expression defines the population of PMN-MDSC in tumor tissues.

### Example 13: Association of OLR-1 Expression and the Presence of LOX-1+ PMN-MDSC with Clinical Parameters

Using ONCOMINE and TCGA databases we evaluated the association of OLR1 expression in tumor tissues with clinical parameters in different types of cancer. Significant upregulation of OLR1 was observed in many types of cancer. As shown in Table 5, there is a clinical association of OLR1 expression and LOX-1+ PMN-MDSC accumulation in cancer patients. The following Table 5 shows the number of independent data sets from Oncomine database that showed OLR1 upregulated (up) or downregulated (down) in Cancer vs Normal tissues. P-value and fold change for Cancer/Normal comparison from TCGA database. na=data not available;

TABLE 5
Expression of OLR1 in different types of
cancer

| | TCGA | Oncomine | | TCGA | |
|---|---|---|---|---|---|
| Description | code | up | down | pv | fold |
| Bladder | BLCA | | | 0.0014 | 4.06 |
| Urothelial | | | | | |
| Carcinoma | | | | | |
| Breast invasive | BRCA | 13 | | 1E-64 | 5.22 |
| carcinoma | | | | | |
| Cervical and | CESC | 1 | | 0.0026 | 13.74 |
| endocervical | | | | | |
| carcinoma | | | | | |
| Colon | COAD | 5 | | 2E-18 | 7.12 |
| adenocarcinoma | | | | | |
| Glioblastoma | GBM | | | 0.5334 | 1.29 |
| multiforme | | | | | |
| Head and Neck | HNSC | 2 | 1 | 2E-07 | 2.96 |
| squamous cell | | | | | |
| carcinoma | | | | | |
| Kidney | KICH | 1 | 1 | 0.437 | 1.28 |
| Chromophobe | | | | | |
| Kidney renal | KIRC | 1 | | 5E-13 | 2.73 |
| clear cell | | | | | |
| carcinoma | | | | | |
| Kidney renal | KIRP | 1 | | 0.0002 | 2.4 |
| papillary | | | | | |
| cell carcinoma | | | | | |
| Liver | LIHC | | | 0.661 | 1.11 |
| hepatocellular | | | | | |
| carcinoma | | | | | |
| Lung | LUAD | | | 6E-42 | −8.08 |
| adenocarcinoma | | | | | |
| Lung squamous | LUSC | | | 1E-54 | 16.88 |
| cell carcinoma | | | | | |
| Pancreatic | PAAD | 1 | | 0.7953 | −1.17 |
| adenocarcinoma | | | | | |
| Prostate | PRAD | | | 8E-05 | 1.67 |
| adenocarcinoma | | | | | |
| Rectum | READ | | | 9E-05 | 6.7 |
| adenocarcinoma | | | | | |
| Sarcoma | SARC | | | 0.3766 | -2.98 |
| Skin Cutaneous | SKCM | | | 0.6397 | -2 |
| Melanoma | | | | | |
| Leukemia | na | | 6 | na | na |
| Gastric | na | 1 | | na | na |
| Brain | na | 1 | | na | na |
| All cancers | All | na | na | 7E-06 | 1.39 |
| combined | cancers | | | | |

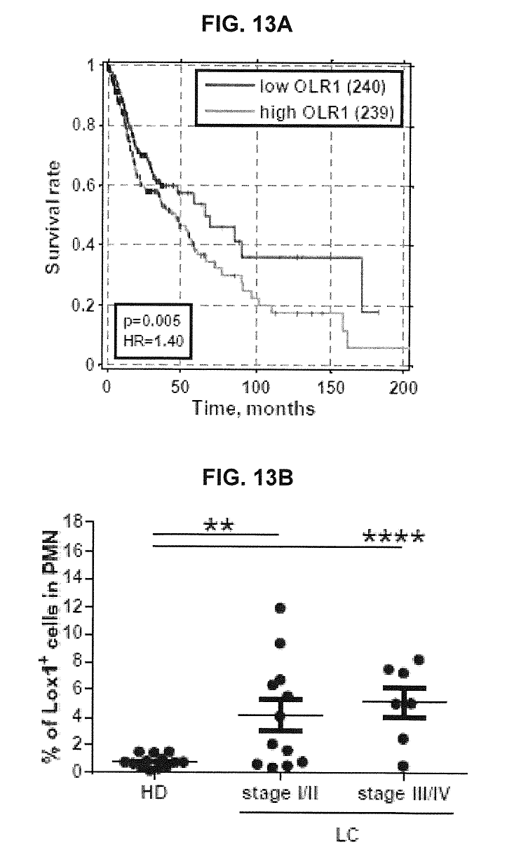

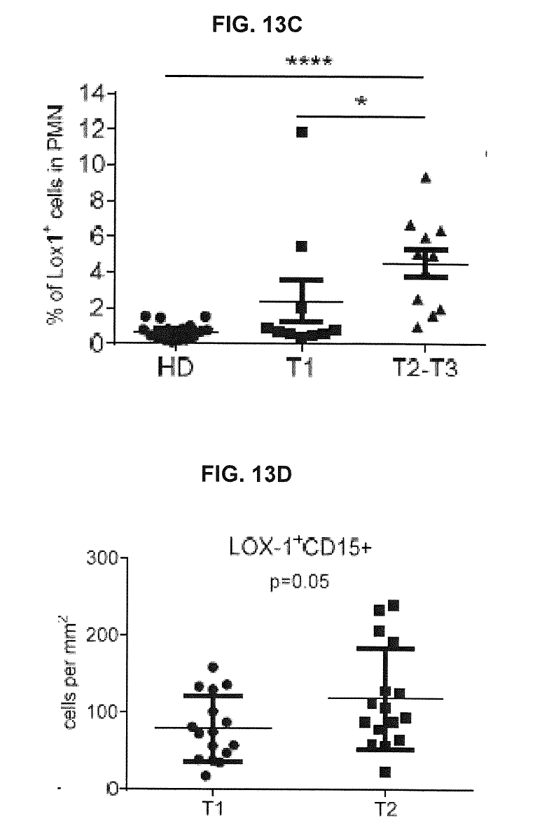

The notable exception was lung cancer, where normal lung tissues showed dramatically higher expression of OLR1 than other normal tissues, apparently due to cells with high expression of OLR1 (possibly lung epithelium). OLR1 expression positively correlated with clinical stage in patients with bladder cancer, colon adenocarcinoma, and clear cell kidney cancer. The positive correlation with tumor size was found in patients with prostate adenocarcinoma and rectal adenocarcinoma (data not shown). Higher expression of OLR1 was associated with worse survival in patients with HNC (FIG. 13A).

Although these results are suggestive, their interpretation as reflecting PMN-MDSC presence has some limitation due to the fact that OLR1 can be expressed on different cells in the tumor microenvironment. We focused on evaluation of LOX-1+ PMN-MDSC in tumor tissues and PB.

In patients with NSCLC we evaluated the possible link between stage of the disease and the proportion of LOX-1+ PMN-MDSC in PB. Patients with both early (I/II) and late (III/IV) stages of NSCLC had significantly higher proportion of LOX-1+ PMN-MDSC than in healthy donors (p<0.01 and p<0.0001 respectively). There was no statistical significant difference between these two groups of patients (FIG. 13B). However, whereas 85.7% of all patients with late stages of NSCLC had an increase in LOX-1+ PMN-MDSC population, only 50% of patients with early stages showed elevated level of these cells (FIG. 13B).

Significant association of the presence of LOX-1+ PMN in PB of cancer patients was found with size of the tumors. Only patients with large tumors (T2-T3) had significantly (p<0.001) higher proportion of LOX-1+ PMN than healthy donors, whereas patients with small tumors (T1) had similar very low level of LOX-1+ PMN as healthy donors. Patients with large tumors had significantly more LOX-1+ PMN (p<0.05) than patients with small tumors (FIG. 13C). Using a NSCLC adenocarcinoma tissue array, we evaluated the association between the presence of LOX-1+ PMN in tumor tissues and tumor size. Similar to the data obtained in PB no significant association was found with stage of the disease. However, the number of LOX-1+ PMN-MDSC was significantly higher in larger (T2 vs. T1) tumors (FIG. 13D).

As revealed by these examples, PMN-MDSC have a unique gene expression profile, which is substantially different from that of PMN from the same patients and from healthy donors. This directly supports that PMN-MDSC represent a distinct functional state of pathological activation of neutrophils in cancer^{15,29} and is consistent with the analysis of gene expression performed in mice, which demonstrated differences in transcriptome between granulocytes isolated from naïve mice and PMN-MDSC from tumor-bearing mice¹³.

Up-regulation of genes associated with ER stress response was one of the most prominent features of PMN-MDSC. The ER stress response is developed to protect cells from various stress conditions including hypoxia, nutrient deprivation, low pH, etc. and includes three major signaling cascades initiated by three protein sensors: PERK (protein kinase RNA (PKR)-like ER kinase), IRE-1 (inositol-requiring enzyme 1) and ATF6 (activating transcription factor 6)¹⁷. PERK phosphorylates eukaryotic protein synthesis initiation factor 2 alpha (eIF2α), which controls the initiation of mRNA translation and inhibits the flux of synthesized proteins. eIF2α induces the expression of ATF4 and its downstream targets, including the pro-apoptotic transcription factor CHOP. IRE1 cleaves the mRNA encoding for the transcription factor X-box binding protein-1 (XBP1)³⁸. Spliced XBP1 (sXBP1) mRNA is then ligated by a RNA ligase and translated to produce sXBP1 transcription factor that regulates many target genes including SEC61a³.

Factors implicated in LOX-1 up-regulation include Angiotensin II (Ang II), C-reactive protein (CRP), Endothelin-1 (ET-1), Glucose, Histamine, Homocysteine, Human cytomegalovirus (HCMV), Interferon-γ (IFN-γ), Interleukin-1β (IL-1β), Oxidant species, Oxidized-low density lipoprotein (ox-LDL), Phorbol ester, Shear stress, Transforming growth factor-β (TGF-β) and Tumor necrosis factor-α (TNF-α).

ER stress response was previously shown to be transmitted to dendritic cells and macrophages from tumor cells and was associated with up-regulation of arginase-1 in macrophages^{25,26,27}. Constitutive activation of XBP1 in tumor-associated dendritic cells promoted ovarian cancer progression by blunting anti-tumor immunity⁴⁸. We have recently found activation of ER stress response in MDSC⁵. We demonstrated that MDSC isolated from tumor-bearing mice or cancer patients overexpressed sXBP1 and CHOP, and displayed an enlarged endoplasmic reticulum, one of the hallmarks of the ER stress⁵. Other study implicated CHOP in the suppressive activity of MDSC in tumor site⁴⁶. Consistent with these observations administration of an ER stress inducer to tumor-bearing mice increased the accumulation of MDSC and their suppressive activity²².

We have discovered that expression of LOX-1 receptor was associated with PMN-MDSC. LOX-1 is a class E scavenger receptor expressed on macrophages and chondrocytes, as well as endothelial and smooth muscle cells⁴⁵. Expression of this receptor on neutrophils previously was not described. We have found that neutrophils from healthy donors and cancer patients have practically undetectable expression of LOX-1. Our data indicated that LOX-1 expression is not just associated with, but actually defines, the population of PMN-MDSC in cancer patients. This is supported by several lines of evidence. LOX-1+ PMN had a gene expression profile similar to that of enriched PMN-MDSC isolated using gradient centrifugation.

In contrast, LOX-1⁻ PMN had a profile similar to neutrophils. LOX-1⁺ but not LOX-1⁻ PMN potently suppressed T-cell response. Finally, LOX-1+ PMN had significantly higher expression of ARG1 and production of ROS, typical characteristics of PMN-MDSC. We found that in tumor tissues, only LOX-1+ PMN were immune suppressive and could be considered as PMN-MDSC. This permits a direct identification of PMN-MDSC in PB and tumor tissues.

These observations, although unexpected, fit the overall concept of a critical role of ER stress response in MDSC biology. It was recently demonstrated that in human endothelial cells oxLDL induced expression of LOX-1 through activation of ER stress sensors IRE1 and PERK¹⁹. Ox-LDL induces LOX-1-dependent ER stress¹⁹. In contrast, ER stress induced by tunicamycin in hepatic L02 cells caused down-regulation of LOX-1. Knock down of IRE1 or XBP-1 restored LOX-1 expression in these cells¹⁸.

It is likely that signaling through LOX-1 is responsible for, or at least contributes to, acquisition of immune suppressive activity by neutrophils. Engagement of LOX-1 can lead to induction of oxidative stress, apoptosis, and activation of the NF-κB pathway¹. These pathways are known to be important for PMN-MDSC function. The ER stress response pathway has been shown to regulate inflammation by activating the NF-κB pathway^{3,2,54} LOX-1 up-regulation has been observed during cellular transformation into cancer cell and can have a pro-oncogenic effect by activating the NF-κB pathway, by increasing DNA damage through increase ROS production and by promoting angiogenesis and cell dissemination^{16,24}. It is possible that LOX-1 signaling may drive pathological activation of PMN towards PMN-MDSC. Cell surface LOX-1 expression can be elevated by multiple stimuli including reactive oxygen species (ROS), inflammatory cytokines (TNF-α, TGF-β) as well as oxLDL⁴⁹. These factors are produced in cancer and it is possible that they can affect differentiation of granulocytes from precursors leading to acquisition of LOX-1 expression.

Our data demonstrated that patients have variable amount of LOX-1+ PMN-MDSC, which at least in patients with NSCLC was associated with size of the tumors. It is theorized that the presence of these cells in tumor tissues can predict clinical outcome. Expression of LOX-1 on PMN-MDSC opens an opportunity for selective targeting of these cells, since antibody targeting LOX-1 have been already tested in cardiovascular diseases in mice^{7,30}. Average expression values (FPKM values) for cancer and normal tissues for different cancers indicates high baseline expression level of OLR1 in normal lung tissues, as shown in Table 6.

TABLE 6

| TGCA | Expression | | |
|---|---|---|---|
| code | Cancer | Normal | Type |
| BLCA | 139.1 | 34.3 | Bladder Urothelial Carcinoma |
| BRCA | 288.8 | 55.3 | Breast invasive carcinoma |
| CESC | 111.6 | 8.1 | Cervical and endocervical |
| | | | carcinoma |
| COAD | 57.8 | 8.1 | Colon adenocarcinoma |
| GBM | 591.4 | 459.5 | Glioblastoma multiforme |
| HNSC | 86.5 | 29.3 | Head and Neck squamous cell |
| | | | carcinoma |
| KICH | 99.0 | 77.1 | Kidney Chromophobe |
| KIRC | 306.4 | 112.1 | Kidney renal clear cell |
| | | | carcinoma |
| KIRP | 311.5 | 129.9 | Kidney renal papillary cell |
| | | | carcinoma |
| LIHC | 12.4 | 11.2 | Liver hepatocellular carcinoma |
| LUAD | 408.8 | 3301.5 | Lung adenocarcinoma |
| LUSC | 199.7 | 3370.8 | Lung squamous cell carcinoma |
| PAAD | 497.6 | 582.7 | Pancreatic adenocarcinoma |
| PRAD | 47.3 | 28.3 | Prostate adenocarcinoma |
| READ | 49.1 | 7.3 | Rectum adenocarcinoma |
| SARC | 16.9 | 50.4 | Sarcoma |
| SKCM | 41.1 | 82.3 | Skin Cutaneous Melanoma |

Specific embodiments of the methods and compositions described herein include:

A method for monitoring the population of polymorphonuclear myeloid derived suppressor cells (PMN-MDSCs) in a mammalian subject comprising: contacting a biological sample from the subject containing polymorphonuclear neutrophils (PMNs) and PMN-MDSC with a ligand that specifically binds or forms a complex with LOX-1on the cell surface; and detecting and distinguishing the complexes of ligand-bound LOX-1-cells from other cells not bound to the ligand in the sample, wherein the LOX-1-bound cells are PMN-MDSCs substantially free of PMN. Also included in an embodiment of the method further comprising counting the cells bound to the ligand to obtain a LOX-1+ population. Also included in an embodiment of the method wherein said ligand is an anti-LOX-1 antibody, an anti-LOX-1 antibody fragment, optionally associated with a detectable label component. Also included in an embodiment of the method further comprising contacting the sample with a ligand that specifically binds or forms a complex with a neutrophil biomarker to identify PMN in the sample.

In certain embodiments of these methods, the neutrophil biomarker is CD15 or CD66b. In certain embodiments of these methods, the ligand is an anti-CD15 antibody, an anti-CD15 antibody fragment, optionally associated with a detectable label component. In other embodiment of these methods, the ligand is an anti-CD66b antibody, an anti-CD66b antibody fragment, optionally associated with a detectable label component.

Another embodiment of the method employs the ligand immobilized on a substrate or associated with a detectable label component. In such embodiments, the detectable label component is independently detectable or is capable of generating a measurable detectable signal when contacted with another label component. In certain embodiments, the separating step comprises washing the unbound cells and other debris in the sample from the substrate and counting or collecting the bound PMN-MDSCs from the substrate. In other embodiments, the separating step comprises treating the sample with a reagent which identifies LOX-1-PMN-MDSC complexes from unbound cells to permit enumeration of PMN-MCSC. Other specific embodiments comprise separating bound cells from unbound cells in the sample based on size exclusion.

In still other embodiments, the method further comprises a step of contacting the sample with biomarkers that identify as a single population both PMN-MDSCs and PMNs and isolating a cell suspension containing both PMN-MDSCs and PMNs prior to contacting the cell suspension with the LOX-1 ligand.

In other specific embodiments of these methods, the biological sample is whole blood and the method further comprises destroying or lysing any red blood cells in the sample. In yet other embodiments, the methods involve collecting a second population which is not immobilized on the substrate, this second population containing PMNs and being substantially free from PMN-MDSCs.

Another specific embodiment is a method of differentiating polymorphonuclear myeloid derived suppressor cells (PMN-MDSCs) from polymorphonuclear neutrophils (PMNs) in a biological sample containing both types of cells comprising: contacting the sample with a ligand that specifically binds or forms a complex with LOX-1on the cell surface; and detecting and separating the complexes of ligand-bound LOX-1-cells from other cells not bound to the ligand in the sample, wherein the LOX-1-bound cells are PMN-MDSCs substantially free of PMN.

Still another specific method is designed for obtaining a population of cells enriched in human polymorphonuclear myeloid derived suppressor cells (PMN-MDSCs) and comprises isolating from a cell suspension those cells which express LOX-1 to provide a population of cells enriched with PMN-MDSCs. In certain embodiment, these methods also comprise detecting a population of LOX-1⁺ cells greater than 1% of the total neutrophil population in the sample of a subject, wherein said population of LOX-1+ cells indicates the presence, progression or metastasis of a cancer. Thus, other embodiments involve measuring the concentration of soluble LOX-1 in the serum of the subject and correlating that concentration with the concentration of PMN-MDSC in the subject.

Other embodiments involve a composition comprising a ligand that specifically binds or forms a complex with LOX-1 on the cell surface for use in detecting or obtaining a population of human polymorphonuclear myeloid derived suppressor cells (PMN-MDSCs) and diagnosing the presence, progression or metastasis of a cancer.

In still other embodiments, any of these methods can further comprise contacting the sample with a reagent that identifies activators or regulators of ER stress response in said cells or a reagent that identifies other biomarkers that distinguish PMN-MDSC from PMN. In certain embodiment, the activators are one or more of sXBP1, DDIT3 (CHOP), ATF4, ATF3, SEC61A ARGI or NOS-2. In other embodiments, the regulators or biomarkers are one or more of one or more of MYCN, CSF3, IL3, TGFβ1, TNF, LDL, RAF1, APP, IL6 PDGFBB, EPO, CD40LG, Nek, IL13, AGT, IL1β, ERBB2, MAP2K1, VEGFα, CSF1, FLI1, or Fin or the biomarkers of Table 1, FIG. 9A or FIG. 10B.

In still other specific embodiments, a composition comprises a ligand that specifically binds or forms a complex with LOX-1 on the cell surface for use in detecting or obtaining a population of human polymorphonuclear myeloid derived suppressor cells (PMN-MDSCs) and diagnosing the presence, progression or metastasis of a cancer.

In yet other embodiments, a pharmaceutical composition that reduces or inhibits ER stress in mammalian neutrophils or reduces or inhibits LOX-1 expression on neutrophil populations in a pharmaceutically acceptable carrier or excipient is provided. In some embodiment, such a composition comprises an antagonist or inhibitor of the expression, activity or activation of one or more of sXBP1, DDIT3 (CHOP), ATF4, ATF3, SEC61A ARGI or NOS-2. In other embodiments, such a composition contains an antagonist or inhibitor of LOX-1. In other embodiments, such a composition contains an antagonist or inhibitor of the expression, activity or activation of one or more of MYCN, CSF3, IL3, TGFβ1, TNF, LDL, RAF1, APP, IL6 PDGFBB, EPO, CD40LG, Nek, IL13, AGT, IL1β, ERBB2, MAP2K1, VEGFα, CSF1, FLI1, or Fin. In any of these compositions, the antagonist or inhibitor is an antibody, functional antibody fragment, single chain antibody, or equivalent.

In yet another specific embodiment a method for reducing or inhibiting LOX-1+ PMN-MDSC accumulation in a cancer patient comprises administering a composition as described herein.

In another specific embodiment, a method of diagnosing a mammalian subject with a cancer comprises obtaining a biological sample from the subject; detecting whether soluble LOX-1 is present in the sample by contacting the sample with an antibody or functional antibody fragments that specifically binds or forms a complex with LOX-1 on the cell surface; detecting and distinguishing the complexes of antibody-bound LOX-1− cells from other cells not bound to the antibody in the sample, and correlating the size of a tumor in the subject with the number of LOX-1+ PMN or PMN-MDSC detected.

Yet another method of treating a cancer comprises obtaining a biological sample from a subject; detecting whether PMN-MDSC are present in the sample; diagnosing the subject with cancer when the presence of LOX-1+ is detected at a level that indicates PMN-MDSC are present; and administering an effective amount of a composition that reduces or inhibits ER stress response in mammalian LOX-1+ neutrophils, LOX-1+ PNM or PMN-MDSC or reduces or inhibits LOX-1 expression on LOX-1+ neutrophils, LOX-1+ PNM or PMN-MDSC.

Yet another method of treating a cancer comprises the use of immunotherapeutics. The patient having cancer is administered an antibody or functional antigen-binding fragment that binds to LOX-1. In another embodiment, a method of treating cancer involves administering an antibody or functional antigen-binding fragment that inhibits that inhibits the expression, activity or activation of at least one of sXBP1, DDIT3 (CHOP), ATF4, ATF3, SEC61A ARGI, MYCN, CSF3, IL3, TGFβ1, TNF, LDL, RAF1, APP, IL6 PDGFBB, EPO, CD40LG, Nek, IL13, AGT, IL1β, ERBB2, MAP2K1, VEGFα, CSF1, FLI1, or Fin. In another embodiment, the method involves inhibiting the expression, activity or activation of one or more of the biomarkers of FIG. 9A, FIG. 10B or Table 1, or CD15 or CD66b. In still other embodiments, this immunotherapeutic step is combined with the diagnostic methods described above.

Each and every patent, patent application and any document listed herein, particularly references 4, 6, 7, 21, 28 and 29, and the sequence of any publically available nucleic acid and/or peptide sequence cited throughout the disclosure, is/are expressly incorporated herein by reference in its entirety. Embodiments and variations of this invention other than those specifically disclosed above may be devised by others skilled in the art without departing from the true spirit and scope of the invention. The appended claims include such embodiments and equivalent variations.

### REFERENCES

1. Al-Banna, N. et al. 2013, Oxidized LDL and LOX-1 in experimental sepsis. Mediators of Inflammation, 76: 178-9.
2. Bettigole, S E et al. December 2014, Endoplasmic Reticulum Stress in Immunity. Annu Rev Immunol, 33:107-38
3. Claudio, N et al, May 2013, Mapping the crossroads of immune activation and cellular stress response pathways. EMBO J., 32: 1214.
4. Condamine, T et al., December 2015, Transcriptional regulation of myeloid-derived suppressor cells. J Leukoc Biol 98:913.
5. Condamine, T et al., June, 2014, ER stress regulates myeloid-derived suppressor cell fate through TRAIL-R-mediated apoptosis. J Clin Invest 124: 2626.
6. Condamine, T. et al., 2015 Regulation of tumor metastasis by myeloid-derived suppressor cells. Annu Rev Med 66, 97.
7. De Siqueira, J. et al., November 2015, Clinical and Preclinical Use of LOX-1-Specific Antibodies in Diagnostics and Therapeutics. J. Cardiovascular translational research 8, 458
8. Diaz-Montero, C M et al., January 2009, Increased circulating myeloid-derived suppressor cells correlate with clinical cancer stage, metastatic tumor burden, and doxorubicincyclophosphamide chemotherapy. Canc. Immunol Immunother 58: 49.
9. Feng P H, et al., November 2012, CD14(+)S100A9(+) monocytic myeloid-derived suppressor cells and their clinical relevance in non-small cell lung cancer. American J. Respiratory Crit. Care Med., 186: 1025.
10. Filipazzi, P., et al. 2007 Identification of a new subset of myeloid suppressor cells in peripheral blood of melanoma patients with modulation by a granulocyte-macrophage colony-stimulation factor-based antitumor vaccine. J Clin Oncol 25, 2546-2553
11. Finke, J et al., July 2011, MDSC as a mechanism of tumor escape from sunitinib mediated anti-angiogenic therapy. Int Immunopharmacol 11, 856.
12. Finkelstein, S. E. et al., October 2010, Changes in dendritic cell phenotype after a new high-dose weekly schedule of interleukin-2 therapy for kidney cancer and melanoma. J Immunother, 33: 817.
13. Fridlender Z G et al., 2012, Transcriptomic analysis comparing tumor-associated neutrophils with granulocytic myeloid-derived suppressor cells and normal neutrophils. PloS One, 7(2): e31524.
14. Gabrilovich, D. I. & Nagaraj, S. 2009 Myeloid-derived suppressor cells as regulators of the immune system. Nat Rev Immunol 9, 162-174
15. Gabrilovich, D. I., et al. 2012 Coordinated regulation of myeloid cells by tumours. Nat Rev Immunol 12, 253-268
16. Hirsch, H A et al., April 2010, A transcriptional signature and common gene networks link cancer with lipid metabolism and diverse human diseases. Cancer Cell, 17: 348
17. Holcik, M et al., April 2005, Translational control in stress and apoptosis. Nature Rev Mol Cell Biol, 6: 318.
18. Hong, D. et al., 2015, High-Density Lipoprotein Prevents Endoplasmic Reticulum Stress-Induced Downregulation of Liver LOX-1 Expression. PloS One, 10: e0124285
19. Hong, D. et al., August 2014 Ox-LDL induces endothelial cell apoptosis via the LOX-1-dependent endoplasmic reticulum stress pathway Atherosclerosis, 235: 310
20. Kimura, T et al., 2013 MUC1 vaccine for individuals with advanced adenoma of the colon: a cancer immunoprevention feasibility study. Cancer Prev Res, 6: 18
21. Kumar, V et al, March 2016, The nature of myeloid-derived suppressor cells in the tumor microenvironment. Trends Immunol., 37(3):208-220.
22. Lee, B R et al., December 2014, Elevated endoplasmic reticulum stress reinforced immunosuppression in the tumor microenvironment via myeloid-derived suppressor cells. Oncotarget, 5: 12331
23. Liu, C Y et al., January 2010, Population alterations of L-arginase- and inducible nitric oxide synthase-expressed CD11b+/CD14(−)/CD15+/CD33+ myeloid-derived suppressor cells and CD8+T lymphocytes in patients with advanced-stage non-small cell lung cancer. J Cancer Res Clin Oncol 136: 35.
24. Lu, J. et al, October 2011, Oxidative stress and lectin like ox-LDL-receptor LOX-1 in atherogenesis and tumorigenesis. Antioxid Redox Signal, 15, 2301
25. Mahadevan, N R et al., 2012, Cell-extrinsic effects of tumor ER stress imprint myeloid dendritic cells and impair CD8(+) T cell priming PloS One 7, e51845.
26. Mahadevan, N R et al., April 2011, Transmission of endoplasmic reticulum stress and proinflammation from tumor cells to myeloid cells. Proc Natl Acad Sci USA, 108: 6561
27. Mahadevan, N R et al., November 2011, Tumor stress inside out: cell-extrinsic effects of the unfolded protein response in tumor cells modulate the immunological landscape of the tumor microenvironment. J Immunol, 187: 4403
28. Mandruzzato, S et al., 2016 Toward harmonized phenotyping of human myeloid derived suppressor cells by flow cytometry: results from an interim study. Cancer Immunol Immunother, 65: 161.
29. Marvel, D. et al, September 2015, Myeloid-derived suppressor cells in the tumor microenvironment: expect the unexpected. J Clin Invest 125: 3356.
30. Mehta J L et al., LOX-1 Oct. 2011, a new target for therapy for cardiovascular diseases. Cardiovascular drugs and therapy/sponsored by the International Society of Cardiovascular Pharmacotherapy 25, 495
31. Mehta, J L et al., June 2007, Deletion of LOX-1 reduces atherogenesis in LDLR knockout mice fed high cholesterol diet. Circ Res, 100: 1634.
32. Messmer, M N et al., January, 2015, Tumor-induced myeloid dysfunction and its implications for cancer immunotherapy. Cancer Immunol Immunother 64, 1.
33. Meyer, C et al., March 2014, Frequencies of circulating MDSC correlate with clinical outcome of melanoma patients treated with ipilimumab. Canc Immunol Immunother 63: 247.
34. Montero, A. J., et al. 2012 Myeloid-derived suppressor cells in cancer patients: a clinical perspective J Immunother 35, 107-115
35. Pirillo, A. et al, 2013, LOX-1, OxLDL, and atherosclerosis. Mediators Inflamm 2013, 152: 786.
36. Poschke, I et al, September 2012, On the armament and appearances of human myeloid derived suppressor cells. Clinical Immunol 144: 250.
37. Ramachandran, I R et al., April 2013, Myeloid-derived suppressor cells regulate growth of multiple myeloma by inhibiting T cells in bone marrow. J Immunol, 190: 3815.
38. Ron, D et al, July 2007, Signal integration in the endoplasmic reticulum unfolded protein response. Nature RevMol Cell Biol., 8: 519.
39. Sawamura, A. et al, October 2012, LOX-1: a multiligand receptor at the crossroads of response to danger signals. Current Opin. Lipidol. 23: 439.
40. Sawamura, T. et al., March 1997, An endothelial receptor for oxidized low-density lipoprotein. Nature 386: 73.
41. Storey, J D et al, August 2003, Statistical significance for genomewide studies. Proc Natl Acad Sci USA, 100: 9440
42. Talmadge, J. E. & Gabrilovich, D. I. 2013 History of myeloid-derived suppressor cells. Nat Rev Cancer 13, 739-752
43. Tang, C H et al., June 2014, Inhibition of ER stress-associated IRE-1/XBP-1 pathway reduces leukemic cell survival. J Clin Invest 124: 2585
44. Tarhini, A A et al., 2014 Immune Monitoring of the Circulation and the Tumor Microenvironment in Patients with Regionally Advanced Melanoma Receiving Neoadjuvant Ipilimumab. PloS One, 9: e87705.
45. Taye, A. & El-Sheikh, A. A. 2013 Lectin-like oxidized low-density lipoprotein receptor 1 pathways. Eur J Clin Invest 43, 740-745
46. Thevenot, P T et al., 2014 The stress-response sensor chop regulates the function and accumulation of myeloid-derived suppressor cells in tumors. Immunity, 41: 389
47. Vetsika, E.-K. et al., 2014 A Circulating Subpopulation of Monocytic Myeloid-Derived Suppressor Cells as an Independent Prognostic/Predictive Factor in Untreated Non-Small Lung Cancer Patients. J. Immunol. Res., 65: 92-94.
48. Walter, P. et al, 2011, The unfolded protein response: from stress pathway to homeostatic regulation. Science, 334: 1081
49. Wang, X. et al, October 2011, LOX-1 and angiotensin receptors, and their interplay. Cardiovascular drugs and therapy/sponsored by the International Society of Cardiovascular Pharmacotherapy 25, 401.
50. Wang, Z et al., January 2014, Association of myeloid-derived suppressor cells and efficacy of cytokine-induced killer cell immunotherapy in metastatic renal cell carcinoma patients. J Immunother 37: 43.
51. Yoshimoto, R. et al., The discovery of LOX-1, its ligands and clinical significance. Cardiovascular drugs and therapy/sponsored by the International Society of Cardiovascular Pharmacotherapy 25, 379 (October, 2011).
52. Youn, J. I. et al. 2008 Subsets of myeloid-derived suppressor cells in tumor-bearing mice. J Immunol 181, 5791-5802
53. Youn, J. I., et al., 2012 Characterization of the nature of granulocytic myeloid-derived suppressor cells in tumor-bearing mice. J Leukoc Biol 91, 167-181
54. Zhang, K. et al, July 2008, From endoplasmic-reticulum stress to the inflammatory response. Nature, 454: 455
55. Zhang, S. 2007 A comprehensive evaluation of SAM, the SAM R-package and a simple modification to improve its performance. BMC bioinformatics, 8: 230
56. Nelson, A. January 2010, Antibody Fragments: Hope and Hype, MAbs, 2(1):77-83

1. A method comprising:
obtaining a biological sample from a subject;
contacting the sample with a ligand that specifically binds or forms a complex with a biomarker that forms a unique genomic signature in PMN-MDSC of a subject with a cancer that is distinguishable from neutrophils,
wherein said signature comprises the relative expression of two or more biomarkers of Table 1, FIG. 9A or FIG. 10B.

2. The method according to claim 1, further comprising contacting the sample with a reagent that identifies activators or regulators of ER stress response in said cells of the sample.

3. The method according to claim 2, wherein the activators are one or more of sXBP1, DDIT3 (CHOP), ATF4, ATF3, SEC61A ARGI or NOS-2.

4. The method according to claim 2, wherein the regulators or biomarkers are one or more of one or more of MYCN, CSF3, IL3, TGFβ1, TNF, LDL, RAF1, APP, IL6 PDGFBB, EPO, CD40LG, Nek, IL13, AGT, IL1β, ERBB2, MAP2K1, VEGFα, CSF1, FLI1, or Fin, CD15, CD66b or CD33.

5. A composition comprising a ligand that specifically binds or forms a complex with LOX-1 on the cell surface for use in the method of claim 1.

6. A pharmaceutical composition that reduces or inhibits ER stress in mammalian LOX-1+ neutrophils, LOX-1+ PMN or PMN-MDSC or reduces or inhibits LOX-1 expression on said cell populations in a pharmaceutically acceptable carrier or excipient.

7. The composition according to claim 6, comprising an antagonist or inhibitor of the expression, activity or activation of one or more of sXBP1, DDIT3 (CHOP), ATF4, ATF3, SEC61A ARGI or NOS-2.

8. The composition according to claim 6, wherein said composition comprises an antagonist or inhibitor of LOX-1.

9. The composition according to claim 6, wherein said composition comprises an antagonist or inhibitor of the expression, activity or activation of one or more of MYCN, CSF3, IL3, TGFβ1, TNF, LDL, RAF1, APP, IL6 PDGFBB, EPO, CD40LG, Nek, IL13, AGT, IL1β, ERBB2, MAP2K1, VEGFα, CSF1, FLI1, Fin, CD15, CD66b or CD33.

10. The composition according to claim 6, wherein the composition comprises an antibody or functional antigen-binding fragment thereof.

11. A method for reducing or inhibiting LOX-1+ PMN-MDSC accumulation in a cancer patient comprising administering a composition of claim 6.

12. A method of treating a cancer comprising:
(a) administering an effective amount of a composition that reduces or inhibits ER stress response in mammalian LOX-1⁺ neutrophils, LOX-1⁺ PMN or PMN-MDSC or reduces or inhibits LOX-1 expression on LOX-1⁺ neutrophils, LOX-1⁺ PMN or PMN-MDSC; or
(b) i. obtaining a biological sample from a subject;
ii. contacting the sample with a ligand that specifically binds or forms a complex with a biomarker that forms a unique genomic signature in PMN-MDSC that is distinguishable from neutrophils;
iii. detecting whether PMN-MDSC are present in the sample; and
iv. when the presence of LOX-1+ is detected at a level that indicates PMN-MDSC are present, either administering an effective amount of a composition that reduces or inhibits ER stress response in mammalian neutrophils or reduces or inhibits LOX-1 expression on neutrophil populations; or
(c) i. obtaining a biological sample from the subject;
ii. detecting whether soluble LOX-1 is present in the sample by contacting the sample with an antibody or functional antibody fragment that specifically binds or forms a complex with LOX-1 on the cell surface;
iii. detecting and distinguishing the complexes of antibody-bound LOX-1-cells from other cells not bound to the antibody in the sample, and
iv. determining the size of a tumor in the subject by correlation with the number of LOX-1+ PMN or PMN-MDSC detected.

13. The method according to claim 12, wherein the composition that reduces or inhibits the ER stress response comprises an antibody or functional antigen-binding fragment that binds to LOX-1 or comprises an antibody or functional antigen-binding fragment that binds to or inhibits the expression, activity or activation of at least one of sXBP1, DDIT3 (CHOP), ATF4, ATF3, SEC61A ARGI, MYCN, CSF3, IL3, TGFβ1, TNF, LDL, RAF1, APP, IL6 PDGFBB, EPO, CD40LG, Nek, IL13, AGT, IL1β, ERBB2, MAP2K1, VEGFα, CSF1, FLI1, Fin, CD15, CD66b or CD33.

14. The method according to claim 12, wherein the detecting step of (b) comprises contacting the sample with an antibody or functional antigen-binding fragment that binds to LOX-1 or comprises an antibody or functional antigen-binding fragment that binds or inhibits the expression, activity or activation of at least one of sXBP1, DDIT3 (CHOP), ATF4, ATF3, SEC61A ARGI, MYCN, CSF3, IL3, TGFβ1, TNF, LDL, RAF1, APP, IL6 PDGFBB, EPO, CD40LG, Nek, IL13, AGT, IL1β, ERBB2, MAP2K1, VEGFα, CSF1, FLI1, or Fin or CD15, CD66b or CD33.

15. The method according to claim 12, further comprising in (c) detecting the presence of CD15 in said sample.

16. The method according to claim 12, further comprising in (c) contacting the sample with a ligand that specifically binds or forms a complex with a biomarker that forms a unique genomic signature in PMN-MDSC, wherein said signature comprises the relative expression of two or more biomarkers of Table 1, FIG. 9A or FIG. 10B.


SEQUENCE LISTING (16 sequences)

SEQ ID NO: 1 - DNA, 18 bp, Artificial Sequence
  5′ primer sequence for sXBP-1
ctgagtccgcagcaggtg

SEQ ID NO: 2 - DNA, 21 bp, Artificial Sequence
  3′ primer sce for sXBP-1
agttgtccagaatgcccaaca

SEQ ID NO: 3 - DNA, 25 bp, Artificial Sequence
  5′ primer sequence for DDIT3
gcacctcccagagccctcactctcc

SEQ ID NO: 4 - DNA, 25 bp, Artificial Sequence
  3′ primer for DDIT3
gtctactccaagccttccccctgcg

SEQ ID NO: 5 - DNA, 21 bp, Artificial Sequence
  5′ primer for ATF4
ttcctgagcagcgaggtgttg

SEQ ID NO: 6 - DNA, 21 bp, Artificial Sequence
  3′ primer sequence for ATF4
tccaatctgtcccggagaagg

SEQ ID NO: 7 - DNA, 20 bp, Artificial Sequence
  5′ primer sequence for ATF3
tgcctcggaagtgagtgctt

SEQ ID NO: 8 - DNA, 22 bp, Artificial Sequence
  3′ primer sequence for ATF3
gcaaaatcctcaaacaccagtg

SEQ ID NO: 9 - DNA, 20 bp, Artificial Sequence
  5′ primer sequence for SEC61A
ggatgtatggggacccttct

SEQ ID NO: 10 - DNA, 20 bp, Artificial Sequence
  3′ primer sequence for SEC61A
ctcggccagtgttgacagta

SEQ ID NO: 11 - DNA, 20 bp, Artificial Sequence
  5′ primer sequence for ARGI
cttgtttcggacttgctcgg

SEQ ID NO: 12 - DNA, 21 bp, Artificial Sequence
  3′ primer sequence for ARGI
cactctatgtatgggggctta

SEQ ID NO: 13 - DNA, 19 bp, Artificial Sequence
  5′ primer sequence for NOS-2
cagcgggatgactttccaa

SEQ ID NO: 14 - DNA, 20 bp, Artificial Sequence
  3′ primer sequence for NOS-2
aggcaagatttggacctgca

SEQ ID NO: 15 - DNA, 22 bp, Artificial Sequence
  5′ primer sequence for GADPH
ggagtcaacggatttggtcgta

SEQ ID NO: 16 - DNA, 26 bp, Artificial Sequence
  3′ primer sequence for GADPH
ggcaacaatatccactttaccagagt